SEQUENCE LISTING (26 sequences)

SEQ ID NO: 1 - DNA, 46 bp, Artificial Sequence
  IntPhiC31attB(TT)
gtgcgggtgccagggcgtgcccttgggctccccgggcgcgtactcc

SEQ ID NO: 2 - DNA, 46 bp, Artificial Sequence
  IntPhiC31attP(TT)
agtgccccaactggggtaacctttgagttctctcagttgggggcgt

SEQ ID NO: 3 - DNA, 46 bp, Artificial Sequence
  IntPhiC31attB(AG)
gtgcgggtgccagggcgtgcccaggggctccccgggcgcgtactcc

SEQ ID NO: 4 - DNA, 46 bp, Artificial Sequence
  IntPhiC31attP(AG)
agtgccccaactggggtaacctaggagttctctcagttgggggcgt

SEQ ID NO: 5 - DNA, 49 bp, Artificial Sequence
  IntBxb attB(GA)
cggccggcttgtcgacgacggcggactccgtcgtcaggatcatccgggc

SEQ ID NO: 6 - DNA, 55 bp, Artificial Sequence
  IntBxb attP(GA)
gtcgtggtttgtctggtcaaccaccgcggactcagtggtgtacggtacaaaccca

SEQ ID NO: 7 - DNA, 73 bp, Artificial Sequence
  LambdaPR(T41C)a
tatcaccgcaagggataaatatctaacaccgcgcgtgttgactattttacctctggcggtgataatggttgca

SEQ ID NO: 8 - DNA, 61 bp, Artificial Sequence
  LambdaPL
aaccatctgcggtgataaattatctctggcggtgttgacataaataccactggcggtgata

SEQ ID NO: 9 - DNA, 141 bp, Artificial Sequence
  PkatG
taatcaaaaaagcttaattaagatcaatttgatctacatctctttaaccaacaatatgtaagatctcaactatcgcatccgtggattaattcaattataacttctctctaacgctgtgtatcgtaacggtaacactgtaga

SEQ ID NO: 10 - DNA, 27 bp, Artificial Sequence
  oAJ32
ctcgattctattaacaagggtatcacc

SEQ ID NO: 11 - DNA, 19 bp, Artificial Sequence
  oAJ91
gtgcgggtgccagggcgtg

SEQ ID NO: 12 - DNA, 19 bp, Artificial Sequence
  oAJ297
ggggagcccaaaggttacc

SEQ ID NO: 13 - DNA, 3835 bp, Artificial Sequence
  f_bAJ78
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaagtagggaactgccaggcatgggagaccagaaaagaaaaaacacccgttagggtgtttttagttagaacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatgccgtcaattgtctgattcgttaccaaaaaggcttgacaattaatcatcggctcgtataatgtgtggaaagtgccccaactggggtaacctttgagttctctcagttgggggcgtttaataaactatggaagtatgtacagtcttgcaatgttgagtgaacaaacttccataataaaattgcgagtttttatttcgtttatttcaattaaggtaactaaaaaactcctttacgtcgtggtttgtctggtcaaccaccgcggtctcagtggtgtacggtacaaacccaattattgaagcggctaacgccgctttttttgtttctggtctcccctcggtaccaaattccagaaaagacacccgaaagggtgttttttcgttttggtccgctatagcgctcgaagggatcgctaagcgctagagcttaatactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtctcggccggcttgtcgacgacggcggtctccgtcgtcaggatcatccgggcatgccaacacaattaacatctcaatcaaggtaaatgctttttgctttttttgccaatcatcagataactatggcggcacgtgcattaaccacggttgtatcccgtctaaagtactcgtgtgcgggtgccagggcgtgcccttgggctccccgggcgcgtactcccactgctctttaacaatttatcagatccaataggaggaacaatatgaaaggatccaaaggtgaagaattattcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaaagtatacatcatggcagacaaacaaaaggatggaatcaaagttaacttcaaaattagacacaacattgaagatggaagcgttcaactagcagaccattatcaacgaaatactccaattggcgatggccctgtccttttaccagacaaccattacctgtccacacaatctgccctttcgaaagatcccaacgaaaagagagaccacatggtccttcttgagtttgtaacagctgctgggattacacatggtatggatgaattgtacaaataacgcaaaaaaccccgcccctgacagggcggggttttttcgcgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 14 - DNA, 9278 bp, Artificial Sequence
  oriC region of bAJ174
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtcgccgctaccatggtgatggtgatggtgcatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttctgatcggcacgtaagaggttccaactttcaccataatgaaataagatcactaccgggcgtattttttgagttgtcgagattttcaggagctaaggaagctaaaaacttttcggatcaagctatgaaaaaagaagaagaaacgagaaactaaacgtccttgcggaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaggcgcgccatttaaatgaagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtaacaattgggatccactgctctttaacaatttatcagatccaataggaggaacaatatgaaaggatccaaaggtgaagaattattcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaaagtatacatcatggcagacaaacaaaaggatggaatcaaagttaacttcaaaattagacacaacattgaagatggaagcgttcaactagcagaccattatcaacgaaatactccaattggcgatggccctgtccttttaccagacaaccattacctgtccacacaatctgccctttcgaaagatcccaacgaaaagagagaccacatggtccttcttgagtttgtaacagctgctgggattacacatggtatggatgaattgtacaaataacgcaaaaaaccccgcccctgacagggcggggttttttcgctttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 15 - DNA, 4083 bp, Artificial Sequence
  pAJ27
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgcatgtactaccggatatagttcctcctttcagcaaaaaacccctcaagacccgtttagaggccccaaggggttatgctagttattgctcagcggtggcagcagcctaggttaattaagctgcgctagtagacgagtccatgtgctggcgttcaaatttcgcagcagcggtttctttaccagactcgagtcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatatgtatatctccttcttatacattcaccaccctgaattgactctcttccgggcgctatcatgccataccgcgaaaggtttgcgccattcgatggtgtcgccgtcaattgtctgattcgttaccaatatcaccgcaagggataaatatctaacaccgcgcgtgttgactattttacctctggcggtgataatggttgcatgtactcagatccaataggaggaacaatatggacacgtacgcgggtgcttacgaccgtcagtcgcgcgagcgcgaaaattcgagcgcagcaagcccagcgacacagcgtagcgccgatggtagtgagagaatataaaaagccagattattaatccggcttttttattattttgccaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaa

SEQ ID NO: 16 - DNA, 5841 bp, Artificial Sequence
  pAJ35
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgcatgtactaccggatatagttcctcctttcagcaaaaaacccctcaagacccgtttagaggccccaaggggttatgctagttattgctcagcggtggcagcagcctaggttaattaagctgcgctagtagacgagtccatgtgctggcgttcaaatttcgcagcagcggtttctttaccagactcgagtcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatatgtatatctccttcttatacattcaccaccctgaattgactctcttccgggcgctatcatgccataccgcgaaaggtttgcgccattcgatggtgtcgccgtcaattgtctgattcgttaccaatatcaccgcaagggataaatatctaacaccgcgcgtgttgactattttacctctggcggtgataatggttgcatgtactcagatccaatagaaggaacaatatggacacgtacgcgggtgcttacgaccgtcagtcgcgcgagcgcgaaaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtagaactcagaagtgaaacgccgtagcgccgatggtagtgagagaatataaaaagccagattattaatccggcttttttattattttgccaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaa

SEQ ID NO: 17 - DNA, 9174 bp, Artificial Sequence
  pAJ144
gggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcgacggatcgatccggggaattaattccggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccacctgcatcgcaagctagcttgctagagggtcagctttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcataaagctgactctagctagaggatcttcgaatgcatcgcgcgcaccgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgaattcacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccatggtgacgtcaccggttctagatacctaggtgagctctggtaccgcggccgatcttcgtccacgccgagccctatgcagtctccgattatgttaaccagtatgtcggtacgcactctattcgcctgcccaagggcgggcccccggcaggcaggctgcaccacagaatcttcggatgcctcgacctgtgtcgaatcagctacggcggtagcgtgagggtaatctcgcctggattagagacctgttatcatctgcaaataatactcaaaggccattgcctgtggcgtggccatggccaggagcactattttgcgccgggcgaactattgctgctcaatccggatgaccaagccgacctgacctattcagaagattgcgagaaatttatcgttaaattgccctcagtggtccttgatcgggcatgcagtgacaacaattggcacaagccgagggagggtatccgtttcgccgcgcgacacaatctccagcaactcgatggctttatcaatctactcgggttagtttgtgacgaagcggaacatacaaagtcgatgcctcgggtccaagagcactatgcggggatcatcgcttccaagctgctcgaaatgctgggcagcaatgtcagccgtgaaattttcagcaaaggtaacccgtctttcgagcgagtcgttcaattcattgaggagaatctcaaacggaatatcagccttgagcggttagcggagctggcgatgatgagtccacgctcgctctacaatttgttcgagaagcatgccggcaccacgccgaagaactacatccgcaaccgcaagctcgaaagcatccgcgcctgcttgaacgatcccagtgccaatgtgcgtagtataactgagatagccctagactacggcttcttacatttgggacgcttcgctgaaaactataggagcgcgttcggcgagttgccttccgacaccctgcgtcaatgcaaaaaggaagtggcttgattacgaacgtagccgaagaagggatgggttggcatcgcccggctttcttagacactctccaagctctgaaatagcgttttacaaactccactggctatagtgccggcgtttgcccgcgctctccgtggccgcgtcccaatgcaggtcgggttcctcgacccgaagtgcccttgggcatctccacttcagcgtctgaccttgctggccagttcgccgccgatgccgaagtggcccttggccatctccgtgataatcactcgcacgctggtcagcggcgcatccagggagcgcgagatggcctcgctgacttcccgaatgagggtttccttctgctcgtcgctgcggccttcaaggatgtggatctgctggccgaaagggggatgtgctgcaaggcgattaagttgggtaacgccagggttttcccagtcacgacgttgtaaaacgacggccagtgaattaattcttgaagacgaaagggcctcgtgatacgcctatttttataggttaatgtcatgataataatggtttcttagagcttacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcggctgcagcgatccgtcgatctatctcatctgcgcaaggcagaacgtgaagacggccgccctggacctcgcccgcgagcgccagcgcacgaggccggcgcgcggacccgccgcggcccacgagcggacgccgcagcaggagcgccagaaggccgccagagaggccgagcgcggcgtgaggcttggacgctagggcagggcatgaaaaagcccgtagcgggcgctacggggctctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggactcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccagtcagaaatgagcgccagtcgtcgtcggctctcggcaccgaagtgctatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtctagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcagcttcgttcggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgccaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgatagggacggatcgctgcagcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgggggatcaggaccgctgccggagcgcaacccactcactacagcagagccatgta

SEQ ID NO: 18 - DNA, 4343 bp, Artificial Sequence
  pAJ157
tttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgctcccggccgccatggcggccgcgggaattcgatttgggccttttgacaataatcatcggctcgtataatgtgtggaattgtgagcggataacaattgggatccgcctttaacaatttatcagatccaataggaggagatctatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagtaggtcgacaagcttgcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtattgaggggttttttgctggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtt

SEQ ID NO: 19 - DNA, 5560 bp, Artificial Sequence
  pAJ194
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgctcccggccgccatggcggccgcgggaattcgatttgggccttttgacaataatcatcggctcgtataatgtgtggaattgtgagcggataacaattgggatccgctctttaacaatttatcagatccaataggaggagaatctatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccaccaccacctgttccagtaggtcgacaagcttgcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaa

SEQ ID NO: 20 - DNA, 1845 bp, Artificial Sequence
  codon optimised integrase from phage PhiC31
atgcaccatcaccatcaccatggtagcggcgacacgtacgcgggtgcttacgatcgccaatctcgtgaacgtgaaaacagctcagccgcgtcccccgcaacccaacgctccgcgaatgaggataaagccgcagatctgcaacgtgaggtggaacgtgatggtggtcgctttcgttttgtgggtcacttttcagaggctccagggaccagcgcttttggtacagccgaacgtcctgaatttgagcgtattttaaatgagtgtcgtgcgggccgcttaaatatgattatcgtgtacgatgtttctcgcttttctcgtctcaaagtgatggatgcaatccccatcgtaagcgagttactggcgttaggtgtcaccatcgtctcaacccaagagggtgtgtttcgccaaggcaatgtcatggacctgattcacctgatcatgcgtctggatgcctcacacaaggagagcagtttaaagagcgccaaaatcctggatactaagaatttacaacgtgagctgggtggatatgtgggcggtaaagcgccttacggcttcgagcttgttagcgaaaccaaagaaattacccgtaatggtcgcatggttaacgtggtgattaataaactggcccattcaacaaccccgctgacaggcccgtttgaatttgaaccagatgtcattcgttggtggtggcgcgaaattaaaactcataagcatctgccgtttaaaccaggttcccaggcggcgatccatcctggttcaattaccggcttgtgcaaacgtatggatgcggatgcggttccaacgcgtggtgaaaccatcggcaagaaaactgcgagctcggcgtgggaccccgccacagtgatgcgcattttacgcgatccccgcatcgccggatttgctgcggaagtcatttataagaagaaaccagatgggaccccaacgaccaaaatcgaagggtatcgtatccaacgtgatcctattaccctgcgtcctgttgaactggactgtggccccattattgaacctgcggaatggtacgaactgcaagcctggctggatggtcgcggtcgtggtaaaggtctgagccgtggtcaggcgatcttaagcgcgatggataaactttattgtgaatgcggtgcggttatgacatccaaacgtggcgaggagtcaattaaagattcatatcgttgtcgccgtcgcaaagtcgttgatccctcggcgccgggccaacatgagggaacttgtaatgtgtctatggccgcgctggataaatttgtagccgagcgtatctttaataaaattcgtcatgcagagggtgatgaggaaaccctggcattgttatgggaagcggcgcgccgttttggtaaacttaccgaagccccggagaaatcaggagagcgcgccaatctggtggccgaacgtgcggatgcgctgaacgcccttgaagagctgtatgaggatcgtgcggccggggcatatgatggtccggtcggtcgtaaacattttcgcaaacagcaagcggcattaaccttacgtcaacagggcgccgaggaacgtctggcagagctcgaggcggccgaggcacccaaattgccgctggatcagtggtttccggaagatgcggatgcggacccaacaggtccgaaaagctggtggggccgtgcaagtgtggatgataaacgtgtatttgtgggcctgtttgttgataaaattgtggtgaccaaatcaacgaccggacgcggccaaggcaccccgattgagaaacgtgcgagtattacttgggctaaacccccaacggatgatgatgaggatgatgctcaagacggcacggaagacgtagcggcgtaa

SEQ ID NO: 21 - DNA, 1503 bp, Artificial Sequence
  codon optimised integrase from phage Bxb1
atgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataa

SEQ ID NO: 22 - DNA, 714 bp, Artificial Sequence
  codon optimised repressor cI857
atgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaa

SEQ ID NO: 23 - DNA, 9226 bp, Artificial Sequence
  pAJ144
gggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcgacggatcgatccggggaattaattccggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccacctgcatcgcaagctagcttgctagagggtcagctttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcataaagctgactctagctagaggatcttcgaatgcatcgcgcgcaccgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccatggtgacgtcaccggttctagatacctaggtgagctctggtaccgcggccgatcttcgtccacgccgagccctatgcagtctccgattatgttaaccagtatgtcggtacgcactctattcgcctgcccaagggcgggcccccggcaggcaggctgcaccacagaatcttcggatgcctcgacctgtgtcgaatcagctacggcggtagcgtgagggtaatctcgcctggattagagacctgttatcatctgcaaataatactcaaaggccattgcctgtggcgtggccatggccaggagcactattttgcgccgggcgaactattgctgctcaatccggatgaccaagccgacctgacctattcagaagattgcgagaaatttatcgttaaattgccctcagtggtccttgatcgggcatgcagtgacaacaattggcacaagccgagggagggtatccgtttcgccgcgcgacacaatctccagcaactcgatggctttatcaatctactcgggttagtttgtgacgaagcggaacatacaaagtcgatgcctcgggtccaagagcactatgcggggatcatcgcttccaagctgctcgaaatgctgggcagcaatgtcagccgtgaaattttcagcaaaggtaacccgtctttcgagcgagtcgttcaattcattgaggagaatctcaaacggaatatcagccttgagcggttagcggagctggcgatgatgagtccacgctcgctctacaatttgttcgagaagcatgccggcaccacgccgaagaactacatccgcaaccgcaagctcgaaagcatccgcgcctgcttgaacgatcccagtgccaatgtgcgtagtataactgagatagccctagactacggcttcttacatttgggacgcttcgctgaaaactataggagcgcgttcggcgagttgccttccgacaccctgcgtcaatgcaaaaaggaagtggcttgattacgaacgtagccgaagaagggatgggttggcatcgcccggctttcttagacactctccaagctctgaaatagcgttttacaaactccactggctatagtgccggcgtttgcccgcgctctccgtggccgcgtcccaatgcaggtcgggttcctcgacccgaagtgcccttgggcatctccacttcagcgtctgaccttgctggccagttcgccgccgatgccgaagtggcccttggccatctccgtgataatcactcgcacgctggtcagcggcgcatccagggagcgcgagatggcctcgctgacttcccgaatgagggtttccttctgctcgtcgctgcggccttcaaggatgtggatctgctggccgaaagggggatgtgctgcaaggcgattaagttgggtaacgccagggttttcccagtcacgacgttgtaaaacgacggccagtgaattaattcttgaagacgaaagggcctcgtgatacgcctatttttataggttaatgtcatgataataatggtttcttagagcttacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcggctgcagcgatccgtcgatctatctcatctgcgcaaggcagaacgtgaagacggccgccctggacctcgcccgcgagcgccagcgcacgaggccggcgcgcggacccgccgcggcccacgagcggacgccgcagcaggagcgccagaaggccgccagagaggccgagcgcggcgtgaggcttggacgctagggcagggcatgaaaaagcccgtagcgggcgctacggggctctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggactcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccagtcagaaatgagcgccagtcgtcgtcggctctcggcaccgaagtgctatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtctagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcagcttcgttcggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgccaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgatagggacggatcgctgcagcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgggggatcaggaccgctgccggagcgcaacccactcactacagcagagccatgta

SEQ ID NO: 24 - DNA, 51 bp, Artificial Sequence
  Figure 2C - Predicted sequence
gaaagtgccccaactggggtaacctttgggctccccgggcgcgtactccca

SEQ ID NO: 25 - DNA, 51 bp, Artificial Sequence
  Figure 2C - Predicted sequence - complementary strand
tgggagtacgcgcccggggagcccaaaggttaccccagttggggcactttc

SEQ ID NO: 26 - DNA, 51 bp, Artificial Sequence; identical to SEQ ID NO: 24
  Figure 2C - Actual sequence

(19) United States
(12) Patent Application Publication
(10) Pub. No.: US 2024/0093210 A1
(43) Pub. Date: Mar. 21, 2024

(54) BIOSYNTHESIS METHOD

(71) Applicant: UNIVERSITY OF TARTU, Tartub (EE)

(72) Inventors: Arvi JÕERS, Tartu (EE); Marje KASARI, Õssu küla (EE); Villu KASARI, Õssu küla (EE); Mirjam KÄRMAS, Viljandi (EE)

(21) Appl. No.: 18/039,929

(22) PCT Filed: Dec. 3, 2021
(86) PCT No.: PCT/EP2021/084194

§ 371 Date: Jun. 1, 2023

(30) Foreign Application Priority Data
Dec. 4, 2020 (GB) 2019175.5

(51) Int. Cl. C12N 15/70

(52) U.S. Cl. CPC C12N 15/70

(57) ABSTRACT

The present invention relates generally to methods of producing products by bacterial cells. More particularly, the present invention relates to methods of producing products by bacterial cells, the methods comprising a step of irreversibly inactivating an origin of replication in the bacterial cell. The present invention also relates to modified bacterial cells, polynucleotide vectors, and uses thereof for producing products.

The present invention relates generally to methods of producing products by bacterial cells and in particular to the field of industrial biotechnology or microbiology where living cells are used to produce useful chemicals and products, e.g. biological products. More particularly, the present invention relates to a method of producing a product by a bacterial cell, the method comprising a step of irreversibly inactivating an origin of replication in the bacterial cell. The invention also relates to bacterial cells comprising an origin of replication, wherein the bacterial cells have been modified such that the origin of replication can be irreversibly inactivated. The invention also relates to modified bacterial cells, which lack a functional origin of replication sequence, as well as various vectors and uses of the modified bacterial cells.

Industrial biotechnology is using living cells, mostly microbes, to produce useful chemicals and materials. It has its roots in traditional biotechnology as people have been using microbes to produce sour milk, beer and pickled vegetables for thousands of years. More recently the selection of possible products has expanded greatly thanks to advancements in genetic engineering and synthetic biology.

There are reports of hundreds of different chemicals produced by microbes in the scientific literature, but only a fraction of them have reached commercial production. The main problem is that many candidate strains do not produce enough product; i.e. titer, yield and/or productivity are too low to enable profitable commercial production (Van Dien, 2013). The fundamental reason for low production is that there is a trade-off between growth of the microbe and production of the product (Venayak et al., 2015). In general terms, microbes can use the carbon and energy sources available to them either to grow by producing biomass, or to synthesize the product of interest. If more of the resources are allocated for growth, production will suffer, and vice versa. This growth versus production trade-off severely restricts the development of production strains and limits the list of products that can be produced commercially.

Two-stage bioprocess has been suggested as a way to bypass this trade-off (Burg et al., 2016). In a two-stage bioprocess, the bioprocess is divided into two stages—at first the cells grow at maximum rate without (significant) production and, when enough biomass has accumulated, growth is turned off and product production is induced. There are several well-described systems for inducing the expression of genes necessary for product synthesis and inducing product production (Marschall, Sagmeister and Herwig, 2017), but turning off growth is much harder to achieve. Solutions for turning off growth in two-stage bioprocess described so far rely on the re-arrangement of metabolic pathways (Soma et al., 2014)) (Brockman and Prather, 2015)) At the time of switching from growth to production, a metabolic pathway necessary for growth is turned off, and accumulating metabolic intermediates are channeled towards the product of interest. These solutions, however, are product-specific, and a new pathway for cessation of growth must be designed and built for every new product. Hence, a universal method for efficiently switching bacteria from the growth stage to the production stage is lacking.

The inventors have developed a novel method of two stage bioprocess and hence a novel method of product production by bacteria. Advantageously the present solution is universal, in the sense that it can be applied to all bacteria and is independent of any metabolic pathway of interest (e.g. it is independent of any metabolic pathway involved in production of the desired product). It is based on irreversible inactivation of the origin of replication of the bacterial chromosome. Surprisingly, it has been found that this irreversibly stops cell division and growth while leaving the cells metabolically active.

Possible applications of this invention are numerous. It can be used in microbial bioproduction to increase titer, yield and productivity of products, for example proteins and small molecule products. It can also be used to generate microbial consortiums or microbial co-cultures with a fixed strain ratio as non-dividing cells do not outgrow each other. Such consortiums can be used for production of products when the pathway is split between two (or more) strains or species. In this case the first strain produces an intermediate molecule that is then converted into the final product by the second (or further) strain (Wang et al., 2018)(Yuan et al., 2020). Using the invention the growth phase of these strains can be carried out separately and the consortium can be created by mixing the strains together in a pre-defined ratio after an origin of replication of each strain has been irreversibly inactivated, e.g. after cell growth and division has been completed. Alternatively, the strains can be mixed before an origin of replication of each strain has been irreversibly inactivated, e.g. before cell growth and division begins or has completed.

Separation of growth and production phase will also enable to conduct these at different times or facilities. It is possible to grow the cells at one facility and then store them for later use either at the same spot or transported elsewhere.

The present invention can be used also outside of industrial microbiology. In the field of living therapeutics, live microbial cells are administered to patients as therapeutic agents to produce necessary molecules or break down harmful ones. These live therapeutic agents should be metabolically active to fulfil their function, but their runaway multiplication should be avoided. The present invention possesses this dual functionality.

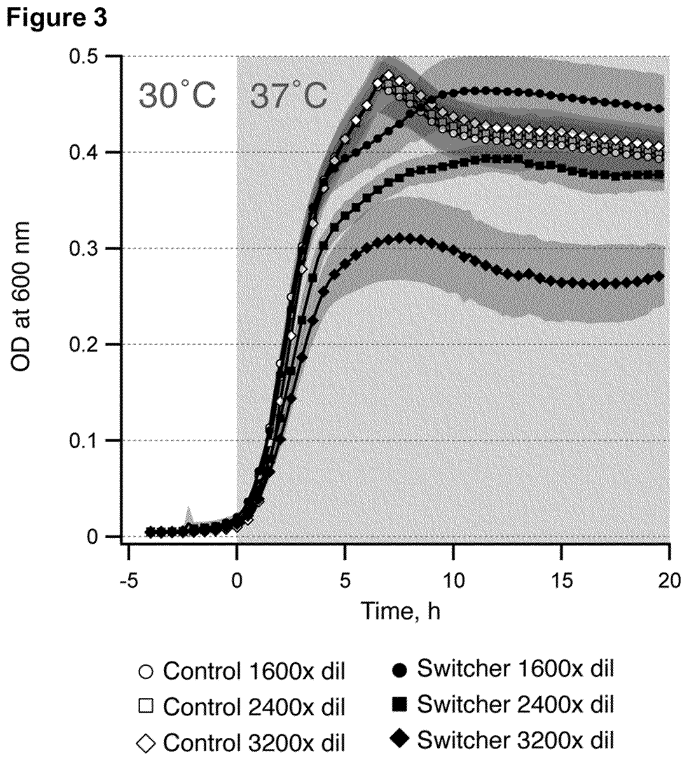

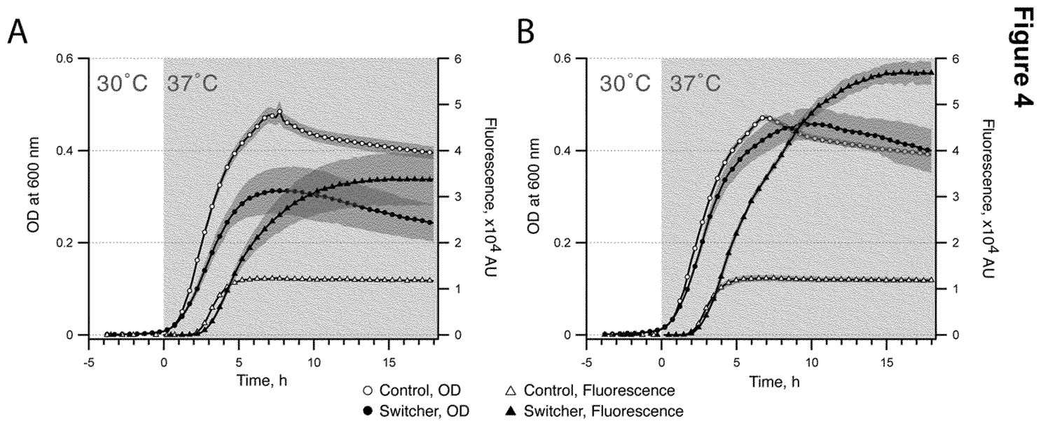

The method of the present invention facilitates and involves permanent removal or inactivation of the origin of replication from the genome. Once the origin of replication is removed or inactivated, the genome can no longer initiate replication. Since the efficiency of switching (e.g. origin of replication excision) is very high, the researcher may precisely select the final cell density at which the cell culture should reach before product production is induced (see e.g. FIG. 3).

Another advantage of the permanent nature of the origin of replication removal or inactivation used in the present invention is that it is not necessary to maintain the condition(s) required for switching of the bacteria from growth phase to production phase. Hence, once the origin of replication has been removed (or deleted or otherwise irreversibly inactivated), the cells can be returned to pre-induction conditions (or indeed any other conditions) without them resuming growth again. This gives more freedom to the operator and does not limit possible production conditions.

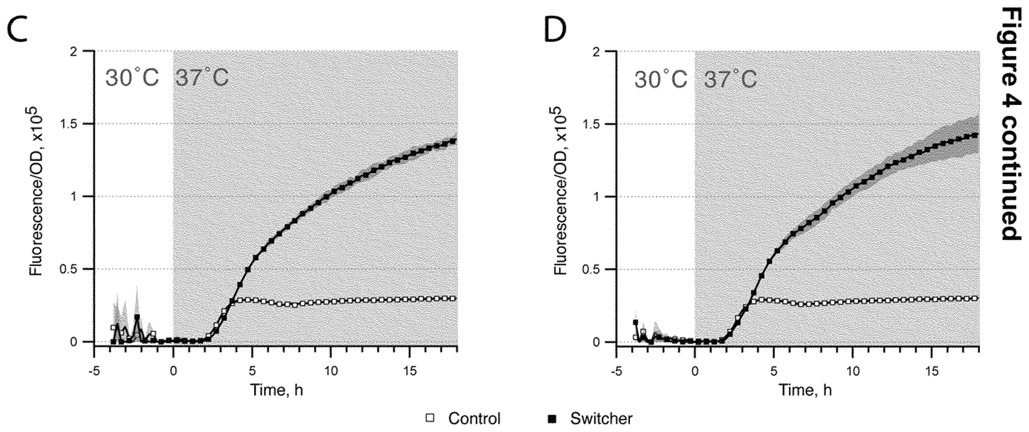

An unexpected advantage of the present technology is that switched cells keep producing product even at high culture density. In FIG. 4B it is demonstrated that in an experimental setup where both control and switcher strains (after switching) reach the same final cell density at the same time (approximately 10 hours), protein production continues far longer in switched cells than in control cells. In control cells, the red fluorescent protein (RFP) production is stopped at the 8 h time point and no more RFP accumulates thereafter. In switcher cells (after switching) however, RFP signal keeps increasing until 14 h, leading to up to 400% increase in RFP levels compared to control. This happens despite both cultures reaching the same maximum cell density at the same time.

The present invention has a wide scope of possible applications. For example, it can be used to generate a non-dividing preparation of cells for therapeutic use in the human body. In such applications, the permanent removal or inactivation of the origin of replication is vital, since it allows the maintenance of the inactivated state of the origin of replication when the cell preparation is inside the human body. This is important to prevent unwanted growth of the cells in the body whilst still allowing product (therapeutic product) production and hence the therapeutic effect.

The present invention can also be applied to the field of biocontainment, preventing runaway multiplication of cells that can accidentally escape from e.g. a bioreactor (or other confined environment) to a natural (or non-confined) environment (Lee et al., 2018). Since switched cells of the present invention cannot multiply they would pose a reduced threat if released from a confined (e.g. bioreactor) environment. In contrast for example, temporarily blocked cells could resume growth once they escaped induced conditions.

In one aspect, the invention provides a method of producing a product by a bacterial cell, the method comprising a step of irreversibly (or permanently) inactivating an origin of replication in the bacterial cell.

The term “origin of replication” is a term of the art and is understood by the skilled person. It refers generally to any sequence, e.g. nucleotide sequence, at which DNA replication can be initiated, for example by the binding of appropriate proteins and enzymes that are part of the replication machinery or necessary for the replication process. The origin of replication in a particular bacterial strain may be referred to in the art by a specific name. For example, the origin of replication as found in the chromosome of Escherichia coli is referred to as oriC and irreversible inactivation of this origin of replication is preferred in some embodiments. The origin of replication may be present on a chromosome, or alternatively on extra-chromosomal genetic material, for example an episome, plasmid or polynucleotide vector. Typically the origin of replication is located on a chromosome.

The terms “episome”, “plasmid”, and “polynucleotide vector” refer to an extra-chromosomal element often in the form of circular double-stranded DNA fragments. Such elements can be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence of interest or relevance, e.g. for a selected gene product, along with appropriate 3′ untranslated sequence into a cell.

The term “irreversibly inactivating” or “permanently inactivating” an origin of replication or similar as used herein means that the origin of replication is made irreversibly (or permanently) incapable of acting as a site of initiation of DNA replication or otherwise is made functionally inactive as an origin of replication.

Similarly, the term “irreversibly inactivatable” or “permanently inactivatable” origin of replication as used herein means an origin of replication which is active or functional but which can be (or is capable of being) inactivated in a way which is irreversible (or permanent).

Similarly, the term “irreversibly removable” or “permanently removable” origin of replication as used herein means an origin of replication which is active or functional but which can be (or is capable of being) removed in a way which is irreversible (or permanent).

In embodiments, irreversibly (or permanently) inactivating an origin of replication may be achieved through a permanent change, preferably a permanent genetic change (or permanent nucleotide change) to the origin of replication. An irreversibly (or permanently) inactivated origin of replication is an origin of replication that can no longer function as an origin of replication, for example can no longer be used as a site where DNA replication is initiated.

In embodiments, irreversibly (or permanently) inactivating an origin of replication in the bacterial cell comprises or consists of genetic modification (or nucleotide modification) of the origin of replication.

In embodiments, genetic modification (or nucleotide modification) comprises or consists of mutation of the origin of replication such that it can no longer function as an origin of replication.

In embodiments, mutation of the origin of replication comprises mutation of the origin of replication by insertion, removal, or substitution of nucleotides from the origin of replication. Exemplary mutations or modifications or other alterations are those such that proteins, enzymes or other molecular entities which are required to interact with the origin of replication in order for replication to be initiated or carried out can no longer interact with the origin of replication, or can no longer interact with the origin of replication in such a way that replication, e.g. measurable or significant replication, occurs. For example, such mutation etc., can be any mutation etc., that reduces or inhibits or prevents the function of the origin of replication, for example reduces or inhibits (e.g. significantly reduces or inhibits) or prevents DNA replication at the origin of replication.

Features shared among bacterial origins of replication of different bacterial strains include an AT-rich DNA unwinding element (DUE) that facilitates DNA strand separation, and elements of nine nucleotides in length which serve as binding sites for DnaA (so-called “DnaA boxes”). In the process of DNA replication initiation, DnaA monomers bind to the DnaA boxes within the origin of replication. These DnaA monomers, while bound to the DnaA boxes, oligomerise to form a DnaA complex. The DnaA complex is sufficient both to unwind the DUE and assist in loading of DNA helicase (DnaB) onto the exposed single strands, which completes the formation of the bacterial pre-replication complex. Hence, it is generally understood in the art that the DUE and DnaA boxes are required for DNA replication initiation in bacteria.

Therefore, in embodiments, the irreversible (or permanent) inactivation of the origin of replication comprises mutation or other alteration or modification such that the binding of DnaA monomers to the origin of replication is reduced or prevented (e.g. significantly reduced or prevented), or the unwinding of the DUE is reduced or prevented (e.g. significantly reduced or prevented), or the binding of DNA helicases (e.g. binding of molecules of DnaB) is reduced or prevented (e.g. significantly reduced or prevented). In other words, the mutation, alteration or modification is such that DNA replication cannot be initiated or significantly initiated at the origin of replication or such that the origin of replication is inactive or essentially inactive. More specifically, the irreversible (or permanent) inactivation of the origin of replication can comprise mutation, etc., of at least one, at least two, at least three, at least four, or all, or up to 2, 3, or 4, DnaA boxes (or DnaA binding sites) of the origin of replication.

In embodiments, the irreversible (or permanent) inactivation of the origin of replication comprises mutation, etc., of at least 5, at least 6, at least 7, at least 8, at least 9, at least 10, at least 11, at least 12, at least 13, at least 14, at least 15 or all DnaA boxes of the origin of replication, or up to 5, 6, 7, 8, 9, 10, 11, 12, 13, 14, or 15 DnaA boxes, for example at least, or up to, 13 DnaA boxes. In embodiments, the irreversible (or permanent) inactivation of the origin of replication comprises removal (or deletion or elimination or excision) of at least, or up to, 1, 2, 3, 4, 5, 6, 7, 8, 9, 10, 11, 12, 13, 14, 15 or all DnaA boxes of the origin of replication, for example at least, or up to, 13 DnaA boxes. Preferably, the mutation is removal (or deletion or elimination or excision).

In embodiments, the product is a product (or any product or any product of interest) produced in or producible by a bacterial cell, for example produced in or producible by a two-stage bioprocess method of the art. Such products are thus products which it is desired to prepare or produce in elevated, measurable or significant quantities, e.g. are desirable, useful, industrial, or commercial products, or products of value, e.g. to industry or research. Hence, in embodiments, the product is a small molecule. Alternatively or in addition, the product may be a polypeptide or protein or protein complex (or protein of interest), for example an antibody, antigen, enzyme, growth factor or cytokine. Preferably, the product is a pharmaceutical compound, medicinal product, food additive, colouring compound, fragrance component, industrial chemical, fine chemical or biofuel. Preferably, the biofuel is alcohol, for example butanol or isobutanol.

Steps necessary to enable a bacteria to produce a desired product, e.g. inducing or allowing expression of genes necessary for product synthesis or regulation, would be well known to a person skilled in the art. For example, where the product is a small molecule, one or more enzymes may be expressed which can produce the product from a given substrate. Said substrate can be available homogenously or endogenously in the bacterial cell, for example where the substrate is an intermediate of a metabolic pathway within the bacterial cell. Alternatively or in addition, said substrate may be provided heterologously (e.g. by expression of a heterologous gene in a bacterial cell) or exogenously, for example by administration to the culture medium.

The production of a product may be enhanced by expression of regulatory proteins that increase the production of product, and/or by elimination of genes encoding enzymes and/or regulatory proteins that reduce the production of product. These steps are usually carried out by introducing changes into cellular DNA, e.g. by changes in the genome and/or in plasmids. Where the product is a heterologous protein, it is typically necessary to provide a nucleotide sequence encoding the heterologous protein. Optionally, nucleotide sequences encoding chaperone proteins and/or other auxiliary factors may also be provided in order to assist with folding and processing of the protein of interest. When testing the production of a product from a bacterial cell on small scale, the necessary heterologous genes are usually at first provided on plasmids or episomes. However, for an industrial scale it is almost always and preferably done by expression from the genome.

In embodiments the method of the present invention increases or improves the level or amount or concentration of product or product production (or product produced) by the bacterial cell, preferably in terms of titer, yield or productivity, or in terms of titer per cell, yield per cell, or productivity per cell. Preferably said increases are measurable or significant increases, for example are statistically or clinically significant. By way of example, a bacterial cell of the invention which lacks a functional origin of replication sequence (e.g. a bacterial cell of the invention whose origin of replication has been irreversibly (or permanently) inactivated) which can give rise to increases of at least, or up to, 1.5 fold, 2 fold, 2.5 fold, 3 fold, 3.5 fold, 4 fold, 4.5 fold, 5 fold, 6 fold, 7 fold, 8 fold, 9 fold or 10 fold, in levels of product production or product produced (preferably in terms of titer, yield or productivity, or in terms of titer per cell, yield per cell, or productivity per cell) or in activity of the enzyme or enzymes which produces said product or has a positive role in producing said product, is preferred. Any appropriate comparison can be used, for example an increase when compared to said levels observed from the same type of bacterial cell when irreversible (or permanent) removal of the origin of replication is not present or induced (e.g. an equivalent bacterial cell in which the origin of replication has not been irreversibly inactivated), or in comparison to said levels from a control strain, e.g. as described elsewhere.

The term “control strain” (or control cell) as used herein in general can refer to a bacterial strain (an equivalent bacterial strain) without or which does not have the irreversible (or permanent) removal of the origin of replication. For example, this term can refer to a bacterial strain (or bacterial cell) which differs from the bacterial cell of the invention in that it does not contain an origin of replication capable of irreversible (or permanent) inactivation, for example contains a native or wild-type or unmodified origin of replication, e.g. is a native or wild-type or unmodified strain or cell. Alternatively, the term “control strain” may indicate a bacterial strain or bacterial cell (for example the bacterial cell of the invention) when used in conditions where irreversible (or permanent) inactivation of the origin of replication is not present or triggered/induced (for example a switcher or switcher cell as defined herein, i.e. a modified bacterial cell whose origin of replication has been modified such that it can be irreversibly inactivated (i.e. is capable of being irreversibly inactivated) but which has not been irreversibly inactivated). Alternatively, the term “control strain” may indicate a bacterial strain or bacterial cell which possesses a modified origin of replication (e.g. such as that described above for a switcher cell), but wherein irreversible (or permanent) inactivation of the origin of replication cannot be induced, for example because the bacterial cell lacks an enzyme (or a functional form of said enzyme) which is responsible for catalysing the inactivation process (e.g. a site-specific recombinase, for example such control strains may contain a defective or non-functional recombinase, or lack an active recombinase).

Appropriate methods of measuring levels of product production or product produced (preferably in terms of titer, yield or productivity, or in terms of titer per cell, yield per cell, or productivity per cell) or activity of the enzyme(s) which produces said product would be well known to a person skilled in the art. Thus, in some embodiments of the invention, the method will involve the step of detecting or determining the amount or level (e.g. the concentration) of product produced by a bacterial strain (e.g. when determining titer or yield), and optionally measuring the time taken to produce said amount or level (e.g. the concentration) of product produced by the bacterial cell.

Thus, typically strains of the invention or for use in the methods of the invention will exhibit higher levels, sometimes significantly higher levels, of product production or product produced (preferably in terms of titer, yield or productivity, or in terms of titer per cell, yield per cell, or productivity per cell) when switched (i.e. after the origin of replication is irreversibly (or permanently) inactivated) than a control strain as described above, for example a switcher strain. If appropriate, the levels of product production can conveniently be measured or determined by methods known in the art. Thus, strains capable of higher (increased) levels, or significantly higher (increased) levels, of product production or product produced than a control (preferably in terms of titer, yield or productivity, or in terms of titer per cell, yield per cell, or productivity per cell), for example when assessed in vitro, form a yet further aspect of the invention.

The product produced by the bacterial cells may be secreted into the culture medium (or supernatant), or may be retained intracellularly. Therefore, the levels of production or product produced may be measured in terms of the level of product present in the culture medium (or supernatant), the level of product retained intracellularly (for example, this is measurable after standard protein extraction and purification), or both, as appropriate depending on the product concerned.

Viewed alternatively, strains capable of higher (increased) levels, or significantly higher (increased) levels, of product production or product produced when switched than a control strain (preferably in terms of titer, yield or productivity, or in terms of titer per cell, yield per cell, or productivity per cell), for example in the culture medium (or supernatant) of bacterial cells and/or intracellularly, for example when assessed in vitro, form a yet further aspect of the invention. For example, strains capable of higher (increased) levels, or significantly higher (increased) levels of product production or product produced in a subject when switched (preferably in terms of titer, yield or productivity, or in terms of titer per cell, yield per cell, or productivity per cell), for example higher (increased) local levels of product production or product produced in a subject when switched than a control strain, for example when assessed in vivo, form a yet further aspect of the invention, in particular where such an effect is observed when the strains are administered to a subject.

The bacterial cell or modified bacterial cell of the present invention may be administered to the subject in any appropriate manner conventional for therapy, e.g. therapy using bacterial cells, and for the treatment of the indicated diseases, including but not limited to oral, sublingual, transdermal, cutaneous, rectal, nasal, vaginal, or ocular administration, or administration via inhalation or via buccal administration or by injection. Additionally, the cells or compositions of the present invention may be formulated for parenteral administration, for example by injection or continuous infusion. The route of administration may be any route that effectively transports the bacterial cell or modified bacterial cell to the desired site without harming the recipient.

Preferably said higher levels or increases (in product production, or of product produced, or activity of the enzyme(s) which produces said product) in the bacterial strain are measurable or significant increases, for example are statistically or clinically significant. By way of example, strains which can give rise to increases of at least, or up to 10%, 20%, 30%, 40%, 50%, 60%, 70%, 80%, 90%, 100%, 150%, 200%, 250%, 300%, 350%, 400%, 450%, 500%, 550%, or higher, in levels of product or product production or product produced (preferably in terms of titer, yield or productivity, or in terms of titer per cell, yield per cell, or productivity per cell), for example local or in vitro levels of product production or product produced, or in levels of activity of the enzyme(s) which produces said product (for example when assessed in vitro), compared to the levels with a control as described above are preferred. Viewed alternatively, strains which can give rise to increases of at least, or up to, 1.5 fold, 2 fold, 2.5 fold, 3 fold, 3.5 fold, 4 fold, 4.5 fold, 5 fold, 6 fold, 7 fold, 8 fold, 9 fold, or 10 fold, in levels of product or product production or product produced (preferably in terms of titer, yield or productivity, or in terms of titer per cell, yield per cell, or productivity per cell), for example local or in vitro levels of product or product production or product produced, or in levels activity of the enzyme(s) which produces said product, compared to the levels with a control strain as described above are preferred. In vitro levels can conveniently be measured as described elsewhere herein, for example in bacterial cultures. Preferably, such increases are increases in extracellular (e.g. secreted) and/or intracellular levels of product (preferably in terms of titer or yield) or in activity of the enzyme(s) which produces said product, for example as measured in vitro, for example in the culture medium (or supernatant) of bacterial cultures, or intracellularly.

Appropriate methods for measuring the level of product or product production or product produced (preferably in terms of titer, yield or productivity, or in terms of titer per cell, yield per cell, or productivity per cell) or the activity of the enzyme(s) which produces said product are known to the skilled person and any of these can be used.

In embodiments the method of the present invention increases the level of product or product production or product produced by the bacterial cell (preferably in terms of titer, yield or productivity, or in terms of titer per cell, yield per cell, or productivity per cell). Preferably said increases are measurable or significant increases, for example are statistically or clinically significant. By way of example, it is preferred that the irreversible (or permanent) inactivation of an origin of replication in a bacterial cell (or the lack of a functional origin of replication sequence in a bacterial cell) when used in the methods of the present invention gives rise to fold increases or percentage increases as defined above, in levels of product production or product produced (preferably in terms of titer, yield or productivity, or in terms of titer per cell, yield per cell, or productivity per cell) or in activity of the enzyme(s) which produces said product. Any appropriate comparison can be used, for example an increase in levels (preferably in terms of titer, yield or productivity, or in terms of titer per cell, yield per cell, or productivity per cell) when compared to the levels observed in the same bacterial cell (or same type of bacterial cell) when an origin of replication in the bacterial cell is not inactivated (or is not irreversibly inactivated), or the levels (preferably in terms of titer, yield or productivity, or in terms of titer per cell, yield per cell, or productivity per cell) when compared to the levels with a control cell, e.g. a control cell as described elsewhere herein.

Optionally, the level of product or product production or product produced (preferably titer, yield or productivity) by the bacterial cell can be quantified per number of bacterial cells (or per bacterial cell population size or per bacterial cell population density). In other words, the level of product or product production or product produced by the bacterial cell (preferably titer, yield or productivity) can be quantified in a manner which takes into account the number of bacterial cells (or bacterial cell population size or bacterial cell population density) used to produce the product.

Appropriate methods of measuring the bacterial cell population size or the numbers of bacterial cells are well known in the art including by measurement of optical density of the bacterial cell population at 600 nm (OD₆₀₀).

In embodiments, the bacterial cell of the invention or the bacterial cell used in the methods of the invention retains the ability to produce product (or to be induced/stimulated to produce product) for a period of time after an origin of replication of the bacterial cell is irreversibly (or permanently) inactivated (preferably such ability is retained for a greater or significantly greater period of time compared to a control cell, e.g. a control cell as described elsewhere herein). The bacterial cell of the invention or the bacterial cell used in the methods of the invention may retain the ability to produce product (or to be induced/stimulated to produce product) for up to (or at least) 3 hours, up to (or at least) 4 hours, up to (or at least) 5 hours, up to (or at least) 6 hours, up to (or at least) 7 hours, up to (or at least) 8 hours, up to (or at least) 9 hours, up to (or at least) 10 hours, up to (or at least) 11 hours, up to (or at least) 12 hours, up to (or at least) 13 hours, up to (or at least) 14 hours, up to (or at least) 15 hours, up to (or at least) 20 hours, up to (or at least) 25 hours, or up to (or at least) 30 hours after an origin of replication of the bacterial cell is irreversibly (or permanently) inactivated or after the irreversible (or permanent) inactivation of the bacterial cell is triggered or induced, or after the step to irreversibly inactivate an origin of replication in the bacterial cell has been carried out. It is also preferred that at any particular time point the ability of the bacterial cell of the invention, or used in the methods of the invention to produce product, is greater, e.g. significantly greater, than an appropriate control cell as described elsewhere herein, e.g. compared to a switcher cell (i.e. a modified bacterial cell of the invention whose origin of replication has not been inactivated) or a cell in which irreversible inactivation of the origin of replication cannot take place, or an unmodified or wild-type cell.

Irreversible inactivation of the origin of replication in a bacterial cell should lead to a stop in cell division and growth. Once irreversible inactivation of the origin of replication in the bacterial cell is induced or triggered, the bacterial cell culture gradually reaches a distinct cell density plateau. The value at which cell density stops increasing (i.e. reaches a plateau) can be selected by tuning the starting cell density of the bacterial cell culture (for example, the cell density of the inoculated culture, and/or the dilution factor of the inoculated culture) and/or the cell density at which irreversible inactivation of the origin of replication is induced.

Hence, in embodiments the peak cell density (or maximum cell density, or maximal cell density, or cell density plateau) of a culture of the bacterial cell of the invention, or used in the methods of the invention, is at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, about 100% (or approximately the same, or the same), over 100%, at least 100%, at least 105%, at least 110%, about 110%, at least 115%, at least 120%, at least 125%, at least 130%, at least 135%, at least 140%, at least 145%, at least 150%, of the peak cell density of a control bacterial cell culture. Values “up to” these percentages are also provided.

Preferably, the peak cell density (or maximum cell density, or maximal cell density, or cell density plateau) of a culture of the bacterial cell of the invention, or used in the methods of the invention, is at least 70%, at least 75%, at least 80%, at least 85%, at least 90%, at least 95%, at least 100%, about 100% (or approximately the same, or the same), over 100%, at least 100%, at least 105%, at least 110%, about 110%, at least 115%, or at least 120% of the peak cell density of a control bacterial cell culture. Values “up to” these percentages are also provided.

Preferably, the peak cell density (or maximum cell density, or maximal cell density, or cell density plateau) of a culture of the bacterial cell of the invention, or used in the methods of the invention, is at least 90%, at least 95%, at least 100%, about 100% (or approximately the same, or the same) over 100%, at least 100%, at least 105%, at least 110%, or about 110%, of the peak cell density of a control bacterial cell culture. Values “up to” these percentages are also provided.

An appropriate control bacterial cell culture is as described elsewhere herein, and can for example be a culture of the bacterial cell of the invention wherein irreversible inactivation of the origin of replication is not induced (e.g. irreversible inactivation of the origin of replication does not or cannot take place).

In embodiments, the level of product produced or level of production by a bacterial cell or modified bacterial cell of the invention, or used in the methods of the invention, is higher where the peak cell density (or maximum cell density, or maximal cell density, or cell density plateau) of the bacterial cell culture or modified bacterial cell culture is closer to that of a control bacterial cell culture, for example at least 70% of the peak cell density etc., of a control bacterial cell culture as described above.

In embodiments, the method of the invention increases the yield or titer of the product produced by the bacterial cell, or increases the productivity of the bacterial cell.

The term “titer” means the amount or concentration of product produced during or at the end of the bioprocess. Hence, titer can be measured in units of “g/L” or in any other appropriate units.

The term “yield” means the mass of product produced per mass of substrate (e.g. carbon and energy source) provided to the bacteria for the production method or bioprocess. Hence, yield can be measured in units of “g/g” or in any other appropriate units.

The term “productivity” relates to how quickly the bacteria are producing the product in the bioprocess. Hence, productivity is the amount or concentration of product produced per unit time. Hence, productivity can be measured in units of “g L⁻¹”, i.e. grams per litre per hour or in any other appropriate units.

In embodiments, the product is a heterologous product.

The term “heterologous” is generally understood in the art. Hence a “heterologous product” in the context of the invention is a product that is not normally (i.e. not naturally or not natively) produced by the bacteria. Hence, for a bacterium to produce a heterologous product it may need to be modified genetically or to be exposed to a heterologous substrate. Hence “heterologous” can mean “exogenous”. The term “heterologous” contrasts with “homologous” and “endogenous”, which are also generally understood in the art.

In embodiments, the product is a homologous product.

The term “homologous” is generally understood in the art. Hence, the term “homologous” as used in the context of the invention is a product that is normally (i.e. naturally or natively) produced by the bacteria. Hence, for a bacterium to produce a homologous product, it may not need to be exposed to a heterologous substrate. Hence “homologous” can mean “endogenous”.

Where the product is a homologous or endogenous product, the bacterial cell may already produce the product before the origin of replication is irreversibly removed (i.e. before switching). In this instance, irreversible removal of the origin of replication can be used to increase the levels of product or to overexpress the product, e.g. to increase the titer, yield and/or productivity of the bacterial cell in producing the homologous or endogenous product, e.g. compared to a control bacterial cell, as discussed elsewhere herein.

In embodiments, irreversibly (or permanently) inactivating an origin of replication may be achieved through genetic (or nucleotide) modification, wherein the genetic (or nucleotide) modification comprises or consists of removal or modification or mutation of the full or complete nucleotide sequence of the origin of replication. Alternatively viewed, irreversibly (or permanently) inactivating an origin of replication may be achieved through removal of the full nucleotide sequence of the origin of replication. Alternatively viewed, irreversibly (or permanently) inactivating an origin of replication may be achieved by removing (or deleting or eliminating or excising) the full sequence of the origin of replication, i.e. by removing (or fully deleting or eliminating or excising) all of the origin of replication. In E. coli the ori sequence is called oriC, which is situated between mnmG and mioC genes in common laboratory strains (Messer, 2002). Thus, by way of example, removing (or deleting or eliminating or excising) all or part of the nucleotide sequence between these two genes can result in removal of the origin of replication and irreversible inactivation.

In another embodiment irreversibly (or permanently) inactivating an origin of replication may be achieved through genetic (or nucleotide) modification, wherein the genetic (or nucleotide) modification comprises or consists of removal or modification or mutation of at least part of the origin of replication, or only a part of the origin of replication. Alternatively viewed, irreversibly (or permanently) inactivating an origin of replication may be achieved by removing (or deleting or eliminating or excising) at least part of the origin of replication, or only a part of the origin of replication.

Embodiments where at least part or only a part of the origin of replication is modified or removed (e.g. partial removal etc.) encompass any removal or modification which results in loss or significant reduction of the ability of the origin of replication to function as an origin of replication, for example loss or significant reduction of the ability to initiate DNA replication at that site, or any removal or modification that results in irreversible inactivation of the origin of replication.

The term “removed” (or deleted or eliminated or excised) or similar as used herein means that the origin of replication (or a part thereof) has been removed (or deleted or eliminated or excised) from the polynucleotide in which it was contained so as to render the bacterial cell incapable (or with significantly reduced capability) of replicating the polynucleotide in which it was contained. Hence, an origin of replication (or a part thereof) can be considered “removed” (or deleted or eliminated or excised) if it is removed from the polynucleotide in which it was originally contained, for example from the bacterial chromosome. Hence, an origin of replication (or part thereof) which has been deleted from the polynucleotide in which it was contained or originally contained but nevertheless remains in the bacterial cell (e.g. as a linear sequence or circular sequence or plasmid or episome within the cell), can still be considered to be “removed”. Optionally, the term “removed” means removed from the bacterial cell such that it is no longer contained in the bacterial cell.

Removing (or deleting or eliminating or excising) or modification of at least part of the origin of replication, or only a part of the origin of replication (e.g. partial removal), may encompass the removal of multiple non-contiguous (or non-continuous or non-consecutive) sequences within the origin of replication, for example the modification (e.g. mutation) or removal of one or more, or two or more, etc., DnaA box sequences from the origin of replication, e.g. as described elsewhere herein. Alternatively, references to the “partial” removal of an origin of replication, or the removal of “part” of an origin of replication may encompass only the modification (e.g. mutation) or removal of a single contiguous (or continuous or consecutive) sequence in the origin of replication.

In embodiments, the genetic modification comprises or consists of partial removal of the nucleotide sequence of the origin of replication.

In embodiments, the partial removal (or partial deletion or partial elimination or partial excision) of the origin of replication comprises or consists of removal (or deletion or elimination or excision) of at least 5%, 10%, 15%, 20%, 25%, 30%, 35%, 40%, 45%, 50%, 55%, 60%, 65%, 70%, 75%, 80%, 85%, 90%, or 95%, or 100% of the nucleotide sequences of the DnaA boxes of the origin of replication. Preferably, the partial removal (or partial deletion or partial elimination or partial excision) of the origin of replication comprises or consists of removal (or deletion or elimination or excision) of at least one third (or at least 33%), at least one half (or at least 50%), at least two thirds (or at least 67%) or all of the nucleotide sequence of the DnaA boxes of the origin of replication. Values “up to” these percentages are also provided.

In embodiments, the partial removal (or partial deletion or partial elimination or partial excision) of the origin of replication comprises or consists of removal (or deletion or elimination or excision) of at least 5%, 10%, 15%, 20%, 25%, 30%, 35%, 40%, 45%, 50%, 55%, 60%, 65%, 70%, 75%, 80%, 85%, 90%, or 95%, or 100% of the nucleotide sequence of the DNA unwinding element (DUE) of the origin of replication. Preferably, the partial removal (or partial deletion or partial elimination or partial excision) of the origin of replication comprises removal (or deletion or elimination or excision) of at least one third (or at least 33%), at least one half (or at least 50%), at least two thirds (or at least 67%), or all of the nucleotide sequence of the DNA unwinding element of the origin of replication. Values “up to” these percentages are also provided.

In embodiments, the partial removal (or partial deletion or partial elimination or partial excision) of the origin of replication preferably comprises deletion of at least one third (or at least 33%), at least one half (or at least 50%), at least two thirds (or at least 67%) or all of the nucleotide sequence of the origin of replication. Preferably, the partial removal (or partial deletion or partial elimination or partial excision) of the origin of replication comprises removal (or deletion or elimination or excision) of at least 5%, 10%, 15%, 20%, 25%, 30%, 35%, 40%, 45%, 50%, 55%, 60%, 65%, 70%, 75%, 80%, 85%, 90%, or 95%, or 100% of the nucleotide sequence of the original of replication. Values “up to” these percentages are also provided.

In embodiments, the method of the invention comprises:
(i) providing a bacterial cell capable of producing or producing said product;
(ii) irreversibly (or permanently) inactivating an origin of replication in the bacterial cell; and, optionally,
(iii) inducing production of the product in the bacterial cell.

Appropriate techniques for carrying out step (ii) would be well known to a person skilled in the art and any of these may be used. Preferred techniques, e.g. involving the use of recombination or site-specific recombination are described elsewhere herein.

In embodiments, steps (ii) and (iii) of the method of the invention take place simultaneously or sequentially. If sequentially, steps (ii) and (iii) can be carried out in any appropriate order.

In embodiments, the bacterial cell exists within a bacterial cell population. Indeed, in all embodiments of the invention, the bacterial cells are typically part of, or comprise or consist of, a bacterial cell population.

In preferred methods of the invention steps (ii) and/or (iii) of the method of the invention take place during the exponential growth phase of the bacterial cell population. The appropriate time point during the exponential growth phase to carry out steps (ii) and/or (iii) can readily be determined, e.g. for desired or maximum product production. For example, in some embodiments, it may be beneficial to carry out step (ii), in other words the activation of the switch, at the beginning (or in the first half) of the exponential growth phase where the cell density is low (or relatively low), or, in some embodiments, at a later part (e.g. in the second half) of the exponential growth phase where the cell density is high (e.g. higher or significantly higher than at the beginning of the exponential growth phase). In many embodiments, the optimal timing for the switch largely depends on the final (or maximum) cell density it is desired to achieve. If the desired final cell density is high then the switch will likely need to be induced later in the exponential growth phase, whereas if the desired final cell density is low (or does not need to be so high) then the switch can be induced early in the exponential growth phase.

It is well understood in the art that the growth of bacteria in culture can typically be modelled with four different phases: (1) lag phase, (2) exponential phase, (3) stationary phase, and (4) death phase. During the lag phase bacteria adapt to culture conditions, and minimal or no cell division occurs. During the exponential phase, cell division occurs and so the number of cells doubles with each consecutive time period. The stationary phase is reached due to a growth-limiting factor, for example the depletion of an essential nutrient. During the stationary phase the rate of cell growth matches the rate of cell death (for example, both can be effectively non-existent), i.e. the number of cells remains approximately the same. During or at the death phase, more bacteria die, and so the number of (living) cells decreases. These typical phases can for example be seen in FIGS. 3, 4 and 5 herein.

It is understood in the art that the time at which the exponential growth phase begins can either be predicted/known in advance (e.g. through knowledge of the growth pattern of the strain in question under specific conditions/inoculation levels) or by regularly monitoring growth, for example by measurement of optical density of the bacterial cell culture at 600 nm (OD₆₀₀).

The terms “switch” and “switching” as used herein refer to the process or step of irreversible (or permanent) inactivation of an origin of replication in a bacterial cell.

The terms “switch” and “switching” as used herein can refer to the process of a bacterial cell changing from growth stage to production stage, for example in a two-stage bioprocess method. In other words, the term “switch” and “switching” as used herein can refer to the process of a bacterial cell changing from: (i) a state (e.g. a metabolic state) wherein growth (e.g. cell growth, multiplication and/or increasing biomass) is favoured over product production, preferably wherein there is no product production; to (ii) a state (e.g. a metabolic state) wherein product production is favoured over growth (e.g. cell growth, multiplication and/or increasing biomass), preferably wherein there is no growth (e.g. cell growth, multiplication and/or increasing biomass).

In the methods of the present invention, the process of a bacterial cell changing from growth stage to production stage may be induced by (or may be caused by, or may be concomitant with) the irreversible (or permanent) inactivation of the origin of replication of the bacterial cell. Surprisingly such inactivation can provide a convenient way of stopping or significantly reducing the growth of bacterial cells. In addition, such inactivation can provide a convenient way to switch bacterial cells to a state wherein product production is favoured over growth (e.g. cell growth, multiplication and/or increasing biomass), preferably wherein there is no growth (e.g. cell growth, multiplication and/or increasing biomass). Thus, in the methods of the invention, such a step of irreversibly inactivating an origin of replication is preferably accompanied by product production (desired product production), e.g. endogenous product production or a step of inducing production of the product (desired product) at an appropriate time point.

Similarly, a “switcher” or “switcher cell” or “switcher strain” is a bacterial cell comprising an origin of replication, wherein the bacterial cell has been modified such that the origin of replication can be irreversibly (or permanently) inactivated. The term “switched” or “switched cell” or strain as used herein means a cell whose origin of replication has been irreversibly (or permanently) inactivated.

Switching, or the irreversible (or permanent) inactivation of the origin of replication, can be induced or carried out at any appropriate time point.

In embodiments, switching, or the irreversible (or permanent) inactivation of the origin of replication, is induced or carried out during the exponential growth phase of the bacterial cell population. For example, the irreversible (or permanent) inactivation of the origin of replication may be induced or carried out at the beginning, or approximately at the beginning, or immediately after the beginning, or shortly after the beginning, of the exponential growth phase of the bacterial cell population. In other words, the inactivation of the origin may for example be induced or carried out in the first half of the exponential growth phase, or at a time point at or before a cell density of 50% of the maximal cell density (or OD) is achieved. In other embodiments, switching, or the irreversible (or permanent) inactivation of the origin of replication, is induced or carried out later during the exponential growth phase of the bacterial cell population where the cell density is higher, for example in the second half of the exponential growth phase, or at a time point after a cell density of 50% of the maximal cell density (or OD) is achieved.

In an alternative embodiment, the switching, or irreversible (or permanent) inactivation of the origin of replication, may be induced or carried out before the exponential growth phase of the bacterial cell population. For example, the irreversible (or permanent) inactivation of the origin of replication may be induced or carried out immediately (or shortly) before the beginning of the exponential growth phase of the bacterial cell population.

Hence switching, or the irreversible (or permanent) inactivation of the origin of replication, is induced or carried out either at a predicted or convenient or suitable time after inoculation of bacteria into the culture medium, or after, e.g. immediately or soon after, the cell density (optical density) of the bacterial culture reaches a certain or desired threshold level.

Preferably, the threshold optical density level after, e.g. immediately after, which switching (i.e. irreversible inactivation of the origin of replication) should be initiated or induced is at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the peak cell density (or maximum cell density, or maximal cell density, or cell density plateau) of the bacterial cell culture after switching, or of a control bacterial cell culture, e.g. of the peak cell density of the bacterial cell culture in the absence of switching.

Preferably, the threshold optical density level immediately after which switching (i.e. irreversible inactivation of the origin of replication) should be initiated or induced is between 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, or 85 to 90% of the peak cell density (or maximum cell density, or maximal cell density, or cell density plateau) of the bacterial cell culture after switching, or of a control bacterial cell culture, e.g. of the peak cell density of the bacterial cell culture in the absence of switching.

The optical density level for the peak or maximum etc., cell density as discussed above and elsewhere herein can be predetermined but will vary for different bacterial cells under different conditions, for example different growth conditions such as media and temperature, or the production of different products. Thus, using a percentage of maximum cell density that is achieved under chosen or desired conditions is particularly convenient to determine when switching (or irreversible inactivation of the origin of replication) should be initiated or induced.

The control bacterial cell culture can for example be a culture of the bacterial cell of the invention wherein irreversible inactivation of the origin of replication is not induced (i.e. irreversible inactivation of the origin of replication does not or cannot take place).

Production of the desired product may take place at any appropriate time point which could readily be determined by a person skilled in the art.

In embodiments, production of the desired product may be initiated or induced (for example, by inducing production and/or activation of the enzyme(s) or metabolic pathway(s) responsible for generation of the desired product or otherwise inducing production of the product, e.g. where the product is a protein, by inducing expression of the gene or nucleic acid sequence encoding the product) at the same time as switching or the irreversible (or permanent) inactivation of the origin of replication, is induced or carried out (or at approximately the same time as switching or origin of replication inactivation, or immediately before switching or origin of replication inactivation, or immediately after switching or origin of replication inactivation, or shortly before or after switching or origin of replication inactivation).

Alternatively, the timing of initiation or induction of production of the product (desired product) may not necessarily be tied to the time of induction of switching or origin of replication inactivation, and can be carried out at any appropriate time point providing the bacterial cells are capable of producing measurable or significant levels of the product (desired product). For example, in embodiments, production of product is initiated or induced (for example, by inducing production and/or activation of the enzyme(s) or metabolic pathway(s) responsible for generation of the desired product or otherwise inducing production of the product, e.g. where the product is a protein, by inducing expression of the gene or nucleic acid sequence encoding the product) during (or approximately at the beginning, or immediately before the beginning, or immediately after the beginning, or shortly before the beginning, or shortly after the beginning of) the exponential growth phase of the bacterial cell population. In other words, product production may for example be induced or carried out in the first half of the exponential growth phase, or at a time point at or before a cell density of 50% of the maximal cell density (or OD) is achieved. In other embodiments, product production may for example be induced or carried out later during the exponential growth phase of the bacterial cell population where the cell density is higher, for example in the second half of the exponential growth phase, or at a time point after a cell density of 50% of the maximal cell density (or OD) is achieved.

Alternatively, production of product may need not be tied to the timing of the exponential phase. For example, the product may be constitutively or endogenously produced (in which case no step of initiation or induction is necessarily required), or initiation or induction may be started before or after the beginning of the exponential phase of bacterial cell culture growth.

In alternative embodiments, production of product is initiated or induced at other time points during the exponential growth phase or even during the stationary phase. Indeed, it is a surprising advantage of the methods of the present invention that the bacterial cells can still be initiated or induced to produce product (desired product) whilst the bacterial cells are in the stationary phase of growth.

Hence product production may be initiated or induced either at a predicted or convenient or suitable time after inoculation of bacteria into the culture medium, or after, e.g. immediately or soon after, the optical density of the bacterial culture reaches a certain or desired threshold level.

Preferably, the threshold optical density level immediately after which product production should be initiated or induced is at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the peak cell density (or maximum cell density, or maximal cell density, or cell density plateau) of the bacterial cell culture after switching, or of a control bacterial cell culture, e.g. of the peak cell density of the bacterial cell culture in the absence of switching.

Preferably, the threshold optical density level immediately after which product production should be initiated or induced is between 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, or 85 to 90% of the peak cell density (or maximum cell density, or maximal cell density, or cell density plateau) of the bacterial cell culture after switching, or of a control bacterial cell culture, e.g. of the peak cell density of the bacterial cell culture in the absence of switching.

The optical density level for the peak or maximum etc., cell density as discussed above and elsewhere herein can be predetermined but will vary for different bacterial cells under different conditions, for example different growth conditions such as media and temperature, or the production of different products. Thus, using a percentage of maximum cell density that is achieved under chosen or desired conditions is particularly convenient to determine when product production should be initiated or induced.

The control bacterial cell culture can for example be a culture of the bacterial cell of the invention wherein irreversible inactivation of the origin of replication is not induced (i.e. irreversible inactivation of the origin of replication does not or cannot take place).

It will be appreciated that in some embodiments it is not necessary for the user to induce production of the product and that this step can be optional, for example because induction or production of the product takes place automatically in culture at a certain time point. This may be the case for example where production of the product is linked to a promoter which is sensitive to internal stimuli or internal conditions. For example, the promoter may be induced when the bacterial cell culture enters a specific phase of growth, for example as in the case of PCP_2836 promoter (Ma et al., 2018), phoPR promoter (Paul et al., 2004) and P170 promoter (Madsen et al., 1999). Preferably, the promoter may be induced when the bacterial cell culture enters the exponential growth phase, or during the exponential growth phase.

Other preferred settings include the use of auto-induction media (for example as described in Studier et al. 2005, Protein Expression and Purification, 2005, 41:207-234) and self-inducing systems (for example SILEX, as described in Briand et al. 2016, Scientific Reports, 6:33037).

Alternatively, it may not be necessary for the user to induce production of the product in the bacterial cell if the product is produced constitutively or continuously or constantly, e.g. under the control of a constitutive or endogenous promoter, for example an endogenous product (although equally constitutive or continuous or constant production can be applied to the production of a heterologous product). In this case, irreversible inactivation of the origin of replication in the bacterial cell can allow an increase or enhancement in the production or levels of desired product, e.g. to increase the titer, yield and/or productivity of the bacterial cell in producing said product.

In embodiments, the bacterial cell comprises a gene (or nucleic acid molecule) encoding the product or encoding an enzyme required in order to produce the product. Such genes can be homologous (endogenous) or heterologous.

In embodiments, the gene is operatively linked to a promoter. Such promoters can control the expression of the product or enzyme, e.g. the homologous (endogenous) or heterologous product or enzyme. Thus, in some embodiments such promoters can be endogenous promoters (e.g. promoters that are native to the gene or bacterial cell) or can be constitutive promoters in order to enable continuous or constant expression. Alternatively, such promoters can be inducible or heterologous promoters, which can for example be used to enable the induction of the production of the product encoded by the gene in the bacterial cell at a desired time point.

Appropriate inducible promoters would be well known and described in the art, and any of these could be used. For example, in embodiments, the promoter can be pH-sensitive, light-sensitive, temperature-sensitive, or chemically-sensitive. Examples and further details of such promoters and systems are discussed elsewhere herein. An exemplary system is also shown in the Examples.

In other embodiments, expression of the gene or the promoter may be induced or controlled by targeted proteolysis or by other appropriate techniques such as CRISPR interference (CRISPRi).

The concept of CRISPR interference (CRISPRi) is well known in the art and is described for example in Lei et al. 2013 (Cell 152, 1173-1183). In CRISPRi, a catalytically inactive version of Cas9 is targeted to a specific DNA sequence using sgRNA with a specific sequence. The catalytically inactive Cas9 binds to the target DNA region (for example a promoter region) to block RNA polymerase binding and transcript elongation.

Hence, in embodiments of the bacterial cell wherein expression of the gene or the promoter is induced or controlled by CRISPR interference (CRISPRi), the bacterial cell comprises a gene encoding a catalytically inactive Cas9 and an sgRNA for targeting the catalytically inactive Cas9 to the gene or promoter for producing the desired product such that expression of the gene or promoter is inhibited.

In an alternative view of the method of the invention, or in another aspect, the invention provides a method of irreversibly (or permanently) stopping growth of a bacterial cell without impairing the metabolic activity of the bacterial cell, the method comprising irreversibly (or permanently) inactivating an origin of replication in the bacterial cell. Thus, the methods of the invention also provide a method of stopping growth of a bacterial cell without impairing the metabolic activity of the bacterial cell.

By the phrase “stopping growth of a bacterial cell without impairing the metabolic activity of the bacterial cell”, it is meant that the bacterial cell is unable to engage in DNA replication but is otherwise substantially unaffected, i.e. the metabolic pathways of the bacterial cell, in particular the pathways associated with desired product production, e.g. product or enzyme gene expression, are left intact or functional (or at least substantially intact or functional). This is in contrast to two stage bioprocess methods of the art, where changing from growth stage to production stage required the targeted downregulation or inactivation of one or more specific metabolic pathways (for example, through targeted inhibition of metabolic pathway enzymes or targeted inhibition of the expression of genes encoding said enzymes) and/or the targeted upregulation or activation of other specific pathways (for example, through targeted activation of metabolic pathway enzymes or targeted induction of the expression of genes encoding said enzymes).

In another aspect, the invention provides a bacterial cell comprising an origin of replication, wherein the bacterial cell has been modified such that the origin of replication can be irreversibly (or permanently) inactivated. Such aspects therefore provide modified bacterial cells.

Various methods of irreversibly inactivating genetic elements are known in the art and the skilled person would understand how to apply these methods to the inactivation of an origin of replication. For example, said bacterial cell may comprise a chromosome which has been genetically modified such that the origin of replication in that chromosome can be rendered incapable of acting as an origin of replication, e.g. incapable of acting as a site of DNA replication initiation. The inactivation may be executed by mutation or deletion of the origin of replication. Preferably, the inactivation may be induced or controlled for example through control of an agent responsible for or involved in the mutation or deletion event. For example, the inactivation may induced or controlled via a promoter which is operatively linked to a gene encoding an agent responsible for or involved in the mutation or deletion event. The irreversible inactivation of the origin of replication can then be triggered by induction of said promoter. Thus, preferred promoters for this are inducible promoters.

A preferred method of irreversibly inactivating an origin of replication in accordance with the present invention is through use of homologous recombination to remove or delete all or part of the origin of replication nucleotide sequence. For example, the chromosome containing the origin of replication can be genetically modified to include appropriate sites to initiate homologous recombination, e.g. site-specific recombination sites for recognition by site-specific recombinases, which are positioned and orientated to allow the removal or deletion of all or part of the origin of replication nucleotide sequence. The site-specific recombinases can then be provided to the bacterial cell, conveniently by expressing them within the bacterial cell. This expression ideally needs to be controlled, preferably tightly controlled, so that expression of the recombinases can be induced at the required or appropriate time point when it is desired to trigger homologous recombination (or switching or origin inactivation). Conveniently this can be done by placing expression of the recombinases under the control of an inducible promoter.

In embodiments of a bacterial cell comprising an irreversibly inactivatable origin of replication, or methods using said bacterial cell, the origin of replication can be removed (or removable) using standard techniques in the art, for example using recombinases, for example using site-specific recombination or homologous recombination, for example using a serine recombinase or tyrosine recombinase system, or the phage lambda Red recombinase system (see for example Progress in Biophysics and Molecular Biology 147 (2019) 33e46).

In embodiments of a bacterial cell comprising an irreversibly inactivatable origin of replication, or methods using said bacterial cell, the origin of replication may be removed (i.e. switching may occur) by replacing the origin of replication with another sequence, for example by homologous recombination. In such embodiments, the replacement sequence should be a sequence which does not enable DNA replication initiation to be retained at that site after switching. Put another way, the replacement sequence should not be a sequence which enables DNA replication initiation from that site to be retained. For example, the irreversibly inactivatable origin of replication should not be replaced with another origin of replication which is functional in said bacterial cell.

In embodiments of a bacterial cell comprising an irreversibly inactivatable origin of replication, or methods using said bacterial cell, the origin of replication may be removed (i.e. switching may occur) by replacing the origin of replication with another sequence, for example by homologous recombination. In such embodiments, the replacement sequence should be a sequence which does not enable DNA replication initiation to be retained at that site after switching. Put another way, the replacement sequence should not be a sequence which enables DNA replication initiation from that site to be retained. For example, the irreversibly inactivatable origin of replication should not be replaced with another origin of replication which is functional in said bacterial cell.

Other types of genome editing can equally be used in order to irreversibly (or permanently) inactivate the origin of replication. Possible candidates include CRISPR/Cas systems (either alone or in combination with another recombinase system such as the phage lambda Red recombinase system) or the use of any other type of enzyme that irreversibly (or permanently) alters, e.g. removes or excises, the origin of replication sequence in the bacteria, for example in the bacterial chromosome, and leaves said origin of replication incapable of initiating DNA replication as discussed elsewhere herein.

Thus, an exemplary modified bacterial cell might contain an origin of replication flanked by recombination sites, e.g. site-specific recombination sites, to enable all or part of the origin of replication sequence to be irreversibly (or permanently) inactivated, for example by allowing the replication origin to be removed or excised. Such recombination sites could readily be introduced using standard techniques in the art.

A bacterial cell comprising an origin of replication, wherein the bacterial cell has been modified such that the origin of replication can be irreversibly (or permanently) inactivated, can be generated using standard techniques in the art, for example using recombinases, for example using homologous recombination, for example using the phage lambda Red recombinase system (see for example Progress in Biophysics and Molecular Biology 147 (2019) 33e46). Such homologous recombination using recombinases can for example be carried out using a vector comprising an origin of replication flanked by site-specific recombination sites, wherein the site-specific recombination sites are themselves flanked by (i.e. nested within) nucleotide sequences homologous to the corresponding sequence in the chromosome of the bacterial cell of interest.

In the process of generating a bacterial cell comprising an irreversibly inactivatable origin of replication, the original (i.e. native, natural, wild-type or endogenous) origin of replication of the starting bacterial cell may be replaced with a different origin of replication. For example, the inserted (i.e. irreversibly inactivatable) origin of replication may have a different sequence (e.g. the origin of replication may be from a different bacterial species or strain, or may be an exogenous sequence) as compared to the original (i.e. native, natural, wild-type or endogenous) origin of replication of the starting bacterial cell.

Similarly, in the methods of producing product and other aspects of the invention described herein, the origin of replication which is irreversibly inactivated (or irreversibly inactivatable) in a bacterial cell may have a different sequence (e.g. the origin of replication may be from a different bacterial species or strain, or may be an exogenous sequence) as compared to the original (i.e. native, natural, wild-type or endogenous) origin of replication of the bacterial cell used.

In embodiments, the origin of replication is flanked by site-specific recombination sites (e.g. site-specific recognition and cleavage sites) for recognition and cleavage by a site-specific recombinase.

A relevant site-specific recombinase can then bind and cleave said site so as to facilitate or enable DNA recombination.

In embodiments, the origin of replication is flanked by site-specific recombination sites which are recognizable by a site-specific recombinase enzyme (e.g. two site-specific recombination sites may be present, one positioned 5′ to the origin of replication and one positioned 3′ to the origin of replication).

In embodiments, the site-specific recombination sites have different sequences, for example attB and attP sites. Alternatively, the site-specific recombination sites have the same sequence (i.e. the site-specific recombination sites are identical), for example loxP sites. The appropriate site-specific recombination sites are chosen depending on the recombinase enzyme being used and would be readily derivable for a person skilled in the art.

In embodiments, each site-specific recombination site is placed at an appropriate distance from one end (either the 5′ or 3′ end) of the origin of replication, such that they flank the nucleotide region it is desired to remove, including all or part of the origin of replication, and are appropriately spaced such that homologous recombination can successfully occur.

Most bacteria and bacterial cells contain only a single origin of replication (e.g. a single chromosomal origin of replication), although in some cases the origin of replication comprises more than one stretch of nucleotides, e.g. the origin is not present in a single contiguous nucleotide sequence.

For example, in Bacillus subtilis, the origin of replication is bipartite, i.e. the origin of replication consists of two nucleotide stretches, separated by an intervening region (the dnaA gene) which is not part of the origin of replication. Hence, in the context of Bacillus subtilis and other bacterial strains possessing a bipartite origin of replication, the phrase “the origin of replication is flanked by site-specific recombination sites” or similar can mean that the recombination sites flank one or both nucleotide stretches of the origin of replication.

Where a bacterial cell possesses multiple origins of replication (e.g. multiple chromosomal origins of replication), an increase in product production (preferably in terms of titer, yield or productivity) in accordance with the present invention may be induced by irreversibly (or permanently) inactivating one, at least one or all of the origins of replication (e.g. chromosomal origins of replication) in the bacterial cell. Hence, in embodiments, where the origin of replication is multipartite, i.e. consists of two separate nucleotide stretches (i.e. is bipartite) or more (e.g. is tripartite), the origin of replication may be irreversibly (or permanently) inactivated by removal (or deletion or elimination or excision) of one, or both, or all of the nucleotide stretches of the origin or replication.

A bacterial cell may comprise one or more origins of replication on extra-chromosomal genetic material such as plasmids or episomes. In such cases, it is sufficient to irreversibly (or permanently) inactivate only the chromosomal origin of replication to facilitate switching.

Therefore, in embodiments, switching comprises or consists of (or is induced by) irreversibly (or permanently) inactivating a chromosomal origin of replication in the bacterial cell. Preferably, switching comprises or consists of (or is induced by) inactivating only one origin of replication in the cell, preferably only one chromosomal origin of replication in the cell.

The term “recombinase” as used herein encompasses any site-specific enzyme capable of DNA manipulation by excision, insertion, inversion or translocation. Hence, the term “recombinase” encompasses not only recombinases but also integrases, invertases and resolvases.

The term “site-specific recombinase” (or site-specific integrase or site-specific invertase or site-specific resolvase) as used herein means an enzyme which catalyzes DNA exchange reactions between target site sequences that are specific to that recombinase. Hence, the site-specific recombinases (and site-specific recombinase sites) as used herein can facilitate removal (or deletion or elimination or excision) of at least part, and preferably all, of the origin of replication of the bacterial cell.

The relative orientation of site-specific recombination sites, e.g. a pair of site-specific recombination sites, determines the outcome the recombination event. The term “orientation” (or direction or directionality) of the site-specific recombination sites as used herein is generally understood in the art and relates to the direction that the site-specific recombinase site is provided in the polynucleotide. For example, where site-specific recombination sites, e.g. a pair of site-specific recombination sites, on the same polynucleotide have opposite orientations, inversion of the flanked sequence within the polynucleotide may occur (for example with a pair of loxP sites (in the Cre-lox system), or with a pair consisting of an attP site and an attB site (for example as found in serine recombinase systems). In contrast, where two site-specific recombination sites have the same orientation (or parallel orientation), excision of the flanked sequence from the polynucleotide may occur.

Hence, in embodiments the site-specific recombination sites flank (i.e. are positioned either side of) the origin of replication in the bacterial chromosome in an orientation such that site-specific recombination leads to excision of the origin of replication from the bacterial chromosome. In embodiments, the site-specific recombination sites may be positioned in the same orientation on the polynucleotide. Alternatively, the site-specific recombination sites may be positioned in opposite orientations on the polynucleotide (i.e. wherein one site is in “sense” orientation and the other site is in “anti-sense” orientation).

In embodiments, the site-specific recombination sites comprise site-specific recombination sites for recognition by a serine recombinase. Preferably, the serine recombinase is yO, Bxb1, φC31 or TP901. However, there are many different serine recombinases described in the art and experimentally and more than 4000 new ones predicted by bioinformatics (Yang et al., 2014). Any of these may be used.

In embodiments, the site-specific recombination sites comprise site-specific recombination sites for recognition by a serine integrase.

The mechanism of recombination by serine site-specific integrases (a subfamily of serine site-specific recombinases) is generally understood in the art. Serine site-specific integrases catalyse recombination of attP and attB sites, generating attL and attR sites. The outcome of recombination of attP and attB sites is dependent on the orientation of the site-specific recombination sites. Recombination of an attP site and an attB site having the same orientation and located on the same polynucleotide results in excision of the flanked sequence from the polynucleotide, leaving an attR or attL site in the remaining polynucleotide In contrast, recombination of an attP site and an attB site with opposite orientation results in DNA sequence inversion.

Hence, in embodiments the serine site-specific recombination sites flank (i.e. are positioned either side of) the origin of replication in the bacterial chromosome in an orientation such that recombination (when catalysed by an appropriate enzyme) leads to excision of the origin of replication from the bacterial chromosome. Preferably, the serine site-specific recombination sites may be positioned in the same orientation on the polynucleotide. Alternatively, the serine site-specific recombination sites may be positioned in opposite orientations on the polynucleotide (i.e. wherein one site is in “sense” orientation and the other site is in “anti-sense” orientation).

In embodiments, the site-specific recombination sites comprise site-specific recombination sites for recognition by a tyrosine recombinase, preferably wherein the tyrosine recombinase is Cre, Dre, Flp, KD, B2 or B3.

Examples of site-specific recombinase technologies suitable for use in the present invention include serine site-specific recombination, tyrosine site-specific recombination, Cre-Lox recombination, FLP-FRT recombination, and homologous recombination.

In embodiments, the site-specific recombination sites comprise or consist of a pair of site-specific recombination sites.

The term “pair” of sites as used herein refers to two sites which are recognised by the same site-specific recombinase enzyme and wherein both sites must present for the recombination event to occur. Hence, an example of a pair of site-specific recombination sites is an attP site and an attB site (e.g. for recognition by a serine recombinase/integrase), or two loxP sites (e.g. for recognition by Cre recombinase).

Other examples of pairs of site-specific recombination sites would be known in the art.

Hence, other examples of pairs of site-specific recombination sites are: two FRT sites (e.g. for recognition by Flp recombinase); two rox sites (e.g. for recognition by Dre recombinase); two KD recombinase target (KDRT) sites (e.g. for recognition by KD recombinase); two B2 recombinase target (B2RT) sites (for recognition by B2 recombinase); or two B3 recombinase target (B3RT) sites (for recognition by B3 recombinase).

Thus, a “pair” of sites as described herein can be two different sites (with two different sequences) or two identical sites (with the same sequences) depending on the recombinase enzyme being used.

It is possible that a modified bacterial cell, comprising an irreversibly (or permanently) removable origin of replication, could undergo changes which inadvertently render the origin of replication non-removable (i.e. no longer removable). Loss of origin of replication removability may be caused, for example, by mutation of one or more site-specific recombination sites, or by mutation of one or more genes encoding the site-specific recombinase(s) specific for said sites.

Hence, it may be beneficial to provide a bacterial cell wherein the origin of replication is flanked by two, three or four (or at least, or up to, two, three, or four) pairs of site-specific recombination sites, preferably two pairs of site-specific recombination sites. Without wishing to be bound by theory, the probability of such bacterial cells losing the ability to irreversibly inactivate their origin of replication is reduced as compared to bacterial cells wherein the origin of replication is flanked by only one pair of site-specific recombination sites.

Hence, in embodiments, the site-specific recombination sites comprise or consist of two or more pairs, preferably two pairs, of site-specific recombination sites.

Alternatively viewed, the site-specific recombination sites comprise or consist of three, four, five or six pairs of site-specific recombination sites.

The probability of bacterial cells of the invention losing the ability to irreversibly inactivate their origin of replication decreases as the number of pairs of site-specific recombination sites is increased. Hence, for example, the probability of bacterial cells having three pairs of site-specific recombination sites losing the ability to irreversibly inactivate their origin of replication is lower than the probability of bacterial cells having two pairs of site-specific recombination sites losing the ability to irreversibly inactivate their origin of replication, and so on.

Where there are two or more pairs of site-specific recombination sites flanking the origin of replication, the pairs of site-specific recombination sites may be provided in any order along the chromosome providing the order is such so as to allow successful recombination to occur.

In embodiments, where two pairs of site-specific recombination sites are present, one pair of site-specific recombination sites is nested within the other, i.e. one pair of recombination sites is flanked by the other pair of recombination sites. Hence, for two pairs of recombination sites A and B, the sites are arranged along the polynucleotide in the order A-B-B-A, or in the order B-A-A-B.

Alternatively, the site-specific recombination sites from each pair are provided alternately along the polynucleotide, such that neither pair is nested within the other pair; in other words, for two pairs of recombination sites A and B, the sites are arranged in the polynucleotide in the order A-B-A-B, or in the order B-A-B-A.

The individual components of the two or more pairs of recombination sites are spatially arranged in such a way that recombination can occur. Thus, in a preferred embodiment, an appropriate number of nucleotides separate the adjacent A and B sites (i.e. the sites from each pair which are adjacent). In an alternative preferred embodiment, no nucleotides separate the adjacent A and B sites (i.e. the adjacent sites from each pair); in other words, the adjacent A and B sites (i.e. the adjacent sites from each pair) form one contiguous sequence.

The embodiments provided in the above paragraphs apply to further sets of pairs of site-specific recombination sites, for example three site-specific recombination sites, mutatis mutandis.

In embodiments, the bacterial cell of the invention further comprises a gene encoding a site-specific recombinase.

In embodiments, the gene encoding the site-specific recombinase is operatively linked to a promoter.

The site-specific recombinase is selected appropriately such that it can act with the chosen site-specific recombination sites. Thus, in embodiments where two or more pairs of recombination sites are used, the bacterial cell may further comprise a gene encoding a site-specific recombinase for each of the pairs of recombination sites, as appropriate, e.g. genes encoding 2 or more recombinases, e.g. 3, 4, 5 or 6 recombinases, as appropriate. In addition, the genes encoding each of the site-specific recombinase enzymes may be operatively linked to the same or different promoters. In addition, the genes encoding each of the site-specific recombinase enzymes may be on the same or different nucleic acid sequences, e.g. on the same or different extra-chromosomal element, e.g. plasmid or construct.

The term “promoter” as used herein takes its art recognised meaning and is generally understood to mean a sequence of DNA to which proteins bind in order to control, initiate and/or inhibit transcription of the DNA sequence downstream of the promoter. Where it is stated that the gene encoding the recombinase enzyme is “operatively linked” to a promoter, it is meant that the promoter is in a correct functional location and/or orientation in relation to the gene to control transcription of the sequence connected to the gene through the operation of the promoter.

In embodiments, the promoter is sensitive to internal stimuli or internal conditions. For example, the promoter may be induced when the bacterial cell culture enters a specific phase of growth, as in case of PCP_2836 promoter (Ma et al., 2018), phoPR promoter (Paul et al., 2004) and P170 promoter (Madsen et al., 1999). Preferably, the promoter may be induced when the bacterial cell culture enters the exponential growth phase, or during the exponential growth phase. Other preferred settings include the use of auto-induction media (for example as described in Studier et al. 2005, supra) and self-inducing systems (for example SILEX, as described in Briand et al. 2016, Scientific Reports, 6:33037).

Promoters which are sensitive to internal conditions are sometimes preferred in industrial settings since these promoters do not require intervention from the user (e.g. the administration of a chemical inducer) to be induced, and, in the present invention thereby induce switching or the irreversible inactivation of the origin replication.

In embodiments, the promoter is sensitive to one or more external stimuli, for example is an inducible promoter. Hence, the promoter used can be selected based on the external stimulus or stimuli which the user wishes to initiate switching (or origin of replication inactivation) with.

In some such embodiments, the promoter is temperature-sensitive, pH-sensitive, light-sensitive, or chemically-sensitive. Promoters with these and other properties, e.g. other inducible properties, are well known and described in the art and appropriate promoters for use in the invention could readily be selected.

For example, other types of gene expression control methods which can be used for the same purpose, include but are not limited to, induction by inducers (IPTG, arabinose, homoserine lactones, anhydrotetracycline etc)(Marschall, Sagmeister and Herwig, 2017)(Lutz and Bujard, 1997)(Cox, Surette and Elowitz, 2007), targeted proteolysis (Cameron and Collins, 2014), different variants of CRISPR/Cas9(Qi et al., 2013) etc. A person skilled in the art would know how to use such other types of gene expression control methods. For example, in the case of chemically induced promoters an appropriate chemical is added to the growth medium where it enters the cells either through diffusion or facilitated transport. Inside the cell it binds to its target transcriptional regulator and this leads to activation of transcription from the target promoters of said transcriptional regulator. If a transcriptional repressor controls the expression from the promoter and said transcriptional repressor is made sensitive to proteolysis then the expression from the promoter can be induced by inducing proteolysis of the said transcriptional regulator. Catalytically inactive CRISPR/Cas9 can be targeted to a promoter by expressing the appropriate guide RNA in the cell, and, depending on the exact configuration this can lead to either activation or repression of the said promoter.

Thus, in embodiments, the promoter is chemically-sensitive and suitable promoters would be readily available in the art. For example, preferred promoters are those wherein the promoter is inducible using isopropyl β-d-1-thiogalactopyranoside (IPTG), arabinose, homoserine lactones, or anhydrotetracycline, e.g. wherein the promoter is a promoter from the lac operon, ara operon, tet operon or lux operon, e.g. wherein the promoter is lac promoter (lac p), araBAD promoter, ppul promoter, tet promoter (Ptet promoter), or luxl promoter.

For example, the bacterial cell can comprise a gene encoding a site-specific recombinase and the expression of said gene can be controlled under a lac operon expression system. The expression of said gene can be induced by IPTG or allolactose. For example, the gene may be provided on a plasmid, wherein the lac promoter and lac operator are provided upstream of the gene and wherein the plasmid further comprises the lac repressor (lac I). Plasmids suitable for control of target genes under this system are known in the art.

In an alternative example the bacterial cell may comprise a gene encoding a site-specific recombinase and the expression of said gene can be controlled under an ara operon expression system. The expression of said gene can be induced by arabinose. For example, the bacterial cell comprises the gene provided on a plasmid, wherein the araBAD promoter is upstream of the gene, and wherein the plasmid also comprises the other genetic elements necessary for gene regulation using the ara operon. The genetic elements of the ara operon necessary for gene regulation are known to the skilled person and are provided above.

In an alternative example the bacterial cell may comprise a gene encoding a site-specific recombinase and the expression of said gene can be controlled under a lux operon expression system. The expression of said gene can be induced by AHL. For example, the bacterial cell comprises the gene provided on a plasmid, wherein the luxl promoter is upstream of the gene, and wherein the plasmid also comprises the luxR gene, preferably wherein the luxR gene is constitutively expressed.

In an alternative example the bacterial cell may comprise a gene encoding a site-specific recombinase and the expression of said gene can be controlled under a Tet-Off or Tet-On expression system. The expression of said gene can be induced by tetracycline (or anhydrotetracycline or doxycycline). For example, the bacterial cell comprises (i) the gene provided on a plasmid, wherein a tetracycline- (or anhydrotetracycline- or doxycycline-) dependent promoter is upstream of the gene, and (ii) a tTA expression plasmid or an rtTA expression plasmid. Plasmids suitable for control of target genes under this system are known in the art.

In embodiments, expression of the gene or the promoter may be induced or controlled by targeted proteolysis.

In embodiments, expression of the gene or the promoter is induced or controlled by CRISPR interference (CRISPRi).

In preferred embodiments, the promoter is temperature-sensitive.

Any appropriate temperature-sensitive promoter can be used, of which there are many examples in the art.

A temperature-sensitive promoter used in the present invention can selected to be activated (and/or at its highest activity level) at any suitable temperature. Preferably, the promoter may be a temperature-sensitive promoter which is activated (and/or at its highest activity level) at between 20 to 25° C., between 25 to 30° C., between 30 to 35° C., between 35 to 40° C., between 40 to 45° C., between 45 to 50° C., between 50 to 55° C., or between 55 to 60° C., or above 60° C. Preferably, the promoter may be activated (and/or at its highest activity level) at about 37° C.

Hence, in embodiments, the temperature-sensitive promoter used in the present invention can be induced by changing (i.e. raising or lowering) the temperature of the bacterial cell (or bacterial cell culture) to the temperature at (or temperature range within) which the temperature-sensitive promoter is activated (and/or at its highest activity level). Hence, in order to induce switching, the temperature may be changed to between 20 to 25° C., between 25 to 30° C., between 30 to 35° C., between 35 to 40° C., between 40 to 45° C., between 45 to 50° C., between 50 to 55° C., or between 55 to 60° C., or above 60° C.

The temperature-sensitive promoter used may be activated at higher temperatures, in which case the temperature of the bacterial cell (or bacterial cell culture) may need to initially be lower (e.g. 30° C. or lower than 30° C.) and then raised (e.g. to 37° C., or higher than 37° C.) in order to induce activation and hence switching (or origin inactivation). Conversely, the temperature-sensitive promoter used may be activated at lower temperatures, in which case the temperature of the bacterial cell (or bacterial cell culture) may need to initially be higher (e.g. 37° C., or higher than 37° C.) and then lowered (e.g. to 30° C., or lower than 30° C.) in order to induce switching (or origin inactivation). Preferably, the promoter may be activated (and/or at its highest activity level) at or about 37° C. (or higher than 37° C.). Thus, conveniently the temperature of the bacterial cell (or bacterial cell culture) may initially be lower, e.g. at or about 30° C., e.g. between 25° C. and 33° C., and then raised (e.g. to at or about 37° C., e.g. between 36° C. and 39° C., or higher than 37° C.) in order to induce switching (or origin inactivation).

A preferred temperature-sensitive promoter for use in the present invention is a promoter which is controllable or regulated by a temperature-sensitive agent (or by a temperature-sensitive repressor, or by a temperature-sensitive activator), for example the phage lambda c1857 repressor. In embodiments, the temperature-sensitive promoter is phage lambda P R or P L promoter (or a modified version of P R or P L promoter), controlled by the lambda c1857 repressor, which are activated (and/or have their highest activity level) at or about 37° C.

(Jechlinger et al., 1999). Thus, use of this promoter conveniently allows induction of switching (or origin inactivation) by changing the temperature of the bacterial cell (or bacterial cell culture) from at or about 30° C. (e.g. between 25° C. and 33° C.), to at or about 37° C. (e.g. between 36° C. and 39° C., or higher than 37° C.).

In embodiments, functional elements, e.g. one or more or all of the functional elements (i.e. genetic elements) used in (or necessary for) the irreversible inactivation of the origin of replication are located on (or integrated in) the genome, for example on a chromosome (i.e. a bacterial chromosome). For example, in embodiments the genetic element(s) (or gene(s)) encoding the site-specific recombinase(s) is located on (or integrated in) a genome, for example on a chromosome (i.e. a bacterial chromosome). Alternatively, the entire recombinase expression system (or switcher system) may be located on (or integrated in) the genome. For example, the genetic elements encoding the site-specific recombinase(s), and the genetic elements encoding the recombinase regulator components, may be located on (or integrated in) the genome, for example on a chromosome. The recombinase regulator components may include a temperature-sensitive promoter as defined herein (for example a P R or P L promoter or a modified version thereof), and optionally where appropriate a gene encoding an agent which facilitates that sensitivity as defined herein, for example a gene encoding a temperature-sensitive repressor, for example a gene encoding the c1857 repressor.

In embodiments, functional elements, e.g. one or more or all of the functional elements (i.e. genetic elements) used in (or necessary for) the irreversible inactivation of the origin of replication are located on (or integrated in) one or more episomes or plasmids, or on some other appropriate extra-chromosomal element within the bacterial cell. For example, in embodiments the genetic element(s) (or gene(s)) encoding the site-specific recombinase(s) is located on (or integrated in) one or more episomes or plasmids, or on one or more other appropriate extra-chromosomal elements within the bacterial cell. Alternatively, the entire recombinase expression system (or switcher system) may be located on (or integrated in) one or more episomes or plasmids (or other appropriate elements). For example, the genetic element(s) (or gene(s)) encoding the site-specific recombinase(s), and the genetic elements (or genes) encoding the recombinase regulator components, may be located on (or integrated in) one or more episomes or plasmids (or other appropriate elements). The recombinase regulator components may include a temperature-sensitive promoter as defined herein (for example a P_{R} or P_{L} promoter or a modified version thereof), and optionally where appropriate a genetic element (or gene) encoding an agent which facilitates that sensitivity as defined herein, for example a gene encoding a temperature-sensitive repressor, for example a gene encoding the c1857 repressor.

Alternatively, the genetic element(s) (or gene(s)) encoding the site-specific recombinase(s) may be located on the genome, while the genetic elements encoding the associated recombinase regulator components may be located on one or more episomes or plasmids; or vice versa.

In embodiments, one or more of the functional elements (i.e. genetic elements) used in (or necessary for) the irreversible inactivation of the origin of replication are codon optimized. For example, the genetic element(s) (or gene(s)) encoding the site-specific recombinase(s) may be codon optimized. Alternatively or in addition, the genetic element (or gene) encoding the temperature-sensitive agent which facilitates the sensitivity of the temperature-sensitive promoter (e.g. encoding a temperature-sensitive repressor, for example encoding c1857), is codon-optimized, where such temperature-sensitive promoter system is used.

In another aspect, the invention provides a modified bacterial cell, wherein the modified bacterial cell lacks a functional origin of replication sequence. Thus, in preferred embodiments such bacterial cells are genetically modified. In other words, the genome or chromosomes of such bacterial cells are modified such that they lack a functional origin of replication. Methods of producing such bacterial cells are described elsewhere herein in connection with the methods of the invention which involve a step of irreversibly inactivating an origin of replication in a bacterial cell.

Thus, a yet further aspect provides a modified bacterial cell, wherein the modified bacterial cell lacks a functional origin of replication sequence, wherein said modified bacterial cell is obtained, produced or obtainable by a method comprising a step of irreversibly inactivating an origin of replication in the bacterial cell. Appropriate ways of carrying out such irreversible inactivation are described elsewhere herein, for example methods comprising genetic modification of the origin of replication, e.g. partial or complete removal of the nucleotide sequence of the origin of replication.

In embodiments, the invention provides a modified bacterial cell, wherein the modified bacterial cell does not contain a (or any) functional origin of replication sequence. Preferably, the modified bacterial cell is not capable of DNA replication (i.e. is not capable of any chromosomal DNA replication).

In embodiments, the invention provides a modified bacterial cell, wherein the modified bacterial cell does not contain a (or any) functional chromosomal origin of replication sequence. Preferably, the modified bacterial cell is not capable of chromosomal DNA replication (i.e. is not capable of any chromosomal DNA replication).

In embodiments, the bacterial cell or modified bacterial cell can produce a product (or is capable of producing a product or is producing a product), e.g a desired product in accordance with the methods of the present invention. Examples of desired products are described elsewhere herein. Thus, a bacterial cell producing a product is a yet further aspect of the invention.

In embodiments, the bacterial cell or modified bacterial cell comprises a gene encoding the product or encoding an enzyme required in order to produce the product. Appropriate genes encoding products or enzymes are also described elsewhere herein, for example the product (and encoding gene) may be an endogenous, homologous, or heterologous product.

In embodiments, the gene is operatively linked to a promoter. Examples of appropriate promoters are described elsewhere herein, and for example can be native or heterologous promoters, or can be constitutive or inducible promoters.

In another aspect, the invention provides a polynucleotide vector comprising an origin of replication flanked by site-specific recombination sites. Appropriate and preferred site-specific recombination sites are described elsewhere herein.

In embodiments, the site-specific recombination sites comprise site-specific recombination sites for recognition by a recombinase, preferably a serine recombinase, preferably wherein the serine recombinase is yO, Bxb1, φC31 or TP901.

In embodiments, the site-specific recombination sites comprise site-specific recombination sites for recognition by a tyrosine recombinase, preferably wherein the tyrosine site-specific recombinase is Cre, Dre, Flp, KD, B2 or B3.

In embodiments, the polynucleotide vector or bacterial cell (modified bacterial cell) of the invention further comprises a marker for selection of the irreversibly (or permanently) inactivatable origin of replication.

A marker for selection is typically a nucleotide sequence encoding a protein that confers selective resistance upon a cell in which it is expressed. The marker for selection is preferably an antibiotic resistance gene. The chloramphenicol resistance gene, the ampicillin resistance gene and the kanamycin resistance gene are preferred selection markers, although many others are described in the art and may be used. The chloramphenicol resistance gene, the ampicillin resistance gene and the kanamycin resistance gene are well-known and well-characterised markers for selection that are routinely used in the field.

Alternatively, the marker for selection may be a gene conferring the ability to use an artificial nitrogen source or gene conferring the ability to use an artificial carbon source. The selection marker enables the selection (or identification) of cells into which the irreversibly (or permanently) inactivatable origin of replication has been integrated/incorporated.

In embodiments, the marker for selection is flanked together with the origin of replication between site-specific recombination sites, so as to act as a positive marker for incorporation of the origin of replication into the chromosome (for example by homologous recombination). Hence, in some embodiments, the irreversibly (or permanently) inactivatable origin of replication comprises, in the 5′ to 3′ direction: (i) a site-specific recombination site, (ii) an origin of replication, (iii) a marker for positive selection, and (iv) a site-specific recombination site; or alternatively the irreversibly (or permanently) inactivatable origin of replication comprises, in the 5′ to 3′ direction: (i) a site-specific recombination site, (ii) a marker for positive selection, (iii) an origin of replication, and (iv) a site-specific recombination site.

The marker for selection can be used for inserting the inactivatable origin of replication into the bacterial chromosome (e.g. by homologous recombination or a different method). However, the marker for selection may be removed later while retaining the inactivatable origin of replication (for example an origin of replication flanked by recombination sites) in the chromosome. Generally, the presence of the marker for selection is not required for downstream applications of the bacterial strains of the invention (e.g. the switcher strains of the invention), e.g. is not required for product production. Thus, it is generally not necessary to maintain the selection marker within the cell once the inactivatable origin of replication has been incorporated into the bacterial chromosome. Alternatively, a strain (e.g. a switcher strain) of the invention may be generated without using a marker for selection, for example using another positive selection tool, or no positive selection tool at all.

In embodiments, the irreversibly (or permanently) inactivatable origin of replication may comprise a (or a further) marker for selection, wherein the marker is arranged such that only the bacterial cells of the invention that have switched (i.e. have had their origin of replication irreversibly (or permanently) inactivated) can express the marker. For example, the marker for selection may be arranged such that it is interrupted by the site-specific recombination sites flanking the origin of replication, such that the full sequence of the marker for selection is only assembled contiguously through removal (or deletion or elimination or excision) of the origin of replication. Alternatively, the marker for selection and its promoter may be arranged such that the marker for selection and its promoter are interrupted by the site-specific recombination sites flanking the origin of replication; in this way, the marker for selection and its promoter are only operatively linked (and hence expression of the marker for selection is only possible) once switching (i.e. irreversible removal of the origin of replication) has taken place.

In embodiments of the irreversibly (or permanently) inactivatable origin of replication, the marker for positive selection is flanked by site-specific recombination sites which are recognisable by a site-specific recombinase enzyme, excluding the origin of replication (i.e. two site-specific recombination sites may be present, one positioned 5′ to the marker for positive selection and one positioned 3′ to the marker for positive selection).

In some embodiments, it is desirable to include positive selection only for cells that have performed the origin of replication elimination from the genome. If the origin of replication elimination switches on the expression of the gene necessary for survival (for example an antibiotic resistance gene or a gene necessary for metabolism of an essential nutrient), only the cells that have switched (or have an inactivated origin of replication) survive. The use of a positive selection step only for cells that have eliminated the origin of replication provides a mechanism to reduce or prevent the survival of rare mutants that arise and have lost their ability to stop growth in response to appropriate induction conditions, e.g. temperature shift, for example, where the origin of replication is no longer removable or is no longer irreversibly inactivatable in accordance with the present invention. Such mutants are also referred to herein as “cheaters” and will potentially continue to grow and multiply under the induction conditions, e.g. 37° C., and may become a dominant population in culture. In some embodiments, this positive selection step can be used to avoid this potential problem.

Another way to avoid this potential problem is through the use of multiple (e.g. 2 or more) site-specific recombinases and their respective site-specific recombination sites as described elsewhere herein.

The bacterial cells of the invention as described above can readily and conveniently be used in the production methods of the present invention, for example cells which are capable of producing the product (desired product). Indeed, the use of such cells is preferred and preferred methods comprise a step of providing such bacterial cells of the invention that are capable of producing said product.

It can also be seen from the above discussion that there are many methods available for the step of irreversibly inactivating an origin of replication in a bacterial cell, and any of these, or indeed any appropriate alternative methods, can be used for this step in the production methods of the invention. Preferred methods are also described above and include the use of methods in which the origin of replication is flanked by recombination sites, e.g. site-specific recombination sites, as described above.

In the methods of (or uses in) producing product (product production methods) as described herein, any further steps required for product production can be included. For example, the bacterial cells producing the product may be cultured, grown or otherwise propagated or produced, by appropriate methods well known in the art in order for product, e.g. appropriate or sufficient amounts of product, to be produced, or for the population of bacterial cells to be grown or expanded. Thus, the methods may further comprise steps of culturing, growing or otherwise propagating the bacterial cells. The methods may also further comprise the steps of collecting, isolating, purifying or otherwise obtaining, the product from the bacterial cells, e.g. from the culture medium (or supernatant) or from the intracellular environment of the bacterial cell. Again, methods for carrying out such steps would be well known in the art.

Isolated or purified bacterial cells (or populations of bacterial cells) are also provided. In some embodiments such bacterial cells will not be, or will not correspond to, naturally occurring bacterial strains. Some embodiments will involve the further steps of culturing or propagating or producing such bacterial strains and optionally formulating said cultured or propagated or produced strains into a composition comprising said strain, e.g. a stable formulation for product production, e.g. for industrial or commercial product production, or into a pharmaceutical composition. Alternatively, such bacterial cells could be stored for future uses, for example through lyophilisation or freezing.

In another aspect, the invention provides the use of a bacterial cell, modified bacterial cell, or polynucleotide vector or method of the invention for producing a product, e.g. a desired product. Exemplary products are described elsewhere herein.

As the bacterial cells (modified bacterial cells) of the invention can be engineered to produce products of interest, including therapeutically effective and useful products, and in useful or significant quantities, such bacteria have clear therapeutic uses, for example can be administered to subjects or patients, in particular human patients, where administration of that product is therapeutically effective or useful. Use of bacteria as living medicines, e.g. probiotics, is accepted in the art and the use of the bacterial cells of the invention in any therapies where the use of such living medicines is suitable or appropriate are up contemplated by the present invention.

Thus, in another aspect, the invention provides the bacterial cell of the invention, or the modified bacterial cell of the invention, for use in therapy.

In another aspect, the invention provides the bacterial cell of the invention, or the modified bacterial cell of the invention, for use in the treatment or prevention of a disease or pathology. In preferred embodiments, the disease or pathology is cancer, metabolic disease or an immunological disorder.

In another aspect, the invention provides the use of a bacterial cell of the invention, or the modified bacterial cell of the invention, in the manufacture of a medicament for the treatment or prevention of a disease or pathology. In preferred embodiments, the disease or pathology is cancer, metabolic disease or an immunological disorder.

In another aspect, the invention provides a method of treatment or prevention of a disease comprising administering an effective amount of the bacterial cell of the invention, or the modified bacterial cell of the invention, to a subject, preferably wherein the disease or pathology is cancer, a metabolic disease, or an immunological disorder.

In embodiments, the disease or pathology is a metabolic disease (or metabolic disorder).

Preferably, the metabolic disease (or metabolic disorder) is an acid-base imbalance, a metabolic brain disease, a calcium metabolism disorder, a DNA repair-deficiency disorder, a glucose metabolism disorder, hyperlactatemia, an iron metabolism disorder, a lipid metabolism disorder, a malabsorption syndrome, metabolic syndrome, an inborn error of metabolism, a mitochondrial disease, a phosphorus metabolism disorder, a porphyria, a proteostasis deficiency, a metabolic skin disease, wasting syndrome (or cachexia), or a water-electrolyte imbalance.

In embodiments, the disease or pathology is an immunological disorder (or an immunological disease). Preferably, the immunological disorder (or immunological disease) is allergy, asthma, an autoimmune disease (for example lupus, scleroderma, hemolytic anemia, vasculitis, type 1 diabetes, Graves' disease, rheumatoid arthritis, multiple sclerosis, Goodpasture syndrome, Pernicious anemia, myopathy or Lyme disease), an autoinflammatory syndrome or an immunological deficiency syndrome.

The term “subject” (or “patient”) as used herein includes any mammal, for example humans and any livestock, domestic or laboratory animal. Specific examples include mice, rats, pigs, cats, dogs, horses, sheep, rabbits, cows and monkey (or other primate). Preferably, however, the patient is a human subject.

In embodiments relating to therapeutic methods and uses described herein, appropriate subjects are those having, suspected of having, or at risk of having (or susceptible to) the disease to be treated.

The administration of the bacterial strains in said therapeutic methods and uses of the invention is carried out in pharmaceutically, therapeutically, or physiologically effective amounts, to subjects in need of treatment. Thus, said methods and uses may involve the additional step of identifying a subject in need of treatment.

Treatment of disease in accordance with the present invention (for example treatment of pre-existing disease) includes cure of said disease or conditions, or any reduction or alleviation of disease (e.g. reduction in disease severity) or symptoms of disease.

The methods and uses of the prevent invention are suitable for prevention of diseases as well as active treatment of diseases (for example treatment of pre-existing disease). Thus, prophylactic treatment is also encompassed by the invention. For this reason in the methods and uses of the present invention, treatment also includes prophylaxis or prevention where appropriate.

In embodiments, the bacterial cell is a Gram-negative bacterial cell. Alternatively, the bacterial cell is a Gram-positive bacterial cell.

In embodiments, the bacterial cell is Escherichia sp., Bacillus sp., Lactococcus sp., 5 Streptococcus sp., Lactobacillus sp., Corynebacterium sp., Streptomyces sp., Pseudomonas sp., Clostridium sp., Xanthomonas sp, or Enterobacteriaceae.

In embodiments, the bacterial cell is Escherichia sp., more preferably Escherichia coli, more preferably Escherichia coli MG1655.

In embodiments, the bacterial cell is Bacillus sp., preferably Bacillus subtilis.

As used throughout the application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms. Methods comprising certain steps also include, where appropriate, methods consisting of these steps.

The term “increase” or “enhance” (or equivalent terms) as described herein includes any measurable increase or elevation when compared with an appropriate control. Appropriate controls would readily be identified by a person skilled in the art and appropriate examples are described herein. Preferably the increase will be significant, for example clinically or statistically significant, for example with a probability value of ≤0.05, when compared to an appropriate control level or value.

The term “decrease” or “reduce” (or equivalent terms) as described herein includes any measurable decrease or reduction when compared with an appropriate control. Appropriate controls would readily be identified by a person skilled in the art and appropriate examples are described herein. Preferably the decrease will be significant, for example clinically or statistically significant, for example with a probability value of ≤0.05, when compared to an appropriate control level or value.

Methods of determining the statistical significance of differences between test groups of subjects or differences in levels or values of a particular parameter are well known and documented in the art. For example herein a decrease or increase is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of 0.05.

The invention will now be further described in the following non-limiting Examples with reference to the following figures.

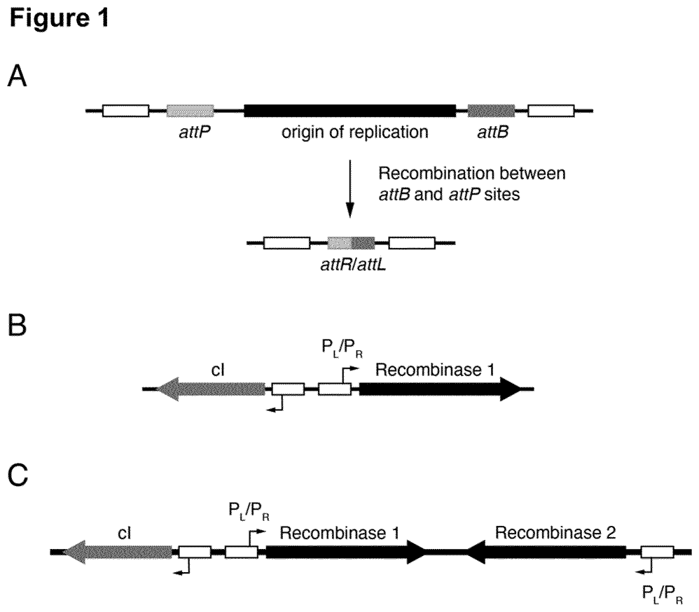

FIG. 1. Genetic basis of the switcher strain. (A) Serine recombinase recognition sites attB and attP are integrated either side of oriC in the bacterial chromosome in an orientation such that recombination leads to excision of oriC from the bacterial chromosome. (B) A serine recombinase is placed under the control of phage lambda P_{R} or P_{L} promoter that is repressed by constitutively-expressed temperature-sensitive phage lambda cl repressor. (C) Dual recombinase setup, where both of the recombinases are under the control of lambda cl repressor.

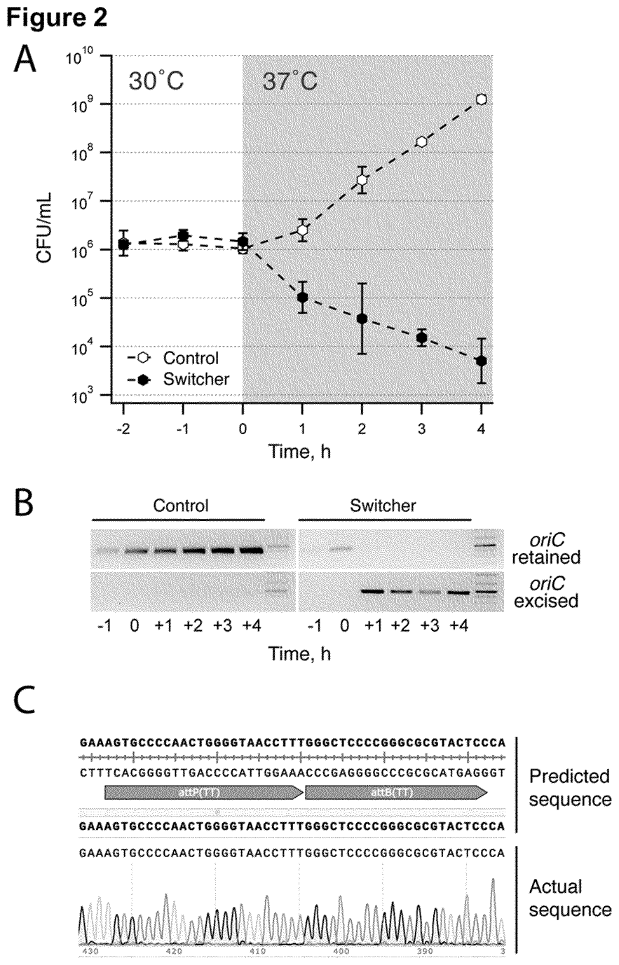

FIG. 2. Temperature-induced expression of φC31 integrase leads to excision of oriC in the switcher cells. Control (bAJ83, empty symbols) and the switcher (bAJ84, filled symbols) cultures were diluted from overnight culture and grown in a shaking flask at 30° C. (white plot area) for two hours. At timepoint zero the temperature was changed to 37° C. (grey plot area). (A) The number of colony forming units was measured before and after the temperature change. The geometric mean of three independent experiments is plotted, error bars indicate standard deviation. (B) The oriC excision from chromosomal DNA was detected by PCR using primers unique to either the unrecombined (oriC retained) or recombined (oriC excised) DNA sequence, and visualised by agarose gel electrophoresis. (C) The DNA sequence of the junction formed between attB and attP sites after the oriC excision by φC31 integrase.

FIG. 3. Final cell density can be tuned by the timing of the switch. Various dilutions from the overnight culture of the control strain (bAJ83, empty symbols) and switcher strain (bAJ84, filled symbols) were inoculated at 30° C. (white plot area) on a microplate incubator. At timepoint zero hours the temperature was changed to 37° C. (grey plot area) to induce the switching. The switcher cells stopped growing at different levels of biomass produced (optical density at 600 nm), depending on the density at the time of the switch. The mean of six biological replicates from two independent experiments is plotted, shading indicates standard deviation.

FIG. 4. The assessment of protein production capability of switcher strain compared with control strain. Control (bAJ85, empty symbols) and switcher (bAJ86, filled symbols) cultures were pre-grown at 30° C. (white plot area) on a microplate incubator, seeded from overnight cultures at different dilutions (2400× in A, 1600× in B). At timepoint zero hours the temperature was changed to 37° C. (grey plot area) to induce oriC excision in the switcher strain. The production of fluorescent mRFP1 protein was induced by adding HSL at timepoint 0. (A, B) The change in OD at 600 nm (left axis) and increase in fluorescence (excitation: 584 nm, emission: 607 nm) of mRFP1 protein (right axis) was monitored. (C, D) Fluorescence over optical density (biomass) ratio of control (empty) and the switcher (filled) culture was calculated based on values of panels A and B and plotted (2400× in C, 1600× in D). The mean of three biological replicates is plotted, shading indicates standard deviation.

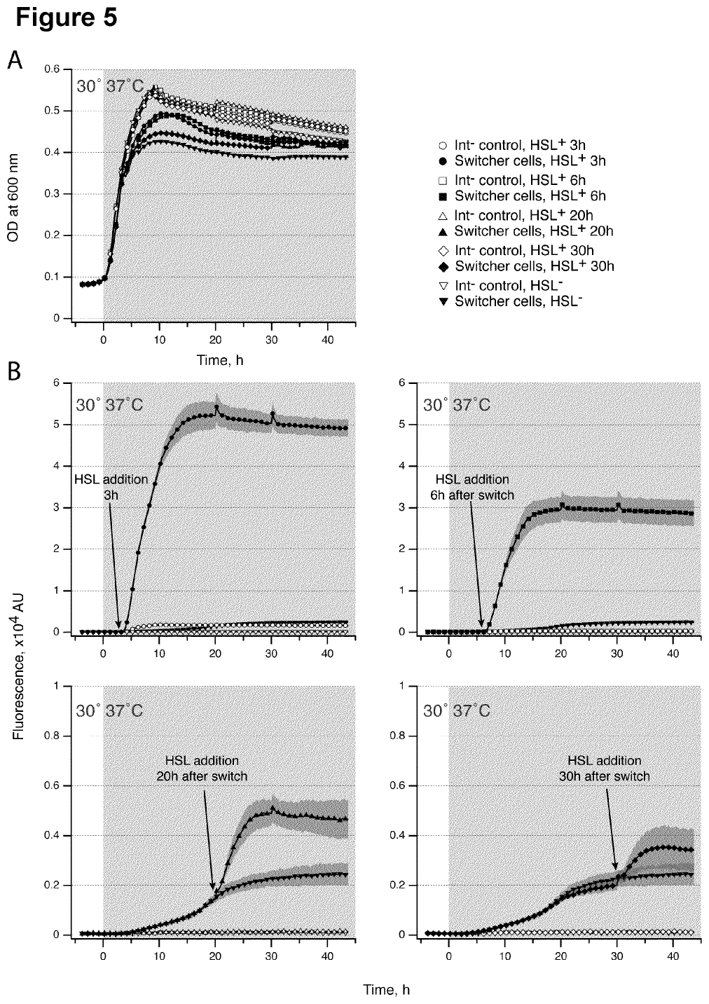

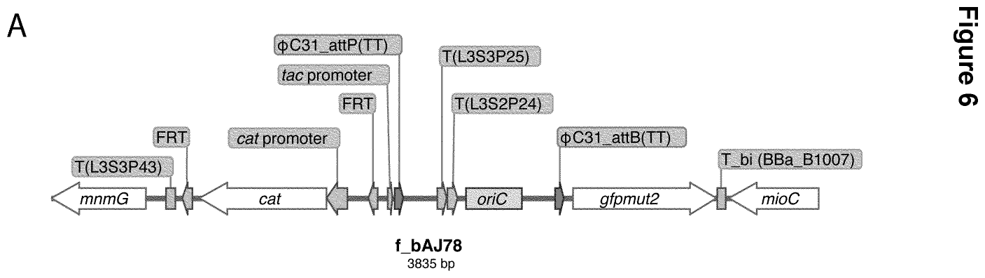

FIG. 5. The protein production capability of the switcher strain remains active over a long period of time. Control (bAJ85, empty symbols) and switcher (bAJ86, filled symbols) cultures were pre-grown at 30° C. (white plot area) on a microplate incubator. At timepoint zero hours the temperature was changed to 37° C. (grey plot area) to induce oriC excision in the switcher strain. The production of fluorescent mRFP1 reporter protein was induced by adding HSL to both control and switcher cultures at indicated timepoints. The change in optical density (A) and fluorescence (B, excitation: 584 nm, emission: 607 nm) was monitored. The mean of three biological replicates is plotted, shading (panel B) indicates standard deviation.

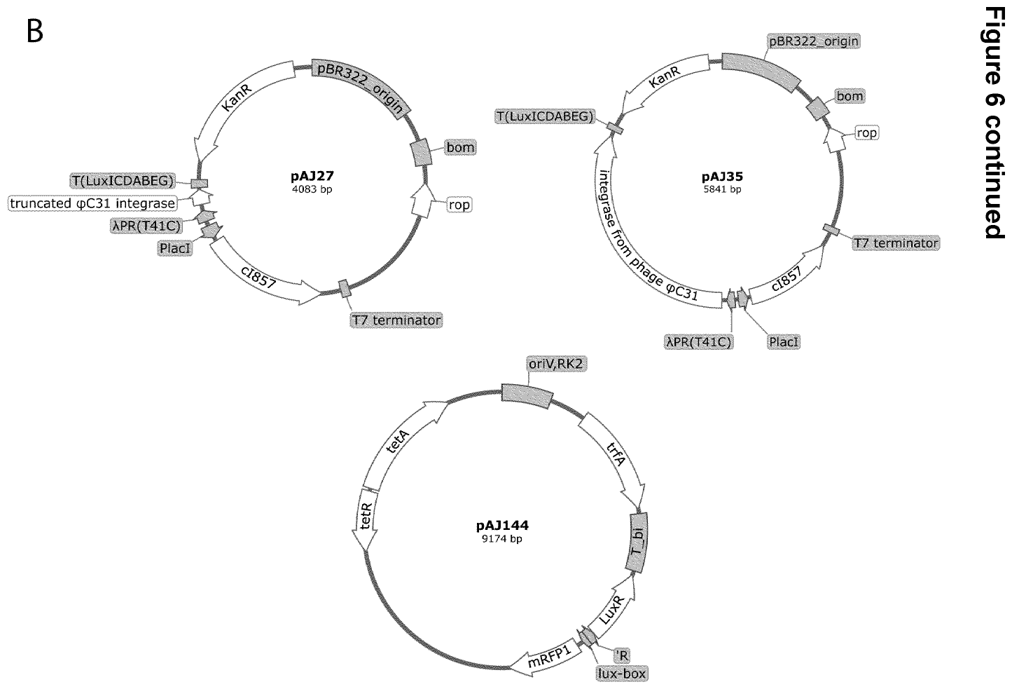

FIG. 6. Schematic representation of DNA constructs and plasmids used. A. Linear DNA fragment incorporated into genome of switcher strain. The region between mnmG and mioC genes in the genome was replaced with f_bAJ78 DNA fragment. T— transcription terminator, T_bi—bidirectional transcription terminator, FRT— flippase recognition target site, cat—chloramphenicol resistance gene, gfpmut2— GFP reporter gene. B. Maps of plasmids used in this study. λP_{R}(T41C)— phage λP_{R}(T41C) promoter, bom, rop, trfA, oriV, RK2— elements of plasmid DNA origin of replication, pBR322_origin—plasmid origin of replication, tetA, tetR— tetracyclin resistance genes, KanR— kanamycin resistance gene, lux-box—promoter regulated by LuxR.

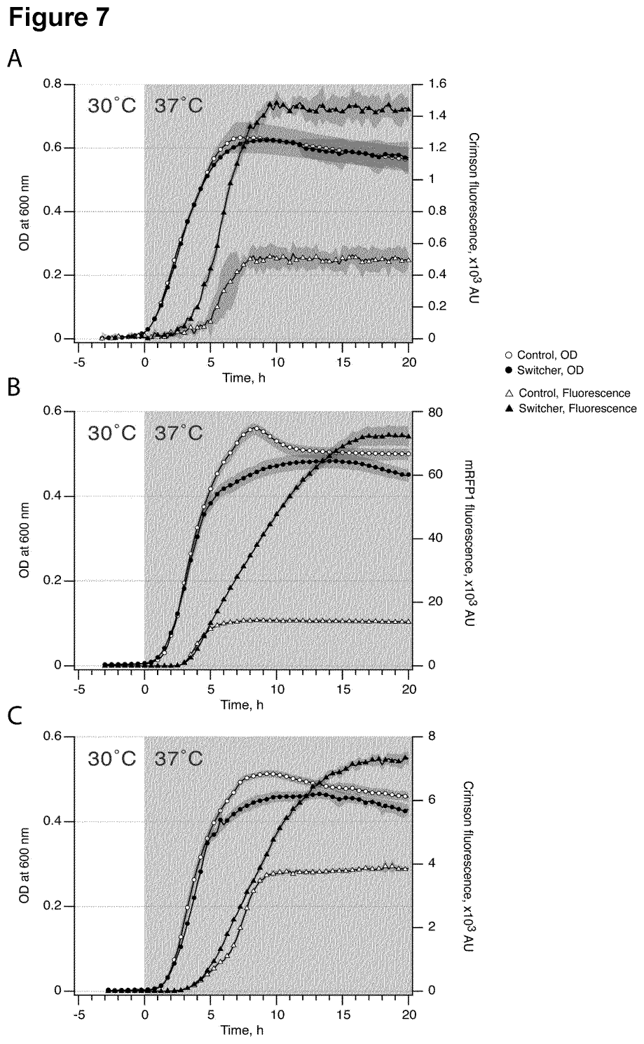

FIG. 7A. The assessment of protein production capability of switcher strain compared with control strain. Control (bAJ139, empty symbols) and switcher (bAJ140, filled symbols) cultures were pre-grown at 30° C. (white plot area) on a microplate incubator, seeded from overnight cultures at 2000× dilution. At timepoint zero hours the temperature was changed to 37° C. (grey plot area) to induce oriC excision in the switcher strain. The production of fluorescent E2-Crimson protein was induced by adding IPTG at timepoint 0. The change in OD at 600 nm (left axis) and increase in fluorescence (excitation: 610 nm, emission: 650 nm) of E2-Crimson protein (right axis) was monitored. The mean of three biological replicates is plotted, shading indicates standard deviation.

FIG. 7B. The assessment of protein production capability of genomic switcher strain compared with control strain. Control (bAJ85, empty symbols) and switcher (bAJ178, filled symbols) cultures were pre-grown at 30° C. (white plot area) on a microplate incubator, seeded from overnight cultures at 8000× dilution. At timepoint zero hours the temperature was changed to 37° C. (grey plot area) to induce oriC excision in genomic switcher strain. The production of fluorescent mRFP1 was induced by adding HSL at timepoint 0. The change in OD at 600 nm (left axis) and increase in fluorescence (excitation: 584 nm, emission: 607 nm) of mRFP1 protein (right axis) was monitored. The mean of three biological replicates is plotted, shading indicates standard deviation.

FIG. 7C. The assessment of protein production capability of genomic switcher strain compared with control strain. Control (bAJ43, empty symbols) and switcher (bAJ176, filled symbols) cultures were pre-grown at 30° C. (white plot area) on a microplate incubator, seeded from overnight cultures at 16000× dilution. At timepoint zero hours the temperature was changed to 37° C. (grey plot area) to induce oriC excision in the all-genomic switcher strain. The production of fluorescent E2-Crimson protein was induced by adding IPTG at timepoint 0. The change in OD at 600 nm (left axis) and increase in fluorescence (excitation: 610 nm, emission: 650 nm) of E2-Crimson protein (right axis) was monitored. The mean of three biological replicates is plotted, shading indicates standard deviation.

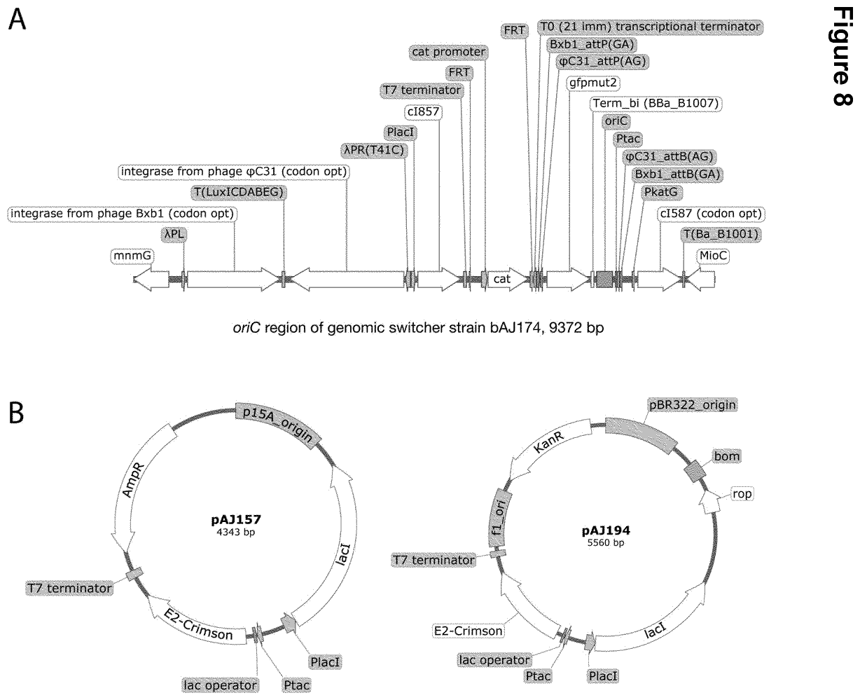

FIG. 8. Schematic representation of DNA constructs and plasmids used. A. Linear DNA fragment incorporated into genome of genomic switcher strain bAJ174. The region between mnmG and mioC genes in the genome was replaced with bAJ174 DNA fragment. T— transcription terminator, T_bi—bidirectional transcription terminator, FRT— flippase recognition target site, cat—chloramphenicol resistance gene, gfpmut2— GFP reporter gene. B. Maps of plasmids used in this study. bom, rop,— elements of plasmid DNA origin of replication, p15A_orign, pBR322_origin—plasmid origins of replication, lac operator—lacl binding sequence, AmpR— ampicillin resistance gene, KanR— kanamycin resistance gene.

### EXAMPLE 1

### Materials and Methods

Bacterial Strains, Plasmids, and Growth Medium

Strains and plasmids are listed in Table 1. E. coli DH5a was used for plasmid cloning and propagation. Genomic alterations and plasmid-based switcher experiments were performed in E. coli MG1655. E. coli was grown in lysogeny broth (LB) supplemented with the appropriate amount of antibiotics (100 μg/ml ampicillin, 25 μg/ml chloramphenicol, and 25 μg/ml kanamycin, 10 μg/ml tetracycline) when necessary for the selection of strains and maintenance of the plasmids.

DNA manipulations

Short oligonucleotide sequences for cloning and sequencing were ordered from Metabion International AG.

E. coli MG1655 genomic in situ engineering was performed using recombineering. The Lambda Red recombination system was expressed from pKD46 plasmid (Datsenko and Wanner, 2000). The oriC fragment with flanking attB and attP sites (Table 2) was ordered as synthetic DNA from Twist Bioscience. Flanking 400 bp long homologous regions were amplified from the genomic DNA of E. coli MG1655. Chloramphenicol resistance marker with adjacent FRT sites was amplified from pKD3. Recombineering fragment f_bAJ78 (final sequence listed in Table 3, FIG. 6A) was assembled using overlap extension PCR, gel purified, and transformed into E. coli cells by electroporation.

For construction of genomic switcher strain, codon-optimised sequences of φC31 integrase, Bxb1 integrase with λP_{L} promoter, c1857 repressor with PkatG promoter, attB and attP sites, and cat selection marker gene were ordered as synthetic DNA fragments from Twist Bioscience (Tables 2, 5). OriC and homologous regions required for recombineering were amplified from E. coli MG1655 genomic DNA by PCR. Fragments were assembled by overlap extension PCR, gel purified, and used for recombineering (Datsenko and Wanner, 2000) (final sequence of bAJ174 integrated into genome is listed in Table 3, FIG. 8A).

Plasmid pAJ35 was used for the expression of φC31 integrase in the switcher strain. In pAJ35 the φC31 integrase (Merrick, Zhao and Rosser, 2018) was placed under the control of mutated λP_{R}(T41C) promoter (Table 2; (Jechlinger et al., 1999)) whose activity was controlled by temperature sensitive mutant of phage A repressor c1587 (Jechlinger et al., 1999) expressed from a constitutive promotor in pET24 backbone (Table 4, FIG. 6B). In control strains plasmid pAJ27 containing all the same elements as pAJ35, except φC31 integrase ORF was replaced with small ORF translating MDTYAGAYDRQSRERENSSAASPATQRSA, was used as negative control (Table 4, FIG. 6B).

For the determination of protein synthesis activity, a red fluorescent protein mRFP1 (Campbell et al., 2002) (Campbell et al., 2002) was placed under the control of HSL inducible Lux promoter in plasmid pAJ144 (Table 4, FIG. 6B).

For the determination of IPTG-induced protein synthesis activity, a fluorescent protein E2-Crimson (Strack et al., 2009) was placed under the control of IPTG inducible Ptac promoter in plasmids with either pBR322 origin of replication (pAJ194) or p15A origin of replication (pAJ157) (Table 4, FIG. 8B).

Validation of oriC Excision

Cultures of switcher and control strains were grown overnight at 30° C., diluted 2000 times into 20 mL of fresh LB and grown at 30° C. for 2 hours. Thereafter the temperature was shifted to 37° C. Samples were taken hourly to count colony forming units per volume of culture (CFU/mL) on LB-agar plates, and to analyse the excision of oriC by PCR. The amount of template was normalized by OD. Specific primer pairs were used to detect either excised (oAJ297 and oAJ32) or unexcised (oAJ91 and oAJ32) version of oriC. The DNA sequence of excised versions of oriC was verified by sequencing.

Measurement of Growth and Fluorescence

Cultures of switcher and control strains were grown overnight at 30° C., diluted as indicated in the figure legends, continued to grow on a 96-well plate at 30° C. for 3 (for genomic switcher) or 4 hours until the temperature was shifted to 37° C. (timepoint 0 h). Where appropriate, the induction of mRFP1 synthesis was induced by adding 12 μM HSL (final concentration) either immediately prior temperature shift or at indicated timepoints. The growth curves and mRFP1 related fluorescence (reflecting protein level) were determined using BioTek Synergy MX plate reader. The optical density at 600 nm and fluorescence intensity (excitation at 584/13.5 nm and emission at 607/13.5 nm) were measured in a culture volume of 100 μL. Synthesis of E2-Crimson was induced by adding 0.5 mM IPTG (final concentration) immediately prior temperature shift and fluorescence intensity (excitation at 610/20 nm and emission at 650/20 nm) were measured in a culture volume of 100 μL.

TABLE 1
Strains and plasmids

| | | Source/ |
|---|---|---|
| | Description | Reference |
| Strain | | |
| DH5α | F⁻ endA1 glnV44 thi-1 recA1 relA1 gyrA96 | laboratory |
| | deoR nupG purB20 ϕ80dlacZΔM15 | stock |
| | Δ(lacZYA-argF)U169, hsdR17(r_{K}⁻m_{K}⁺), λ⁻ | |
| MG1655 | K-12 F⁻λ⁻ ilvG⁻ rfb-50 rph-1 | CGSC |
| bAJ43 | MG1655/pAJ194 | This study |
| bAJ78 | MG1655 ΔoriC::cat-attP-oriC-attB, Cm^{R} | This study |
| bAJ83 | bAJ78/pAJ27, Cm^{R} Kan^{R} | This study |
| bAJ84 | bAJ78/pAJ35, Cm^{R} Kan^{R} | This study |
| bAJ85 | bAJ78/pAJ27/pAJ144, Cm^{R} Kan^{R} Tet^{R} | This study |
| bAJ86 | bAJ78/pAJ35/pAJ144, Cm^{R} Kan^{R} Tet^{R} | This study |
| bAJ139 | bAJ78/pAJ27/pAJ157, Cm^{R} Kan^{R} Amp^{R} | This study |
| bAJ140 | bAJ78/pAJ35/pAJ157, Cm^{R} Kan^{R} Amp^{R} | This study |
| bAJ174 | MG1655 ΔoriC::λP_{L}-intBxB-λP_{R}(T41C)- | This study |
| | intφC31-Placl-cl857-cat-attP-oriC-attB- | |
| | PkatG-cl857(opt), Cm^{R} | |
| bAJ176 | bAJ174/pAJ194, Cm^{R} Kan^{R} | This study |
| bAJ178 | bAJ174/pAJ144, Cm^{R} Tet^{R} | This study |
| Plasmid | | |
| pAJ27 | Kan^{R}, pBR322_Placl-cl857, integrase | This study |
| | negative control | |
| pAJ35 | Kan^{R}, pBR322_Placl-cl857_λP_{R}(T41C)- | This study |
| | intφC31 | |
| pAJ144 | Tet^{R}, pJB_LuxR-6His-mRFP | This study |
| pAJ157 | Amp^{R}, p15A_Ptac-Crimson | This study |
| pAJ194 | Kan^{R}, pBR322_Ptac-Crimson | This study |
| pKD46 | Amp^{R}, λ-Red helper plasmid (recombinase) | Datsenko & |
| | | Wanner, 2000 |

^{a} Cm^{R}, chloramphenicol resistance; Kan^{R}, kanamycin resistance; Amp^{R}, ampicillin resistance; Tet^{R}, tetracycline resistance

TABLE 2
The nucleotide sequence of functional elements and
sequences of oligonucleotides

| | |
|---|---|
| IntφC31attB(TT) | GTGCGGGTGCCAGGGCGTGCCCTTGGGCTCCCCGGGCGCGTACTCC |
| | (SEQ ID NO: 1) |
| IntφC31attP(TT) | AGTGCCCCAACTGGGGTAACCTTTGAGTTCTCTCAGTTGGGGGCGT |
| | (SEQ ID NO: 2) |
| IntφC31attB(AG) | GTGCGGGTGCCAGGGCGTGCCCAGGGGCTCCCCGGGCGCGTACTCC |
| | (SEQ ID NO: 3) |
| IntφC31attP(AG) | AGTGCCCCAACTGGGGTAACCTAGGAGTTCTCTCAGTTGGGGGCGT |
| | (SEQ ID NO: 4) |
| IntBxb attB(GA) | CGGCCGGCTTGTCGACGACGGCGGACTCCGTCGTCAGGATCATCCG |
| | GGC (SEQ ID NO: 5) |
| IntBxb attP(GA) | GTCGTGGTTTGTCTGGTCAACCACCGCGGACTCAGTGGTGTACGGT |
| | ACAAACCCA (SEQ ID NO: 6) |
| λP_{R}(T41C)^{a} | TATCACCGCAAGGGATAAATATCTAACACCGCGCGTGTTGACTATT |
| | TTACCTCTGGCGGTGATAATGGTTGCA (SEQ ID NO: 7) |
| λP_{L} | AACCATCTGCGGTGATAAATTATCTCTGGCGGTGTTGACATAAATA |
| | CCACTGGCGGTGATA (SEQ ID NO: 8) |
| PkatG | TAATCAAAAAAGCTTAATTAAGATCAATTTGATCTACATCTCTTTA |
| | ACCAACAATATGTAAGATCTCAACTATCGCATCCGTGGATTAATTC |
| | AATTATAACTTCTCTCTAACGCTGTGTATCGTAACGGTAACACTGT |
| | AGA (SEQ ID NO: 9) |
| oAJ32 | CTCGATTCTATTAACAAGGGTATCACC (SEQ ID NO: 10) |
| oAJ91 | GTGCGGGTGCCAGGGCGTG (SEQ ID NO: 11) |
| oAJ297 | GGGGAGCCCAAAGGTTACC (SEQ ID NO: 12) |

^{a}T41C mutation is indicated in bold

TABLE 3
Sequences of oriC regions of switcher strains

>f_bAJ78 (SEQ ID NO: 13)
AACGTCCCAACGGTGAGCACGACGGCTTTGGCACGGAACTTCAGTCCCATTTGGGTAACAGCACCGACCACGCGATCGTTTTCGACA
ATAAGATCTTCAACCGCCTGCTGGAAGATCATCAGGTTCGGTTGGTTCTCCAGCGCCGTACGTACCGCCTGACGGTAGAGCACACGA
TCCGCCTGAGCTCGGGTAGCGCGAACCGCCGGTCCTTTGCTTGCGTTTAGTATCCTAAACTGGATACCCGCCTGATCGATCGCTTTC
GCCATCAGACCGCCGAGTGCATCCACTTCTTTTACCAGATGTCCCTTCCCAATACCGCCGATCGCCGGGTTGCAGCTCATCTGCCCC
AGAGTGTCGATATTGTGTGTCAAAAGCAGAGTCTGTTGACCCATACGCGCCGCGGCCATCGCGGCCTCGGTGCCTGCATGACCCCCG
CCAATGATGATGACGTCAAAAGGATCCGGATAAAACATGGTGATTGCCTCGCATAACGCGGTATGAAAATGGATTGAAGCCCGGGCC
GTGGATTCTACTCAACTTTGTCGGCTTGAGAAAGTAGGGAACTGCCAGGCATGGGAGACCAGAAAAGAAAAAACACCCGTTAGGGTG
TTTTTAGTTAGAACGTCTTGAGCGATTGTGTAGGCTGGAGCTGCTTCGAAGTTCCTATACTTTCTAGAGAATAGGAACTTCGGAATA
GGAACTTCATTTAAATGGCGCGCCTTACGCCCCGCCCTGCCACTCATCGCAGTACTGTTGTATTCATTAAGCATCTGCCGACATGGA
AGCCATCACAAACGGCATGATGAACCTGAATCGCCAGCGGCATCAGCACCTTGTCGCCTTGCGTATAATATTTGCCCATGGTGAAAA
CGGGGGCGAAGAAGTTGTCCATATTGGCCACGTTTAAATCAAAACTGGTGAAACTCACCCAGGGATTGGCTGAGACGAAAAACATAT
TCTCAATAAACCCTTTAGGGAAATAGGCCAGGTTTTCACCGTAACACGCCACATCTTGCGAATATATGTGTAGAAACTGCCGGAAAT
CGTCGTGGTATTCACTCCAGAGCGATGAAAACGTTTCAGTTTGCTCATGGAAAACGGTGTAACAAGGGTGAACACTATCCCATATCA
CCAGCTCACCGTCTTTCATTGCCATACGTAATTCCGGATGAGCATTCATCAGGCGGGCAAGAATGTGAATAAAGGCCGGATAAAACT
TGTGCTTATTTTTCTTTACGGTCTTTAAAAAGGCCGTAATATCCAGCTGAACGGTCTGGTTATAGGTACATTGAGCAACTGACTGAA
ATGCCTCAAAATGTTCTTTACGATGCCATTGGGATATATCAACGGTGGTATATCCAGTGATTTTTTTCTCCATTTTAGCTTCCTTAG
CTCCTGAAAATCTCGACAACTCAAAAAATACGCCCGGTAGTGATCTTATTTCATTATGGTGAAAGTTGGAACCTCTTACGTGCCGAT
CAACGTCTCATTTTCGCCAAAAGTTGGCCCAGGGCTTCCCGGTATCAACAGGGACACCAGGATTTATTTATTCTGCGAAGTGATCTT
CCGTCACAGGTAGGCGCGCCGAAGTTCCTATACTTTCTAGAGAATAGGAACTTCGGAATAGGAACTAAGGAGGATATTCATATGCCG
TCAATTGTCTGATTCGTTACCAAAAAGGCTTGACAATTAATCATCGGCTCGTATAATGTGTGGAAAGTGCCCCAACTGGGGTAACCT
TTGAGTTCTCTCAGTTGGGGGCGTTTAATAAACTATGGAAGTATGTACAGTCTTGCAATGTTGAGTGAACAAACTTCCATAATAAAA
TTGCGAGTTTTTATTTCGTTTATTTCAATTAAGGTAACTAAAAAACTCCTTTACGTCGTGGTTTGTCTGGTCAACCACCGCGGTCTC
AGTGGTGTACGGTACAAACCCAATTATTGAAGCGGCTAACGCCGCTTTTTTTGTTTCTGGTCTCCCCTCGGTACCAAATTCCAGAAA
AGACACCCGAAAGGGTGTTTTTTCGTTTTGGTCCGCTATAGCGCTCGAAGGGATCGCTAAGCGCTAGAGCTTAATACTGTGGATAAC
TCTGTCAGGAAGCTTGGATCAACCGGTAGTTATCCAAAGAACAACTGTTGTTCAGTTTTTGAGTTGTGTATAACCCCTCATTCTGAT
CCCAGCTTATACGGTCCAGGATCACCGATCATTCACAGTTAATGATCCTTTCCAGGTTGTTGATCTTAAAAGCCGGATCCTTGTTAT
CCACAGGGCAGTGCGATCCTAATAAGAGATCACAATAGAACAGATCTCTAAATAAATAGATCTTCTTTTTAATACCCAGGATCCCAG
GTCTCGGCCGGCTTGTCGACGACGGCGGTCTCCGTCGTCAGGATCATCCGGGCATGCCAACACAATTAACATCTCAATCAAGGTAAA
TGCTTTTTGCTTTTTTTGCCAATCATCAGATAACTATGGCGGCACGTGCATTAACCACGGTTGTATCCCGTCTAAAGTACTCGTGTG
CGGGTGCCAGGGCGTGCCCTTGGGCTCCCCGGGCGCGTACTCCCACTGCTCTTTAACAATTTATCAGATCCAATAGGAGGAACAATA
TGAAAGGATCCAAAGGTGAAGAATTATTCACTGGAGTTGTCCCAATTCTTGTTGAATTAGATGGTGATGTTAATGGGCACAAATTTT
CTGTCAGTGGAGAGGGTGAAGGTGATGCAACATACGGAAAACTTACCCTTAAATTTATTTGCACTACTGGAAAACTACCTGTTCCAT
GGCCAACACTTGTCACTACTTTCGCGTATGGTCTTCAATGCTTTGCGAGATACCCAGATCATATGAAACAGCATGACTTTTTCAAGA
GTGCCATGCCCGAAGGTTATGTACAGGAAAGAACTATATTTTTCAAAGATGACGGGAACTACAAGACACGTGCTGAAGTCAAGTTTG
AAGGTGATACCCTTGTTAATAGAATCGAGTTAAAAGGTATTGATTTTAAAGAAGATGGAAACATTCTTGGACACAAATTGGAATACA
ACTATAACTCACACAAAGTATACATCATGGCAGACAAACAAAAGGATGGAATCAAAGTTAACTTCAAAATTAGACACAACATTGAAG
ATGGAAGCGTTCAACTAGCAGACCATTATCAACGAAATACTCCAATTGGCGATGGCCCTGTCCTTTTACCAGACAACCATTACCTGT
CCACACAATCTGCCCTTTCGAAAGATCCCAACGAAAAGAGAGACCACATGGTCCTTCTTGAGTTTGTAACAGCTGCTGGGATTACAC
ATGGTATGGATGAATTGTACAAATAACGCAAAAAACCCCGCCCCTGACAGGGGGGGGTTTTTTCGCGATCGCACGATCTGTATACTT
ATTTGAGTAAATTAACCCACGATCCCAGCCATTCTTCTGCCGGATCTTCCGGAATGTCGTGATCAAGAATGTTGATCTTCAGTGTTT
CGCCTGTCTGTTTTGCACCGGAATTTTTGAGTTCTGCCTCGAGTTTATCGATAGCCCCACAAAAGGTGTCATATTCACGACTGCCAA
TACCGATTGCGCCAAAGCGGACTGCAGAAAGATCGGGCTTCTGTTCCTGCAATGCTTCATAGAAAGGAGAAAGGTTGTCCGGAATAT
CTCCGGCACCGTGGGTGGAGCTGATAACCAGCCAGATCCCTGAGGCAGGTAAATCTTCTAACAGCGGACCGTGCAGCGTTTCGGTGG
TAAAACCCGCCTCTTCCAGCTTTTCAGCCAGGTGTTCTGCTACATATTCGGCACCGCCGAGGGTGCTGCCGCTGATAAGAGTGATAT
CTGCCAT
>genomic switcher bAJ174 (SEQ ID NO: 14)
AACGTCCCAACGGTGAGCACGACGGCTTTGGCACGGAACTTCAGTCCCATTTGGGTAACAGCACCGACCACGCGATCGTTTTCGACA
ATAAGATCTTCAACCGCCTGCTGGAAGATCATCAGGTTCGGTTGGTTCTCCAGCGCCGTACGTACCGCCTGACGGTAGAGCACACGA
TCCGCCTGAGCTCGGGTAGCGCGAACCGCCGGTCCTTTGCTTGCGTTTAGTATCCTAAACTGGATACCCGCCTGATCGATCGCTTTC
GCCATCAGACCGCCGAGTGCATCCACTTCTTTTACCAGATGTCCCTTCCCAATACCGCCGATCGCCGGGTTGCAGCTCATCTGCCCC
AGAGTGTCGATATTGTGTGTCAAAAGCAGAGTCTGTTGACCCATACGCGCCGCGGCCATCGCGGCCTCGGTGCCTGCATGACCCCCG
CCAATGATGATGACGTCAAAAGGATCCGGATAAAACATGGTGATTGCCTCGCATAACGCGGTATGAAAATGGATTGAAGCCCGGGCC
GTGGATTCTACTCAACTTTGTCGGCTTGAGAAACGCAAAAAACCCCGCCCCTGACAGGGGGGGGTTTTTTCGCGTGCGGGTGCCAGG
GCGTGCCCTCGAGTTCTCTCAGTTGGGGGCGTGCTATTCGATAGTTGTTAAGGTCGCGGTCTGACGCTCAGTGGAACAACCATCTGC
GGTGATAAATTATCTCTGGCGGTGTTGACATAAATACCACTGGCGGTGATACTGAGCACATGTACTCAGATCCAATAGGAGGAACAA
TATGCGCGCGCTTGTTGTGATTCGTTTATCGCGTGTGACGGACGCCACCACCAGTCCCGAACGCCAATTAGAAAGCTGTCAACAACT
TTGTGCTCAACGTGGTTGGGATGTGGTGGGAGTGGCCGAAGACCTTGATGTTTCGGGCGCTGTTGACCCATTTGATCGCAAACGTCG
TCCTAATTTGGCGCGTTGGCTGGCCTTTGAAGAACAGCCTTTCGATGTAATTGTCGCATATCGCGTTGACCGTCTGACACGTTCCAT
TCGCCACCTGCAACAACTTGTGCATTGGGCGGAAGATCATAAGAAATTAGTTGTTTCGGCAACAGAGGCCCATTTTGACACCACCAC
ACCTTTCGCTGCTGTAGTTATTGCCCTGATGGGTACTGTCGCTCAAATGGAGCTCGAGGCAATTAAGGAACGCAATCGCTCCGCGGC
CCACTTTAACATTCGTGCTGGCAAGTATCGCGGCAGCTTACCCCCGTGGGGTTATCTCCCCACACGTGTAGATGGCGAATGGCGCCT
CGTCCCTGATCCGGTCCAACGCGAACGTATTCTGGAAGTCTACCATCGTGTAGTGGATAATCATGAACCCTTACACCTCGTTGCTCA
TGATCTCAATCGCCGCGGCGTTTTGAGCCCCAAAGATTATTTTGCCCAATTGCAGGGTCGTGAACCCCAAGGTCGCGAATGGTCCGC
CACAGCCTTAAAACGCTCAATGATTTCTGAAGCAATGCTTGGCTATGCAACGTTGAATGGCAAAACTGTTCGTGATGATGATGGCGC
GCCCTTGGTTCGCGCCGAACCAATTTTGACACGCGAACAATTAGAAGCTCTTCGTGCAGAACTGGTTAAAACATCGCGTGCCAAACC
GGCCGTTAGTACTCCTTCTCTTCTCCTTCGTGTCCTGTTTTGTGCTGTCTGTGGCGAACCTGCCTATAAATTTGCTGGTGGCGGGCG
CAAACATCCTCGTTATCGTTGTCGTAGCATGGGCTTTCCCAAACATTGTGGCAATGGTACTGTTGCTATGGCGGAATGGGATGCCTT
TTGTGAAGAACAAGTGTTAGACCTTCTGGGCGATGCAGAACGCCTCGAAAAGGTATGGGTCGCCGGGAGCGATAGCGCAGTGGAGCT
GGCTGAAGTAAATGCCGAACTCGTTGATTTAACTAGCTTGATTGGATCTCCTGCGTATCGCGCCGGATCGCCACAACGTGAGGCCTT
AGACGCGCGCATCGCTGCCTTAGCTGCCCGCCAGGAAGAACTCGAAGGTTTGGAAGCGCGTCCTAGCGGATGGGAATGGCGTGAAAC
TGGTCAACGCTTTGGAGATTGGTGGCGCGAACAAGATACGGCTGCCAAGAATACATGGCTGCGTTCCATGAATGTGCGTCTTACCTT
TGATGTGCGTGGTGGTTTAACCCGTACAATTGATTTTGGCGACCTGCAAGAATATGAACAACACCTGCGTCTGGGATCAGTCGTGGA
GCGCTTGCATACAGGCATGTCATAAGGCATCTAACTAAAAACACCCTAACGGCAAAATAATAAAAAAGCCGGATTAATAATCTGGCT
TTTTATATTCTCTCACTACCATCGGCGCTACGGCGTTTCACTTCTGAGTTCTGCAGCGGCCGCTACTAGTATGGTACCATTACGCCG
CTACGTCTTCCGTGCCGTCTTGAGCATCATCCTCATCATCATCCGTTGGGGGTTTAGCCCAAGTAATACTCGCACGTTTCTCAATCG
GGGTGCCTTGGCCGCGTCCGGTCGTTGATTTGGTCACCACAATTTTATCAACAAACAGGCCCACAAATACACGTTTATCATCCACAC
TTGCACGGCCCCACCAGCTTTTCGGACCTGTTGGGTCCGCATCCGCATCTTCCGGAAACCACTGATCCAGCGGCAATTTGGGTGCCT
CGGCCGCCTCGAGCTCTGCCAGACGTTCCTCGGCGCCCTGTTGACGTAAGGTTAATGCCGCTTGCTGTTTGCGAAAATGTTTACGAC
CGACCGGACCATCATATGCCCCGGCCGCACGATCCTCATACAGCTCTTCAAGGGCGTTCAGCGCATCCGCACGTTCGGCCACCAGAT
TGGCGCGCTCTCCTGATTTCTCCGGGGCTTCGGTAAGTTTACCAAAACGGCGCGCCGCTTCCCATAACAATGCCAGGGTTTCCTCAT
CACCCTCTGCATGACGAATTTTATTAAAGATACGCTCGGCTACAAATTTATCCAGCGCGGCCATAGACACATTACAAGTTCCCTCAT
GTTGGCCCGGCGCCGAGGGATCAACGACTTTGCGACGGCGACAACGATATGAATCTTTAATTGACTCCTCGCCACGTTTGGATGTCA
TAACCGCACCGCATTCACAATAAAGTTTATCCATCGCGCTTAAGATCGCCTGACCACGGCTCAGACCTTTACCACGACCGCGACCAT
CCAGCCAGGCTTGCAGTTCGTACCATTCCGCAGGTTCAATAATGGGGCCACAGTCCAGTTCAACAGGACGCAGGGTAATAGGATCAC
GTTGGATACGATACCCTTCGATTTTGGTCGTTGGGGTCCCATCTGGTTTCTTCTTATAAATGACTTCCGCAGCAAATCCGGCGATGC
GGGGATCGCGTAAAATGCGCATCACTGTGGCGGGGTCCCACGCCGAGCTCGCAGTTTTCTTGCCGATGGTTTCACCACGCGTTGGAA
CCGCATCCGCATCCATACGTTTGCACAAGCCGGTAATTGAACCAGGATGGATCGCCGCCTGGGAACCTGGTTTAAACGGCAGATGCT
TATGAGTTTTAATTTCGCGCCACCACCAACGAATGACATCTGGTTCAAATTCAAACGGGCCTGTCAGCGGGGTTGTTGAATGGGCCA
GTTTATTAATCACCACGTTAACCATGCGACCATTACGGGTAATTTCTTTGGTTTCGCTAACAAGCTCGAAGCCGTAAGGCGCTTTAC
CGCCCACATATCCACCCAGCTCACGTTGTAAATTCTTAGTATCCAGGATTTTGGCGCTCTTTAAACTGCTCTCCTTGTGTGAGGCAT
CCAGACGCATGATCAGGTGAATCAGGTCCATGACATTGCCTTGGCGAAACACACCCTCTTGGGTTGAGACGATGGTGACACCTAACG
CCAGTAACTCGCTTACGATGGGGATTGCATCCATCACTTTGAGACGAGAAAAGCGAGAAACATCGTACACGATAATCATATTTAAGC
GGCCCGCACGACACTCATTTAAAATACGCTCAAATTCAGGACGTTCGGCTGTACCAAAAGCGCTGGTCCCTGGAGCCTCTGAAAAGT
GACCCACAAAACGAAAGCGACCACCATCACGTTCCACCTCACGTTGCAGATCTGCGGCTTTATCCTCATTCGCGGAGCGTTGGGTTG
CGGGGGACGCGGCTGAGCTGTTTTCACGTTCACGAGATTGGCGATCGTAAGCACCCGCGTACGTGTCGCCGCTACCATGGTGATGGT
GATGGTGCATATTGTTCCTCCTATTGGATCTGAGTACATGCAACCATTATCACCGCCAGAGGTAAAATAGTCAACACGCGCGGTGTT
AGATATTTATCCCTTGCGGTGATATTGGTAACGAATCAGACAATTGACGGCGACACCATCGAATGGCGCAAACCTTTCGCGGTATGG
CATGATAGCGCCCGGAAGAGAGTCAATTCAGGGTGGTGAATGTATAAGAAGGAGATATACATATGAGCACAAAAAAGAAACCATTAA
CACAAGAGCAGCTTGAGGACGCACGTCGCCTTAAAGCAATTTATGAAAAAAAGAAAAATGAACTTGGCTTATCCCAGGAATCTGTCG
CAGACAAGATGGGGATGGGGCAGTCAGGCGTTGGTGCTTTATTTAATGGCATCAATGCATTAAATGCTTATAACGCCGCATTGCTTA
CAAAAATTCTCAAAGTTAGCGTTGAAGAATTTAGCCCTTCAATCGCCAGAGAAATCTACGAGATGTATGAAGCGGTTAGTATGCAGC
CGTCACTTAGAAGTGAGTATGAGTACCCTGTTTTTTCTCATGTTCAGGCAGGGATGTTCTCACCTGAGCTTAGAACCTTTACCAAAG
GTGATGCGGAGAGATGGGTAAGCACAACCAAAAAAGCCAGTGATTCTGCATTCTGGCTTGAGGTTGAAGGTAATTCCATGACCGCAC
CAACAGGCTCCAAGCCAAGCTTTCCTGACGGAATGTTAATTCTCGTTGACCCTGAGCAGGCTGTTGAGCCAGGTGATTTCTGCATAG
CCAGACTTGGGGGTGATGAGTTTACCTTCAAGAAACTGATCAGGGATAGCGGTCAGGTGTTTTTACAACCACTAAACCCACAGTACC
CAATGATCCCATGCAATGAGAGTTGTTCCGTTGTGGGGAAAGTTATCGCTAGTCAGTGGCCTGAAGAGACGTTTGGCTGACTCGAGT
CTGGTCCACCGCTGAGCAATAACTAGCATAACCCCTTGGGGCCTCTAAACGGGTCTTGAGGGGTTTTTTGCGATCTGAAGCGAACCA
TGACGTTAGTTCCTATTCCGAAGTTCCTATTCTCTAGAAAGTATAGGAACTTCTGATCGGCACGTAAGAGGTTCCAACTTTCACCAT
AATGAAATAAGATCACTACCGGGCGTATTTTTTGAGTTGTCGAGATTTTCAGGAGCTAAGGAAGCTAAAAACTTTTCGGATCAAGCT
ATGAAAAAAGAAGAAGAAACGAGAAACTAAACGTCCTTGCGGAAAATGAGACGTTGATCGGCACGTAAGAGGTTCCAACTTTCACCA
TAATGAAATAAGATCACTACCGGGCGTATTTTTTGAGTTGTCGAGATTTTCAGGAGCTAAGGAAGCTAAAATGGAGAAAAAAATCAC
TGGATATACCACCGTTGATATATCCCAATGGCATCGTAAAGAACATTTTGAGGCATTTCAGTCAGTTGCTCAATGTACCTATAACCA
GACCGTTCAGCTGGATATTACGGCCTTTTTAAAGACCGTAAAGAAAAATAAGCACAAGTTTTATCCGGCCTTTATTCACATTCTTGC
CCGCCTGATGAATGCTCATCCGGAATTACGTATGGCAATGAAAGACGGTGAGCTGGTGATATGGGATAGTGTTCACCCTTGTTACAC
CGTTTTCCATGAGCAAACTGAAACGTTTTCATCGCTCTGGAGTGAATACCACGACGATTTCCGGCAGTTTCTACACATATATTCGCA
AGATGTGGCGTGTTACGGTGAAAACCTGGCCTATTTCCCTAAAGGGTTTATTGAGAATATGTTTTTCGTCTCAGCCAATCCCTGGGT
GAGTTTCACCAGTTTTGATTTAAACGTGGCCAATATGGACAACTTCTTCGCCCCCGTTTTCACCATGGGCAAATATTATACGCAAGG
CGACAAGGTGCTGATGCCGCTGGCGATTCAGGTTCATCATGCCGTTTGTGATGGCTTCCATGTCGGCAGAATGCTTAATGAATTACA
ACAGTACTGCGATGAGTGGCAGGGGGGGGCGTAAGGCGCGCCATTTAAATGAAGTTCCTATTCCGAAGTTCCTATTCTCTAGAAAGT
ATAGGAACTTCGAAGCAGCTCTTGTTCAGAACGCTCGGTCTTGCACACCGGGCGTTTTTTCTTTGTGAGTCCAGCGTCGTGGTTTGT
CTGGTCAACCACCGCGGACTCAGTGGTGTACGGTACAAACCCAGTGCCCCAACTGGGGTAACCTAGGAGTTCTCTCAGTTGGGGGCG
TAACAATTGGGATCCACTGCTCTTTAACAATTTATCAGATCCAATAGGAGGAACAATATGAAAGGATCCAAAGGTGAAGAATTATTC
ACTGGAGTTGTCCCAATTCTTGTTGAATTAGATGGTGATGTTAATGGGCACAAATTTTCTGTCAGTGGAGAGGGTGAAGGTGATGCA
ACATACGGAAAACTTACCCTTAAATTTATTTGCACTACTGGAAAACTACCTGTTCCATGGCCAACACTTGTCACTACTTTCGCGTAT
GGTCTTCAATGCTTTGCGAGATACCCAGATCATATGAAACAGCATGACTTTTTCAAGAGTGCCATGCCCGAAGGTTATGTACAGGAA
AGAACTATATTTTTCAAAGATGACGGGAACTACAAGACACGTGCTGAAGTCAAGTTTGAAGGTGATACCCTTGTTAATAGAATCGAG
TTAAAAGGTATTGATTTTAAAGAAGATGGAAACATTCTTGGACACAAATTGGAATACAACTATAACTCACACAAAGTATACATCATG
GCAGACAAACAAAAGGATGGAATCAAAGTTAACTTCAAAATTAGACACAACATTGAAGATGGAAGCGTTCAACTAGCAGACCATTAT
CAACGAAATACTCCAATTGGCGATGGCCCTGTCCTTTTACCAGACAACCATTACCTGTCCACACAATCTGCCCTTTCGAAAGATCCC
AACGAAAAGAGAGACCACATGGTCCTTCTTGAGTTTGTAACAGCTGCTGGGATTACACATGGTATGGATGAATTGTACAAATAACGC
AAAAAACCCCGCCCCTGACAGGGGGGGGTTTTTTCGCTTTAGTTTCTCGTTTCTTCTTCTTCCTTCATAGCTTGATCCGAAAAGTTA
CTGTGGATAACTCTGTCAGGAAGCTTGGATCAACCGGTAGTTATCCAAAGAACAACTGTTGTTCAGTTTTTGAGTTGTGTATAACCC
CTCATTCTGATCCCAGCTTATACGGTCCAGGATCACCGATCATTCACAGTTAATGATCCTTTCCAGGTTGTTGATCTTAAAAGCCGG
ATCCTTGTTATCCACAGGGCAGTGCGATCCTAATAAGAGATCACAATAGAACAGATCTCTAAATAAATAGATCTTCTTTTTAATACC
CAGGATCCCAGGTCTTCACTTTTGAAAAGCATTGACAATTAATCATCGGCTCGTATAATGTGTGGAAGTGCGGGTGCCAGGGCGTGC
CCAGGGGCTCCCCGGGCGCGTACTCCGGCCGGCTTGTCGACGACGGCGGACTCCGTCGTCAGGATCATCCGGGCTCTAACTAAAAAC
ACCCTAACGGGTGTTTTTTCTTTTCTGGTCTCCCGAATTCGGACATAATCAAAAAAGCTTAATTAAGATCAATTTGATCTACATCTC
TTTAACCAACAATATGTAAGATCTCAACTATCGCATCCGTGGATTAATTCAATTATAACTTCTCTCTAACGCTGTGTATCGTAACGG
TAACACTGTAGACACTGCTCTTTAACAATTTATCAGATCCAATAGGAGGAACAATATGTCTACCAAAAAAAAACCACTTACACAGGA
ACAGTTGGAAGATGCCCGGCGTCTTAAGGCGATTTATGAGAAGAAGAAAAACGAATTAGGGCTGTCTCAAGAATCAGTGGCTGATAA
GATGGGTATGGGACAGTCCGGCGTCGGGGCGTTATTTAATGGTATAAACGCATTAAATGCCTACAACGCCGCTTTACTGACCAAAAT
TTTGAAAGTCTCCGTAGAAGAATTTAGTCCATCTATTGCTAGAGAGATATACGAGATGTACGAAGCCGTTAGTATGCAACCATCGCT
GCGTTCGGAGTATGAATATCCAGTCTTTTCTCATGTCCAAGCCGGAATGTTTTCCCCCGAGTTGCGGACATTTACGAAGGGAGACGC
AGAGCGTTGGGTGTCGACAACTAAGAAAGCCTCCGACTCTGCCTTCTGGCTGGAGGTAGAAGGAAACTCTATGACCGCCCCTACTGG
ATCTAAGCCTTCCTTCCCTGATGGGATGTTGATTCTGGTTGATCCTGAACAAGCGGTTGAGCCCGGGGATTTCTGCATTGCAAGATT
AGGCGGCGACGAATTTACTTTCAAAAAACTTATTCGCGATTCGGGACAAGTTTTCCTGCAACCACTGAACCCGCAATACCCAATGAT
CCCTTGTAATGAATCATGCAGTGTTGTTGGTAAGGTGATAGCTTCGCAGTGGCCAGAGGAGACCTTTGGGTAATACTAGAGAAAAAA
AAACCCCGCTTCGGCGGGGTTTTTTTTTGATCGCACGATCTGTATACTTATTTGAGTAAATTAACCCACGATCCCAGCCATTCTTCT
GCCGGATCTTCCGGAATGTCGTGATCAAGAATGTTGATCTTCAGTGTTTCGCCTGTCTGTTTTGCACCGGAATTTTTGAGTTCTGCC
TCGAGTTTATCGATAGCCCCACAAAAGGTGTCATATTCACGACTGCCAATACCGATTGCGCCAAAGCGGACTGCAGAAAGATCGGGC
TTCTGTTCCTGCAATGCTTCATAGAAAGGAGAAAGGTTGTCCGGAATATCTCCGGCACCGTGGGTGGAGCTGATAACCAGCCAGATC
CCTGAGGCAGGTAAATCTTCTAACAGCGGACCGTGCAGCGTTTCGGTGGTAAAACCCGCCTCTTCCAGCTTTTCAGCCAGGTGTTCT
GCTACATATTCGGCACCGCCGAGGGTGCTGCCGCTGATAAGAGTGATATCTGCCAT

TABLE 4
Sequences of plasmids

>pAJ27 (SEQ ID NO: 15)
AGATCAAAGGATCTTCTTGAGATCCTTTTTTTCTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCGGAGCCTATGGAAAAACGCCAGCAACGCGGCC
TTTTTACGGTTCCTGGCCTTTTGCTGGCCTTTTGCTCACATGTTCTTTCCTGCGTTATCCCCTGATTCTGTGGATAACCGTATTACCGC
CTTTGAGTGAGCTGATACCGCTCGCCGCAGCCGAACGACCGAGCGCAGCGAGTCAGTGAGCGAGGAAGCGGAAGAGCGCCTGATGCGGT
ATTTTCTCCTTACGCATCTGTGCGGTATTTCACACCGCATATATGGTGCACTCTCAGTACAATCTGCTCTGATGCCGCATAGTTAAGCC
AGTATACACTCCGCTATCGCTACGTGACTGGGTCATGGCTGCGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTTTTCACCGTCATCACCGAAACGCGCG
AGGCAGCTGCGGTAAAGCTCATCAGCGTGGTCGTGAAGCGATTCACAGATGTCTGCCTGTTCATCCGCGTCCAGCTCGTTGAGTTTCTC
CAGAAGCGTTAATGTCTGGCTTCTGATAAAGCGGGCCATGTTAAGGGCGGTTTTTTCCTGTTTGGTCACTGATGCCTCCGTGTAAGGGG
GATTTCTGTTCATGGGGGTAATGATACCGATGAAACGAGAGAGGATGCTCACGATACGGGTTACTGATGATGAACATGCCCGGTTACTG
GAACGTTGTGAGGGTAAACAACTGGCGGTATGGATGCGGCGGGACCAGAGAAAAATCACTCAGGGTCAATGCCAGCGCTTCGTTAATAC
AGATGTAGGTGTTCCACAGGGTAGCCAGCAGCATCCTGCGATGCAGATCCGGAACATAATGGTGCAGGGCGCTGACTTCCGCGTTTCCA
GACTTTACGAAACACGGAAACCGAAGACCATTCATGTTGTTGCTCAGGTCGCAGACGTTTTGCAGCAGCAGTCGCTTCACGTTCGCTCG
CGTATCGGTGATTCATTCTGCTAACCAGTAAGGCAACCCCGCCAGCCTAGCCGGGTCCTCAACGACAGGAGCACGATCATGCTAGTCAT
GCCCCGCGCCCACCGGAAGGAGCTGACTGGGTTGAAGGCTCTCAAGGGCATCGGTCGAGATCCCGGTGCCTAATGAGTGAGCTAACTTA
CATTAATTGCGTTGCGCATGTACTACCGGATATAGTTCCTCCTTTCAGCAAAAAACCCCTCAAGACCCGTTTAGAGGCCCCAAGGGGTT
ATGCTAGTTATTGCTCAGCGGTGGCAGCAGCCTAGGTTAATTAAGCTGCGCTAGTAGACGAGTCCATGTGCTGGCGTTCAAATTTCGCA
GCAGCGGTTTCTTTACCAGACTCGAGTCAGCCAAACGTCTCTTCAGGCCACTGACTAGCGATAACTTTCCCCACAACGGAACAACTCTC
ATTGCATGGGATCATTGGGTACTGTGGGTTTAGTGGTTGTAAAAACACCTGACCGCTATCCCTGATCAGTTTCTTGAAGGTAAACTCAT
CACCCCCAAGTCTGGCTATGCAGAAATCACCTGGCTCAACAGCCTGCTCAGGGTCAACGAGAATTAACATTCCGTCAGGAAAGCTTGGC
TTGGAGCCTGTTGGTGCGGTCATGGAATTACCTTCAACCTCAAGCCAGAATGCAGAATCACTGGCTTTTTTGGTTGTGCTTACCCATCT
CTCCGCATCACCTTTGGTAAAGGTTCTAAGCTCAGGTGAGAACATCCCTGCCTGAACATGAGAAAAAACAGGGTACTCATACTCACTTC
TAAGTGACGGCTGCATACTAACCGCTTCATACATCTCGTAGATTTCTCTGGCGATTGAAGGGCTAAATTCTTCAACGCTAACTTTGAGA
ATTTTTGTAAGCAATGCGGCGTTATAAGCATTTAATGCATTGATGCCATTAAATAAAGCACCAACGCCTGACTGCCCCATCCCCATCTT
GTCTGCGACAGATTCCTGGGATAAGCCAAGTTCATTTTTCTTTTTTTCATAAATTGCTTTAAGGCGACGTGCGTCCTCAAGCTGCTCTT
GTGTTAATGGTTTCTTTTTTGTGCTCATATGTATATCTCCTTCTTATACATTCACCACCCTGAATTGACTCTCTTCCGGGCGCTATCAT
GCCATACCGCGAAAGGTTTGCGCCATTCGATGGTGTCGCCGTCAATTGTCTGATTCGTTACCAATATCACCGCAAGGGATAAATATCTA
ACACCGCGCGTGTTGACTATTTTACCTCTGGCGGTGATAATGGTTGCATGTACTCAGATCCAATAGGAGGAACAATATGGACACGTACG
CGGGTGCTTACGACCGTCAGTCGCGCGAGCGCGAAAATTCGAGCGCAGCAAGCCCAGCGACACAGCGTAGCGCCGATGGTAGTGAGAGA
ATATAAAAAGCCAGATTATTAATCCGGCTTTTTTATTATTTTGCCAGGTGGCACTTTTCGGGGAAATGTGCGCGGAACCCCTATTTGTT
TATTTTTCTAAATACATTCAAATATGTATCCGCTCATGAATTAATTCTTAGAAAAACTCATCGAGCATCAAATGAAACTGCAATTTATT
CATATCAGGATTATCAATACCATATTTTTGAAAAAGCCGTTTCTGTAATGAAGGAGAAAACTCACCGAGGCAGTTCCATAGGATGGCAA
GATCCTGGTATCGGTCTGCGATTCCGACTCGTCCAACATCAATACAACCTATTAATTTCCCCTCGTCAAAAATAAGGTTATCAAGTGAG
AAATCACCATGAGTGACGACTGAATCCGGTGAGAATGGCAAAAGTTTATGCATTTCTTTCCAGACTTGTTCAACAGGCCAGCCATTACG
CTCGTCATCAAAATCACTCGCATCAACCAAACCGTTATTCATTCGTGATTGCGCCTGAGCGAGACGAAATACGCGATCGCTGTTAAAAG
GACAATTACAAACAGGAATCGAATGCAACCGGCGCAGGAACACTGCCAGCGCATCAACAATATTTTCACCTGAATCAGGATATTCTTCT
AATACCTGGAATGCTGTTTTCCCGGGGATCGCAGTGGTGAGTAACCATGCATCATCAGGAGTACGGATAAAATGCTTGATGGTCGGAAG
AGGCATAAATTCCGTCAGCCAGTTTAGTCTGACCATCTCATCTGTAACATCATTGGCAACGCTACCTTTGCCATGTTTCAGAAACAACT
CTGGCGCATCGGGCTTCCCATACAATCGATAGATTGTCGCACCTGATTGCCCGACATTATCGCGAGCCCATTTATACCCATATAAATCA
GCATCCATGTTGGAATTTAATCGCGGCCTAGAGCAAGACGTTTCCCGTTGAATATGGCTCATAACACCCCTTGTATTACTGTTTATGTA
AGCAGACAGTTTTATTGTTCATGACCAAAATCCCTTAACGTGAGTTTTCGTTCCACTGAGCGTCAGACCCCGTAGAAA
>pAJ35 (SEQ ID NO: 16)
AGATCAAAGGATCTTCTTGAGATCCTTTTTTTCTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCGGAGCCTATGGAAAAACGCCAGCAACGCGGCC
TTTTTACGGTTCCTGGCCTTTTGCTGGCCTTTTGCTCACATGTTCTTTCCTGCGTTATCCCCTGATTCTGTGGATAACCGTATTACCGC
CTTTGAGTGAGCTGATACCGCTCGCCGCAGCCGAACGACCGAGCGCAGCGAGTCAGTGAGCGAGGAAGCGGAAGAGCGCCTGATGCGGT
ATTTTCTCCTTACGCATCTGTGCGGTATTTCACACCGCATATATGGTGCACTCTCAGTACAATCTGCTCTGATGCCGCATAGTTAAGCC
AGTATACACTCCGCTATCGCTACGTGACTGGGTCATGGCTGCGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTTTTCACCGTCATCACCGAAACGCGCG
AGGCAGCTGCGGTAAAGCTCATCAGCGTGGTCGTGAAGCGATTCACAGATGTCTGCCTGTTCATCCGCGTCCAGCTCGTTGAGTTTCTC
CAGAAGCGTTAATGTCTGGCTTCTGATAAAGCGGGCCATGTTAAGGGCGGTTTTTTCCTGTTTGGTCACTGATGCCTCCGTGTAAGGGG
GATTTCTGTTCATGGGGGTAATGATACCGATGAAACGAGAGAGGATGCTCACGATACGGGTTACTGATGATGAACATGCCCGGTTACTG
GAACGTTGTGAGGGTAAACAACTGGCGGTATGGATGCGGCGGGACCAGAGAAAAATCACTCAGGGTCAATGCCAGCGCTTCGTTAATAC
AGATGTAGGTGTTCCACAGGGTAGCCAGCAGCATCCTGCGATGCAGATCCGGAACATAATGGTGCAGGGCGCTGACTTCCGCGTTTCCA
GACTTTACGAAACACGGAAACCGAAGACCATTCATGTTGTTGCTCAGGTCGCAGACGTTTTGCAGCAGCAGTCGCTTCACGTTCGCTCG
CGTATCGGTGATTCATTCTGCTAACCAGTAAGGCAACCCCGCCAGCCTAGCCGGGTCCTCAACGACAGGAGCACGATCATGCTAGTCAT
GCCCCGCGCCCACCGGAAGGAGCTGACTGGGTTGAAGGCTCTCAAGGGCATCGGTCGAGATCCCGGTGCCTAATGAGTGAGCTAACTTA
CATTAATTGCGTTGCGCATGTACTACCGGATATAGTTCCTCCTTTCAGCAAAAAACCCCTCAAGACCCGTTTAGAGGCCCCAAGGGGTT
ATGCTAGTTATTGCTCAGCGGTGGCAGCAGCCTAGGTTAATTAAGCTGCGCTAGTAGACGAGTCCATGTGCTGGCGTTCAAATTTCGCA
GCAGCGGTTTCTTTACCAGACTCGAGTCAGCCAAACGTCTCTTCAGGCCACTGACTAGCGATAACTTTCCCCACAACGGAACAACTCTC
ATTGCATGGGATCATTGGGTACTGTGGGTTTAGTGGTTGTAAAAACACCTGACCGCTATCCCTGATCAGTTTCTTGAAGGTAAACTCAT
CACCCCCAAGTCTGGCTATGCAGAAATCACCTGGCTCAACAGCCTGCTCAGGGTCAACGAGAATTAACATTCCGTCAGGAAAGCTTGGC
TTGGAGCCTGTTGGTGCGGTCATGGAATTACCTTCAACCTCAAGCCAGAATGCAGAATCACTGGCTTTTTTGGTTGTGCTTACCCATCT
CTCCGCATCACCTTTGGTAAAGGTTCTAAGCTCAGGTGAGAACATCCCTGCCTGAACATGAGAAAAAACAGGGTACTCATACTCACTTC
TAAGTGACGGCTGCATACTAACCGCTTCATACATCTCGTAGATTTCTCTGGCGATTGAAGGGCTAAATTCTTCAACGCTAACTTTGAGA
ATTTTTGTAAGCAATGCGGCGTTATAAGCATTTAATGCATTGATGCCATTAAATAAAGCACCAACGCCTGACTGCCCCATCCCCATCTT
GTCTGCGACAGATTCCTGGGATAAGCCAAGTTCATTTTTCTTTTTTTCATAAATTGCTTTAAGGCGACGTGCGTCCTCAAGCTGCTCTT
GTGTTAATGGTTTCTTTTTTGTGCTCATATGTATATCTCCTTCTTATACATTCACCACCCTGAATTGACTCTCTTCCGGGCGCTATCAT
GCCATACCGCGAAAGGTTTGCGCCATTCGATGGTGTCGCCGTCAATTGTCTGATTCGTTACCAATATCACCGCAAGGGATAAATATCTA
ACACCGCGCGTGTTGACTATTTTACCTCTGGCGGTGATAATGGTTGCATGTACTCAGATCCAATAGAAGGAACAATATGGACACGTACG
CGGGTGCTTACGACCGTCAGTCGCGCGAGCGCGAAAATTCGAGCGCAGCAAGCCCAGCGACACAGCGTAGCGCCAACGAAGACAAGGCG
GCCGACCTTCAGCGCGAAGTCGAGCGCGACGGGGGCCGGTTCAGGTTCGTCGGGCATTTCAGCGAAGCGCCGGGCACGTCGGCGTTCGG
GACGGCGGAGCGCCCGGAGTTCGAACGCATCCTGAACGAATGCCGCGCCGGGCGGCTCAACATGATCATTGTCTATGACGTGTCGCGCT
TCTCGCGCCTGAAGGTCATGGACGCGATTCCGATTGTCTCGGAATTGCTCGCCCTGGGCGTGACGATTGTTTCCACTCAGGAAGGCGTC
TTCCGGCAGGGAAACGTCATGGACCTGATTCACCTGATTATGCGGCTCGACGCGTCGCACAAAGAATCTTCGCTGAAGTCGGCGAAGAT
TCTCGACACGAAGAACCTTCAGCGCGAATTGGGCGGGTACGTCGGCGGGAAGGCGCCTTACGGCTTCGAGCTTGTTTCGGAGACGAAGG
AGATCACGCGCAACGGCCGAATGGTCAATGTCGTCATCAACAAGCTTGCGCACTCGACCACTCCCCTTACCGGACCCTTCGAGTTCGAG
CCCGACGTAATCCGGTGGTGGTGGCGTGAGATCAAGACGCACAAACACCTTCCCTTCAAGCCGGGCAGTCAAGCCGCCATTCACCCGGG
CAGCATCACGGGGCTTTGTAAGCGCATGGACGCTGACGCCGTGCCGACCCGGGGCGAGACGATTGGGAAGAAGACCGCTTCAAGCGCCT
GGGACCCGGCAACCGTTATGCGAATCCTTCGGGACCCGCGTATTGCGGGCTTCGCCGCTGAGGTGATCTACAAGAAGAAGCCGGACGGC
ACGCCGACCACGAAGATTGAGGGTTACCGCATTCAGCGCGACCCGATCACGCTCCGGCCGGTCGAGCTTGATTGCGGACCGATCATCGA
GCCCGCTGAGTGGTATGAGCTTCAGGCGTGGTTGGACGGCAGGGGGCGCGGCAAGGGGCTTTCCCGGGGGCAAGCCATTCTGTCCGCCA
TGGACAAGCTGTACTGCGAGTGTGGCGCCGTCATGACTTCGAAGCGCGGGGAAGAATCGATCAAGGACTCTTACCGCTGCCGTCGCCGG
AAGGTGGTCGACCCGTCCGCACCTGGGCAGCACGAAGGCACGTGCAACGTCAGCATGGCGGCACTCGACAAGTTCGTTGCGGAACGCAT
CTTCAACAAGATCAGGCACGCCGAAGGCGACGAAGAGACGTTGGCGCTTCTGTGGGAAGCCGCCCGACGCTTCGGCAAGCTCACTGAGG
CGCCTGAGAAGAGCGGCGAACGGGCGAACCTTGTTGCGGAGCGCGCCGACGCCCTGAACGCCCTTGAAGAGCTGTACGAAGACCGCGCG
GCAGGCGCGTACGACGGACCCGTTGGCAGGAAGCACTTCCGGAAGCAACAGGCAGCGCTGACGCTCCGGCAGCAAGGGGCGGAAGAGCG
GCTTGCCGAACTTGAAGCCGCCGAAGCCCCGAAGCTTCCCCTTGACCAATGGTTCCCCGAAGACGCCGACGCTGACCCGACCGGCCCTA
AGTCGTGGTGGGGGCGCGCGTCAGTAGACGACAAGCGCGTGTTCGTCGGGCTCTTCGTAGACAAGATCGTTGTCACGAAGTCGACTACG
GGCAGGGGGCAGGGAACGCCCATCGAGAAGCGCGCTTCGATCACGTGGGCGAAGCCGCCGACCGACGACGACGAAGACGACGCCCAGGA
CGGCACGGAAGACGTAGCGGCGTAGAACTCAGAAGTGAAACGCCGTAGCGCCGATGGTAGTGAGAGAATATAAAAAGCCAGATTATTAA
TCCGGCTTTTTTATTATTTTGCCAGGTGGCACTTTTCGGGGAAATGTGCGCGGAACCCCTATTTGTTTATTTTTCTAAATACATTCAAA
TATGTATCCGCTCATGAATTAATTCTTAGAAAAACTCATCGAGCATCAAATGAAACTGCAATTTATTCATATCAGGATTATCAATACCA
TATTTTTGAAAAAGCCGTTTCTGTAATGAAGGAGAAAACTCACCGAGGCAGTTCCATAGGATGGCAAGATCCTGGTATCGGTCTGCGAT
TCCGACTCGTCCAACATCAATACAACCTATTAATTTCCCCTCGTCAAAAATAAGGTTATCAAGTGAGAAATCACCATGAGTGACGACTG
AATCCGGTGAGAATGGCAAAAGTTTATGCATTTCTTTCCAGACTTGTTCAACAGGCCAGCCATTACGCTCGTCATCAAAATCACTCGCA
TCAACCAAACCGTTATTCATTCGTGATTGCGCCTGAGCGAGACGAAATACGCGATCGCTGTTAAAAGGACAATTACAAACAGGAATCGA
ATGCAACCGGCGCAGGAACACTGCCAGCGCATCAACAATATTTTCACCTGAATCAGGATATTCTTCTAATACCTGGAATGCTGTTTTCC
CGGGGATCGCAGTGGTGAGTAACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTCTGGCGCATCGGGCTTCCCATA
CAATCGATAGATTGTCGCACCTGATTGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTATGTAAGCAGACAGTTTTATTGTTCAT
GACCAAAATCCCTTAACGTGAGTTTTCGTTCCACTGAGCGTCAGACCCCGTAGAAA
>pAJ144 (SEQ ID NO: 17)
GGGCCGCCGGCGTTGTGGATACCACGCGGAAAACTTGGCCCTCACTGACAGATGAGGGGCGGACGTTGACACTTGAGGGGCCGACTCAC
CCGGCGCGGCGTTGACAGATGAGGGGCAGGCTCGATTTCGGCCGGCGACGTGGAGCTGGCCAGCCTCGCAAATCGGCGAAAACGCCTGA
TTTTACGCGAGTTTCCCACAGATGATGTGGACAAGCCTGGGGATAAGTGCCCTGCGGTATTGACACTTGAGGGGCGCGACTACTGACAG
ATGAGGGGCGCGATCCTTGACACTTGAGGGGCAGAGTGATGACAGATGAGGGGCGCACCTATTGACATTTGAGGGGCTGTCCACAGGCA
GAAAATCCAGCATTTGCAAGGGTTTCCGCCCGTTTTTCGGCCACCGCTAACCTGTCTTTTAACCTGCTTTTAAACCAATATTTATAAAC
CTTGTTTTTAACCAGGGCTGCGCCCTGGCGCGTGACCGCGCACGCCGAAGGGGGGTGCCCCCCCTTCTCGAACCCTCCCGGCCCGCTAA
CGCGGCACCCCATCCCCCCAGGGGCTGCGCCCCTCGGCCGCGAACGACCTCACCCCAAAAATGGCAGCCACGTAGAAAGCCAGTCCGCA
GAAACGGTGCTGACCCCGGATGAATGTCAGCTACTGGGCTATCTGGACAAGGGAAAACGCAAGCGCAAAGAGAAAGCAGGTAGCTTGCA
GTGGGCTTACATGGCGATAGCTAGACTGGGCGGTITTATGGACAGCAAGCGAACCGGAATTGCCAGCTGGGGCGCCCTCTGGTAAGGTT
GGGAAGCCCTGCAAAGTAAACTGGATGGCTTTCTTGCCGCCAAGGATCTGATGGCGCAGGGGATCAAGATCGACGGATCGATCCGGGGA
ATTAATTCCGGGGCAATCCCGCAAGGAGGGTGAATGAATCGGACGTTTGACCGGAAGGCATACAGGCAAGAACTGATCGACGCGGGGTT
TTCCGCCGAGGATGCCGAAACCATCGCAAGCCGCACCGTCATGCGTGCGCCCCGCGAAACCTTCCAGTCCGTCGGCTCGATGGTCCAGC
AAGCTACGGCCAAGATCGAGCGCGACAGCGTGCAACTGGCTCCCCCTGCCCTGCCCGCGCCATCGGCCGCCGTGGAGCGTTCGCGTCGT
CTCGAACAGGAGGCGGCAGGTTTGGCGAAGTCGATGACCATCGACACGCGAGGAACTATGACGACCAAGAAGCGAAAAACCGCCGGCGA
GGACCTGGCAAAACAGGTCAGCGAGGCCAAGCAGGCCGCGTTGCTGAAACACACGAAGCAGCAGATCAAGGAAATGCAGCTTTCCTTGT
TCGATATTGCGCCGTGGCCGGACACGATGCGAGCGATGCCAAACGACACGGCCCGCTCTGCCCTGTTCACCACGCGCAACAAGAAAATC
CCGCGCGAGGCGCTGCAAAACAAGGTCATTTTCCACGTCAACAAGGACGTGAAGATCACCTACACCGGCGTCGAGCTGCGGGCCGACGA
TGACGAACTGGTGTGGCAGCAGGTGTTGGAGTACGCGAAGCGCACCCCTATCGGCGAGCCGATCACCTTCACGTTCTACGAGCTTTGCC
AGGACCTGGGCTGGTCGATCAATGGCCGGTATTACACGAAGGCCGAGGAATGCCTGTCGCGCCTACAGGCGACGGCGATGGGCTTCACG
TCCGACCGCGTTGGGCACCTGGAATCGGTGTCGCTGCTGCACCGCTTCCGCGTCCTGGACCGTGGCAAGAAAACGTCCCGTTGCCAGGT
CCTGATCGACGAGGAAATCGTCGTGCTGTTTGCTGGCGACCACTACACGAAATTCATATGGGAGAAGTACCGCAAGCTGTCGCCGACGG
CCCGACGGATGTTCGACTATTTCAGCTCGCACCGGGAGCCGTACCCGCTCAAGCTGGAAACCTTCCGCCTCATGTGCGGATCGGATTCC
ACCCGCGTGAAGAAGTGGCGCGAGCAGGTCGGCGAAGCCTGCGAAGAGTTGCGAGGCAGCGGCCTGGTGGAACACGCCTGGGTCAATGA
TGACCTGGTGCATTGCAAACGCTAGGGCCTTGTGGGGTCAGTTCCGGCTGGGGGTTCAGCAGCCACCTGCATCGCAAGCTAGCTTGCTA
GAGGGTCAGCTTTATGCTTGTAAACCGTTTTGTGAAAAAATTTTTAAAATAAAAAAGGGGACCTCTAGGGTCCCCAATTAATTAGTAAT
ATAATCTATTAAAGGTCATTCAAAAGGTCATCCACCGGATCAATTCCCCTGCTCGCGCAGGCTGGGTGCCAAGCTCTCGGGTAACATCA
AGGCCCGATCCTTGGTAGTCGGCAAATAATGTCTAACAATTCGTTCAAGCCGACGCCGCTTCGCGGCGCGGCTTAACTCAAGCGTTAGA
TGCACTAAGCACATAATTGCTCACAGCCAAACTATCAGGTCAAGTCTGCTTTTATTATTTTTAAGCGTGCATAATAAGCCCTACACAAA
TTGGGAGATATATCATGAAAGGCTGGCTTTTTCTTGTTATCGCAATAGTTGGCGAAGTAATCGCAACATCCGCATTAAAATCTAGCGAG
GGCTTTACTAAGCTGATCCGGTGGATGACCTTTTGAATGACCTTTAATAGATTATATTACTAATTAATTGGGGACCCTAGAGGTCCCCT
TTTTTATTTTAAAAATTTTTTCACAAAACGGTTTACAAGCATAAAGCTGACTCTAGCTAGAGGATCTTCGAATGCATCGCGCGCACCGT
ACGTCTCGAGGTCGACTTAATTTTTAAAGTATGGGCAATCAATTGCTCCTGTTAAAATTGCTTTAGAAATACTTTGGCAGCGGTTTGTT
GTATTGAGTTTCATTTGCGCATTGGTTAAATGGAAAGTGACAGTACGCTCACTGCAGCCTAATATTTTTGAAATATCCCAAGAGCTTTT
TCCTTCGCATGCCCACGCTAAACATTCTTTTTCTCTTTTGGTTAAATCGTTGTTTGATTTATTATTTGCTATATTTATTTTTCGATAAT
TATCAACTAGAGAAGGAACAATTAATGGTATGTTCATACACGCATGTAAAAATAAACTATCTATATAGTTGTCTTTTTCTGAATGTGCA
AAACTAAGCATTCCGAAGCCATTGTTAGCCGTATGAATAGGGAAACTAAACCCAGTGATAAGACCTGATGTTTTCGCTTCTTTAATTAC
ATTTGGAGATTTTTTATTTACAGCATTGTTTTCAAATATATTCCAATTAATTGGTGAATGATTGGAGTTAGAATAATCTACTATAGGAT
CATATTTTATTAAATTAGCGTCATCATAATATTGCCTCCATTTTTTAGGGTAATTATCTAGAATTGAAATATCAGATITAACCATAGAA
TGAGGATAAATGATCGCGAGTAAATAATATTCACAATGTACCATTITAGTCATATCAGATAAGCATTGATTAATATCATTATTGCTTCT
ACAAGCTTTAATTTTATTAATTATTCTGTATGTGTCGTCGGCATTTATGTTTTTCATACCCATCTCTTTATCCTTACCTATTGTTTGTC
GCAAGTTTTGCGTGTTATATATCATTAAAACGGTAATGGATTGACATTTGAATTCACATAAGCACCTGTAGGATCGTACAGGTTTAGCG
AAGAAAATGGTTTGTTATAGTCGAATAACTGCAGGAGGTACCCGGGGATCCTCTAGAAATAATTTTGTTTAACTTTAAGAAGGAGATAT
ACATATGCGGGGTTCTCATCATCATCATCATCATGGTATGGCTAGCATGACTGGTGGACAGCAAATGGGTCGGGATCTGTACGACGATG
ACGATAAGGATCCGATGGCCTCCTCCGAGGACGTCATCAAGGAGTTCATGCGCTTCAAGGTGCGCATGGAGGGCTCCGTGAACGGCCAC
GAGTTCGAGATCGAGGGCGAGGGCGAGGGCCGCCCCTACGAGGGCACCCAGACCGCCAAGCTGAAGGTGACCAAGGGCGGCCCCCTGCC
CTTCGCCTGGGACATCCTGTCCCCTCAGTTCCAGTACGGCTCCAAGGCCTACGTGAAGCACCCCGCCGACATCCCCGACTACTTGAAGC
TGTCCTTCCCCGAGGGCTTCAAGTGGGAGCGCGTGATGAACTTCGAGGACGGCGGCGTGGTGACCGTGACCCAGGACTCCTCCCTGCAG
GACGGCGAGTTCATCTACAAGGTGAAGCTGCGCGGCACCAACTTCCCCTCCGACGGCCCCGTAATGCAGAAGAAGACCATGGGCTGGGA
GGCCTCCACCGAGCGGATGTACCCCGAGGACGGCGCCCTGAAGGGCGAGATCAAGATGAGGCTGAAGCTGAAGGACGGCGGCCACTACG
ACGCCGAGGTCAAGACCACCTACATGGCCAAGAAGCCCGTGCAGCTGCCCGGCGCCTACAAGACCGACATCAAGCTGGACATCACCTCC
CACAACGAGGACTACACCATCGTGGAACAGTACGAGCGCGCCGAGGGCCGCCACTCCACCGGCGCCTAAGAATTCCTGCAGGATATCTG
GATCCACGAAGCTTCCCATGGTGACGTCACCGGTTCTAGATACCTAGGTGAGCTCTGGTACCGCGGCCGATCTTCGTCCACGCCGAGCC
CTATGCAGTCTCCGATTATGTTAACCAGTATGTCGGTACGCACTCTATTCGCCTGCCCAAGGGCGGGCCCCCGGCAGGCAGGCTGCACC
ACAGAATCTTCGGATGCCTCGACCTGTGTCGAATCAGCTACGGCGGTAGCGTGAGGGTAATCTCGCCTGGATTAGAGACCTGTTATCAT
CTGCAAATAATACTCAAAGGCCATTGCCTGTGGCGTGGCCATGGCCAGGAGCACTATTTTGCGCCGGGCGAACTATTGCTGCTCAATCC
GGATGACCAAGCCGACCTGACCTATTCAGAAGATTGCGAGAAATTTATCGTTAAATTGCCCTCAGTGGTCCTTGATCGGGCATGCAGTG
ACAACAATTGGCACAAGCCGAGGGAGGGTATCCGTTTCGCCGCGCGACACAATCTCCAGCAACTCGATGGCTTTATCAATCTACTCGGG
TTAGTTTGTGACGAAGCGGAACATACAAAGTCGATGCCTCGGGTCCAAGAGCACTATGCGGGGATCATCGCTTCCAAGCTGCTCGAAAT
GCTGGGCAGCAATGTCAGCCGTGAAATTTTCAGCAAAGGTAACCCGTCTTTCGAGCGAGTCGTTCAATTCATTGAGGAGAATCTCAAAC
GGAATATCAGCCTTGAGCGGTTAGCGGAGCTGGCGATGATGAGTCCACGCTCGCTCTACAATTTGTTCGAGAAGCATGCCGGCACCACG
CCGAAGAACTACATCCGCAACCGCAAGCTCGAAAGCATCCGCGCCTGCTTGAACGATCCCAGTGCCAATGTGCGTAGTATAACTGAGAT
AGCCCTAGACTACGGCTTCTTACATTTGGGACGCTTCGCTGAAAACTATAGGAGCGCGTTCGGCGAGTTGCCTTCCGACACCCTGCGTC
AATGCAAAAAGGAAGTGGCTTGATTACGAACGTAGCCGAAGAAGGGATGGGTTGGCATCGCCCGGCTTTCTTAGACACTCTCCAAGCTC
TGAAATAGCGTTTTACAAACTCCACTGGCTATAGTGCCGGCGTTTGCCCGCGCTCTCCGTGGCCGCGTCCCAATGCAGGTCGGGTTCCT
CGACCCGAAGTGCCCTTGGGCATCTCCACTTCAGCGTCTGACCTTGCTGGCCAGTTCGCCGCCGATGCCGAAGTGGCCCTTGGCCATCT
CCGTGATAATCACTCGCACGCTGGTCAGCGGCGCATCCAGGGAGCGCGAGATGGCCTCGCTGACTTCCCGAATGAGGGTTTCCTTCTGC
TCGTCGCTGCGGCCTTCAAGGATGTGGATCTGCTGGCCGAAAGGGGGATGTGCTGCAAGGCGATTAAGTTGGGTAACGCCAGGGTTTTC
CCAGTCACGACGTTGTAAAACGACGGCCAGTGAATTAATTCTTGAAGACGAAAGGGCCTCGTGATACGCCTATTITTATAGGTTAATGT
CATGATAATAATGGTTTCTTAGAGCTTACGGCCAGCCTCGCAGAGCAGGATTCCCGTTGAGCACCGCCAGGTGCGAATAAGGGACAGTG
AAGAAGGAACACCCGCTCGCGGGTGGGCCTACTTCACCTATCCTGCCCGGCTGACGCCGTTGGATACACCAAGGAAAGTCTACACGAAC
CCTTTGGCAAAATCCTGTATATCGTGCGAAAAAGGATGGATATACCGAAAAAATCGCTATAATGACCCCGAAGCAGGGTTATGCAGCGG
AAAAGATCCGTCGGCTGCAGCGATCCGTCGATCTATCTCATCTGCGCAAGGCAGAACGTGAAGACGGCCGCCCTGGACCTCGCCCGCGA
GCGCCAGCGCACGAGGCCGGCGCGCGGACCCGCCGCGGCCCACGAGCGGACGCCGCAGCAGGAGCGCCAGAAGGCCGCCAGAGAGGCCG
AGCGCGGCGTGAGGCTTGGACGCTAGGGCAGGGCATGAAAAAGCCCGTAGCGGGCGCTACGGGGCTCTGACGCGGTGGAAAGGGGGAGG
GGATGTTGTCTACATGGCTCTGCTGTAGTGAGTGGGTTGCGCTCCGGCAGCGGTCCTGATCAATCGTCACCCTTTCTCGGTCCTTCAAC
GTTCCTGACAACGAGCCTCCTTTTCGCCAATCCATCGACAATCACCGCGAGTCCCTGCTCGAACGCTGCGTCCGGACCGGCTTCGTCGA
AGGCGTCTATCGCGGCCCGCAACAGCGGCGAGAGCGGAGCCTGTTCAACGGTGCCGCCGCGCTCGCCGGACTCGCTGTCGCCGGCCTGC
TCCTCAAGCACGGCCCCAACAGTGAAGTAGCTGATTGTCATCAGCGCATTGACGGCGTCCCCGGCCGAAAAACCCGCCTCGCAGAGGAA
GCGAAGCTGCGCGTCGGCCGTTTCCATCTGCGGTGCGCCCGGTCGCGTGCCGGCATGGATGCGCGCGCCATCGCGGTAGGCGAGCAGCG
CCTGCCTGAAGCTGCGGGCATTCCCAGTCAGAAATGAGCGCCAGTCGTCGTCGGCTCTCGGCACCGAAGTGCTATGATTCTCCGCCAGC
ATGGCTTCGGCCAGTGCGTCGAGCAGCGCCCGCTTGTTCCTGAAGTGCCAGTAAAGCGCCGGCTGCTGAACCCCCAACCGTTCCGCCAG
TTTGCGTGTCGTCAGACCGTCTACGCCGACCTCGTTCAACAGGTCTAGGGCGGCACGGATCACTGTATTCGGCTGCAACTTTGTCATGC
TTGACACTTTATCACTGATAAACATAATATGTCCACCAACTTATCAGTGATAAAGAATCCGCGCGTTCAATCGGACCAGCGGAGGCTGG
TCCGGAGGCCAGACGTGAAACCCAACATACCCCTGATCGTAATTCTGAGCACTGTCGCGCTCGACGCTGTCGGCATCGGCCTGATTATG
CCGGTGCTGCCGGGCCTCCTGCGCGATCTGGTTCACTCGAACGACGTCACCGCCCACTATGGCATTCTGCTGGCGCTGTATGCGTTGGT
GCAATTTGCCTGCGCACCTGTGCTGGGCGCGCTGTCGGATCGTTTCGGGCGGCGGCCAATCTTGCTCGTCTCGCTGGCCGGCGCCACTG
TCGACTACGCCATCATGGCGACAGCGCCTTTCCTTTGGGTTCTCTATATCGGGCGGATCGTGGCCGGCATCACCGGGGCGACTGGGGCG
GTAGCCGGCGCTTATATTGCCGATATCACTGATGGCGATGAGCGCGCGCGGCACTTCGGCTTCATGAGCGCCTGTTTCGGGTTCGGGAT
GGTCGCGGGACCTGTGCTCGGTGGGCTGATGGGCGGTTTCTCCCCCCACGCTCCGTTCTTCGCCGCGGCAGCCTTGAACGGCCTCAATT
TCCTGACGGGCTGTTTCCTTTTGCCGGAGTCGCACAAAGGCGAACGCCGGCCGTTACGCCGGGAGGCTCTCAACCCGCTCAGCTTCGTT
CGGTGGGCCCGGGGCATGACCGTCGTCGCCGCCCTGATGGCGGTCTTCTTCATCATGCAACTTGTCGGACAGGTGCCGGCCGCGCTTTG
GGTCATTTTCGGCGAGGATCGCTTTCACTGGGACGCGACCACGATCGGCATTTCGCTTGCCGCATTTGGCATTCTGCATTCACTCGCCC
AGGCAATGATCACCGGCCCTGTAGCCGCCCGGCTCGGCGAAAGGCGGGCACTCATGCTCGGAATGATTGCCGACGGCACAGGCTACATC
CTGCTTGCCTTCGCGACACGGGGATGGATGGCGTTCCCGATCATGGTCCTGCTTGCTTCGGGTGGCATCGGAATGCCGGCGCTGCAAGC
AATGTTGTCCAGGCAGGTGGATGAGGAACGCCAGGGGCAGCTGCAAGGCTCACTGGCGGCGCTCACCAGCCTGACCTCGATCGTCGGAC
CCCTCCTCTTCACGGCGATCTATGCGGCTTCTATAACAACGTGGAACGGGTGGGCATGGATTGCAGGCGCTGCCCTCTACTTGCTCTGC
CTGCCGGCGCTGCGTCGCGGGCTTTGGAGCGGCGCAGGGCAACGAGCCGATCGCTGATCGTGGAAACGATAGGGACGGATCGCTGCAGC
GCAAACTATTAACTGGCGAACTACTTACTCTAGCTTCCCGGCAACAATTAATAGACTGGATGGAGGCGGATAAAGTTGCAGGACCACTT
CTGCGCTCGGCCCTTCCGGCTGGCTGGTTTATTGCTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGCAACTATGGATGAACGAAATAGACAGATCGCTGAGA
TAGGTGCCTCACTGATTAAGCATTGGTAACTGTCAGACCAAGTTTACTCATATATACTTTAGATTGATTTAAAACTTCATTTTTAATTT
AAAAGGATCTAGGTGAAGATCCTTTTTGATAATCTCATGACCAAAATCCCTTAACGTGAGTTTTCGTTCCACTGAGCGTCAGACCCCGT
AGAAAAGATCAAAGGATCTTCTTGAGATCCTTTTTTTCTGCGGGGGATCAGGACCGCTGCCGGAGCGCAACCCACTCACTACAGCAGAG
CCATGTA
>pAJ144 (SEQ ID NO: 23)
GGGCCGCCGGCGTTGTGGATACCACGCGGAAAACTTGGCCCTCACTGACAGATGAGGGGCGGACGTTGACACTTGAGGGGCCGACTCAC
CCGGCGCGGCGTTGACAGATGAGGGGCAGGCTCGATTTCGGCCGGCGACGTGGAGCTGGCCAGCCTCGCAAATCGGCGAAAACGCCTGA
TTTTACGCGAGTTTCCCACAGATGATGTGGACAAGCCTGGGGATAAGTGCCCTGCGGTATTGACACTTGAGGGGCGCGACTACTGACAG
ATGAGGGGCGCGATCCTTGACACTTGAGGGGCAGAGTGATGACAGATGAGGGGCGCACCTATTGACATTTGAGGGGCTGTCCACAGGCA
GAAAATCCAGCATTTGCAAGGGTTTCCGCCCGTTTTTCGGCCACCGCTAACCTGTCTTTTAACCTGCTTTTAAACCAATATTTATAAAC
CTTGTTTTTAACCAGGGCTGCGCCCTGGCGCGTGACCGCGCACGCCGAAGGGGGGTGCCCCCCCTTCTCGAACCCTCCCGGCCCGCTAA
CGCGGCACCCCATCCCCCCAGGGGCTGCGCCCCTCGGCCGCGAACGACCTCACCCCAAAAATGGCAGCCACGTAGAAAGCCAGTCCGCA
GAAACGGTGCTGACCCCGGATGAATGTCAGCTACTGGGCTATCTGGACAAGGGAAAACGCAAGCGCAAAGAGAAAGCAGGTAGCTTGCA
GTGGGCTTACATGGCGATAGCTAGACTGGGCGGTITTATGGACAGCAAGCGAACCGGAATTGCCAGCTGGGGCGCCCTCTGGTAAGGTT
GGGAAGCCCTGCAAAGTAAACTGGATGGCTTTCTTGCCGCCAAGGATCTGATGGCGCAGGGGATCAAGATCGACGGATCGATCCGGGGA
ATTAATTCCGGGGCAATCCCGCAAGGAGGGTGAATGAATCGGACGTTTGACCGGAAGGCATACAGGCAAGAACTGATCGACGCGGGGTT
TTCCGCCGAGGATGCCGAAACCATCGCAAGCCGCACCGTCATGCGTGCGCCCCGCGAAACCTTCCAGTCCGTCGGCTCGATGGTCCAGC
AAGCTACGGCCAAGATCGAGCGCGACAGCGTGCAACTGGCTCCCCCTGCCCTGCCCGCGCCATCGGCCGCCGTGGAGCGTTCGCGTCGT
CTCGAACAGGAGGCGGCAGGTTTGGCGAAGTCGATGACCATCGACACGCGAGGAACTATGACGACCAAGAAGCGAAAAACCGCCGGCGA
GGACCTGGCAAAACAGGTCAGCGAGGCCAAGCAGGCCGCGTTGCTGAAACACACGAAGCAGCAGATCAAGGAAATGCAGCTTTCCTTGT
TCGATATTGCGCCGTGGCCGGACACGATGCGAGCGATGCCAAACGACACGGCCCGCTCTGCCCTGTTCACCACGCGCAACAAGAAAATC
CCGCGCGAGGCGCTGCAAAACAAGGTCATTTTCCACGTCAACAAGGACGTGAAGATCACCTACACCGGCGTCGAGCTGCGGGCCGACGA
TGACGAACTGGTGTGGCAGCAGGTGTTGGAGTACGCGAAGCGCACCCCTATCGGCGAGCCGATCACCTTCACGTTCTACGAGCTTTGCC
AGGACCTGGGCTGGTCGATCAATGGCCGGTATTACACGAAGGCCGAGGAATGCCTGTCGCGCCTACAGGCGACGGCGATGGGCTTCACG
TCCGACCGCGTTGGGCACCTGGAATCGGTGTCGCTGCTGCACCGCTTCCGCGTCCTGGACCGTGGCAAGAAAACGTCCCGTTGCCAGGT
CCTGATCGACGAGGAAATCGTCGTGCTGTTTGCTGGCGACCACTACACGAAATTCATATGGGAGAAGTACCGCAAGCTGTCGCCGACGG
CCCGACGGATGTTCGACTATTTCAGCTCGCACCGGGAGCCGTACCCGCTCAAGCTGGAAACCTTCCGCCTCATGTGCGGATCGGATTCC
ACCCGCGTGAAGAAGTGGCGCGAGCAGGTCGGCGAAGCCTGCGAAGAGTTGCGAGGCAGCGGCCTGGTGGAACACGCCTGGGTCAATGA
TGACCTGGTGCATTGCAAACGCTAGGGCCTTGTGGGGTCAGTTCCGGCTGGGGGTTCAGCAGCCACCTGCATCGCAAGCTAGCTTGCTA
GAGGGTCAGCTTTATGCTTGTAAACCGTTTTGTGAAAAAATTTTTAAAATAAAAAAGGGGACCTCTAGGGTCCCCAATTAATTAGTAAT
ATAATCTATTAAAGGTCATTCAAAAGGTCATCCACCGGATCAATTCCCCTGCTCGCGCAGGCTGGGTGCCAAGCTCTCGGGTAACATCA
AGGCCCGATCCTTGGTAGTCGGCAAATAATGTCTAACAATTCGTTCAAGCCGACGCCGCTTCGCGGCGCGGCTTAACTCAAGCGTTAGA
TGCACTAAGCACATAATTGCTCACAGCCAAACTATCAGGTCAAGTCTGCTTTTATTATTTTTAAGCGTGCATAATAAGCCCTACACAAA
TTGGGAGATATATCATGAAAGGCTGGCTTTTTCTTGTTATCGCAATAGTTGGCGAAGTAATCGCAACATCCGCATTAAAATCTAGCGAG
GGCTTTACTAAGCTGATCCGGTGGATGACCTTTTGAATGACCTTTAATAGATTATATTACTAATTAATTGGGGACCCTAGAGGTCCCCT
TTTTTATTTTAAAAATTTTTTCACAAAACGGTTTACAAGCATAAAGCTGACTCTAGCTAGAGGATCTTCGAATGCATCGCGCGCACCGT
ACGTCTCGAGGTCGACTTAATTTTTAAAGTATGGGCAATCAATTGCTCCTGTTAAAATTGCTTTAGAAATACTTTGGCAGCGGTTTGTT
GTATTGAGTTTCATTTGCGCATTGGTTAAATGGAAAGTGACAGTACGCTCACTGCAGCCTAATATTTTTGAAATATCCCAAGAGCTTTT
TCCTTCGCATGCCCACGCTAAACATTCTTTTTCTCTTTTGGTTAAATCGTTGTTTGATTTATTATTTGCTATATTTATTTTTCGATAAT
TATCAACTAGAGAAGGAACAATTAATGGTATGTTCATACACGCATGTAAAAATAAACTATCTATATAGTTGTCTTTTTCTGAATGTGCA
AAACTAAGCATTCCGAAGCCATTGTTAGCCGTATGAATAGGGAAACTAAACCCAGTGATAAGACCTGATGTTTTCGCTTCTTTAATTAC
ATTTGGAGATTTTTTATTTACAGCATTGTTTTCAAATATATTCCAATTAATTGGTGAATGATTGGAGTTAGAATAATCTACTATAGGAT
CATATTTTATTAAATTAGCGTCATCATAATATTGCCTCCATTTTTTAGGGTAATTATCTAGAATTGAAATATCAGATTTAACCATAGAA
TGAGGATAAATGATCGCGAGTAAATAATATTCACAATGTACCATTITAGTCATATCAGATAAGCATTGATTAATATCATTATTGCTTCT
ACAAGCTTTAATTTTATTAATTATTCTGTATGTGTCGTCGGCATTTATGTTTTTCATACCCATCTCTTTATCCTTACCTATTGTTTGTC
GCAAGTTTTGCGTGTTATATATCATTAAAACGGTAATGGATTGACATTTGATTCTAATAAATTGGATTTTTGTCACACTATTGTATCGC
TGGGAATACAATTACTTAACATAAGCACCTGTAGGATCGTACAGGTTTAGCGAAGAAAATGGTTTGTTATAGTCGAATAACTGCAGGAG
GTACCCGGGGATCCTCTAGAAATAATTTTGTTTAACTTTAAGAAGGAGATATACATATGCGGGGTTCTCATCATCATCATCATCATGGT
ATGGCTAGCATGACTGGTGGACAGCAAATGGGTCGGGATCTGTACGACGATGACGATAAGGATCCGATGGCCTCCTCCGAGGACGTCAT
CAAGGAGTTCATGCGCTTCAAGGTGCGCATGGAGGGCTCCGTGAACGGCCACGAGTTCGAGATCGAGGGCGAGGGCGAGGGCCGCCCCT
ACGAGGGCACCCAGACCGCCAAGCTGAAGGTGACCAAGGGCGGCCCCCTGCCCTTCGCCTGGGACATCCTGTCCCCTCAGTTCCAGTAC
GGCTCCAAGGCCTACGTGAAGCACCCCGCCGACATCCCCGACTACTTGAAGCTGTCCTTCCCCGAGGGCTTCAAGTGGGAGCGCGTGAT
GAACTTCGAGGACGGCGGCGTGGTGACCGTGACCCAGGACTCCTCCCTGCAGGACGGCGAGTTCATCTACAAGGTGAAGCTGCGCGGCA
CCAACTTCCCCTCCGACGGCCCCGTAATGCAGAAGAAGACCATGGGCTGGGAGGCCTCCACCGAGCGGATGTACCCCGAGGACGGCGCC
CTGAAGGGCGAGATCAAGATGAGGCTGAAGCTGAAGGACGGCGGCCACTACGACGCCGAGGTCAAGACCACCTACATGGCCAAGAAGCC
CGTGCAGCTGCCCGGCGCCTACAAGACCGACATCAAGCTGGACATCACCTCCCACAACGAGGACTACACCATCGTGGAACAGTACGAGC
GCGCCGAGGGCCGCCACTCCACCGGCGCCTAAGAATTCCTGCAGGATATCTGGATCCACGAAGCTTCCCATGGTGACGTCACCGGTTCT
AGATACCTAGGTGAGCTCTGGTACCGCGGCCGATCTTCGTCCACGCCGAGCCCTATGCAGTCTCCGATTATGTTAACCAGTATGTCGGT
ACGCACTCTATTCGCCTGCCCAAGGGCGGGCCCCCGGCAGGCAGGCTGCACCACAGAATCTTCGGATGCCTCGACCTGTGTCGAATCAG
CTACGGCGGTAGCGTGAGGGTAATCTCGCCTGGATTAGAGACCTGTTATCATCTGCAAATAATACTCAAAGGCCATTGCCTGTGGCGTG
GCCATGGCCAGGAGCACTATTTTGCGCCGGGCGAACTATTGCTGCTCAATCCGGATGACCAAGCCGACCTGACCTATTCAGAAGATTGC
GAGAAATTTATCGTTAAATTGCCCTCAGTGGTCCTTGATCGGGCATGCAGTGACAACAATTGGCACAAGCCGAGGGAGGGTATCCGTTT
CGCCGCGCGACACAATCTCCAGCAACTCGATGGCTTTATCAATCTACTCGGGTTAGTTTGTGACGAAGCGGAACATACAAAGTCGATGC
CTCGGGTCCAAGAGCACTATGCGGGGATCATCGCTTCCAAGCTGCTCGAAATGCTGGGCAGCAATGTCAGCCGTGAAATTTTCAGCAAA
GGTAACCCGTCTTTCGAGCGAGTCGTTCAATTCATTGAGGAGAATCTCAAACGGAATATCAGCCTTGAGCGGTTAGCGGAGCTGGCGAT
GATGAGTCCACGCTCGCTCTACAATTTGTTCGAGAAGCATGCCGGCACCACGCCGAAGAACTACATCCGCAACCGCAAGCTCGAAAGCA
TCCGCGCCTGCTTGAACGATCCCAGTGCCAATGTGCGTAGTATAACTGAGATAGCCCTAGACTACGGCTTCTTACATTTGGGACGCTTC
GCTGAAAACTATAGGAGCGCGTTCGGCGAGTTGCCTTCCGACACCCTGCGTCAATGCAAAAAGGAAGTGGCTTGATTACGAACGTAGCC
GAAGAAGGGATGGGTTGGCATCGCCCGGCTTTCTTAGACACTCTCCAAGCTCTGAAATAGCGTTTTACAAACTCCACTGGCTATAGTGC
CGGCGTTTGCCCGCGCTCTCCGTGGCCGCGTCCCAATGCAGGTCGGGTTCCTCGACCCGAAGTGCCCTTGGGCATCTCCACTTCAGCGT
CTGACCTTGCTGGCCAGTTCGCCGCCGATGCCGAAGTGGCCCTTGGCCATCTCCGTGATAATCACTCGCACGCTGGTCAGCGGCGCATC
CAGGGAGCGCGAGATGGCCTCGCTGACTTCCCGAATGAGGGTTTCCTTCTGCTCGTCGCTGCGGCCTTCAAGGATGTGGATCTGCTGGC
CGAAAGGGGGATGTGCTGCAAGGCGATTAAGTTGGGTAACGCCAGGGTTTTCCCAGTCACGACGTTGTAAAACGACGGCCAGTGAATTA
ATTCTTGAAGACGAAAGGGCCTCGTGATACGCCTATTTTTATAGGTTAATGTCATGATAATAATGGTTTCTTAGAGCTTACGGCCAGCC
TCGCAGAGCAGGATTCCCGTTGAGCACCGCCAGGTGCGAATAAGGGACAGTGAAGAAGGAACACCCGCTCGCGGGTGGGCCTACTTCAC
CTATCCTGCCCGGCTGACGCCGTTGGATACACCAAGGAAAGTCTACACGAACCCTTTGGCAAAATCCTGTATATCGTGCGAAAAAGGAT
GGATATACCGAAAAAATCGCTATAATGACCCCGAAGCAGGGTTATGCAGCGGAAAAGATCCGTCGGCTGCAGCGATCCGTCGATCTATC
TCATCTGCGCAAGGCAGAACGTGAAGACGGCCGCCCTGGACCTCGCCCGCGAGCGCCAGCGCACGAGGCCGGCGCGCGGACCCGCCGCG
GCCCACGAGCGGACGCCGCAGCAGGAGCGCCAGAAGGCCGCCAGAGAGGCCGAGCGCGGCGTGAGGCTTGGACGCTAGGGCAGGGCATG
AAAAAGCCCGTAGCGGGCGCTACGGGGCTCTGACGCGGTGGAAAGGGGGAGGGGATGTTGTCTACATGGCTCTGCTGTAGTGAGTGGGT
TGCGCTCCGGCAGCGGTCCTGATCAATCGTCACCCTTTCTCGGTCCTTCAACGTTCCTGACAACGAGCCTCCTTTTCGCCAATCCATCG
ACAATCACCGCGAGTCCCTGCTCGAACGCTGCGTCCGGACCGGCTTCGTCGAAGGCGTCTATCGCGGCCCGCAACAGCGGCGAGAGCGG
AGCCTGTTCAACGGTGCCGCCGCGCTCGCCGGACTCGCTGTCGCCGGCCTGCTCCTCAAGCACGGCCCCAACAGTGAAGTAGCTGATTG
TCATCAGCGCATTGACGGCGTCCCCGGCCGAAAAACCCGCCTCGCAGAGGAAGCGAAGCTGCGCGTCGGCCGTTTCCATCTGCGGTGCG
CCCGGTCGCGTGCCGGCATGGATGCGCGCGCCATCGCGGTAGGCGAGCAGCGCCTGCCTGAAGCTGCGGGCATTCCCAGTCAGAAATGA
GCGCCAGTCGTCGTCGGCTCTCGGCACCGAAGTGCTATGATTCTCCGCCAGCATGGCTTCGGCCAGTGCGTCGAGCAGCGCCCGCTTGT
TCCTGAAGTGCCAGTAAAGCGCCGGCTGCTGAACCCCCAACCGTTCCGCCAGTTTGCGTGTCGTCAGACCGTCTACGCCGACCTCGTTC
AACAGGTCTAGGGCGGCACGGATCACTGTATTCGGCTGCAACTTTGTCATGCTTGACACTTTATCACTGATAAACATAATATGTCCACC
AACTTATCAGTGATAAAGAATCCGCGCGTTCAATCGGACCAGCGGAGGCTGGTCCGGAGGCCAGACGTGAAACCCAACATACCCCTGAT
CGTAATTCTGAGCACTGTCGCGCTCGACGCTGTCGGCATCGGCCTGATTATGCCGGTGCTGCCGGGCCTCCTGCGCGATCTGGTTCACT
CGAACGACGTCACCGCCCACTATGGCATTCTGCTGGCGCTGTATGCGTTGGTGCAATTTGCCTGCGCACCTGTGCTGGGCGCGCTGTCG
GATCGTTTCGGGCGGCGGCCAATCTTGCTCGTCTCGCTGGCCGGCGCCACTGTCGACTACGCCATCATGGCGACAGCGCCTTTCCTTTG
GGTTCTCTATATCGGGCGGATCGTGGCCGGCATCACCGGGGCGACTGGGGGGTAGCCGGCGCTTATATTGCCGATATCACTGATGGCGA
TGAGCGCGCGCGGCACTTCGGCTTCATGAGCGCCTGTTTCGGGTTCGGGATGGTCGCGGGACCTGTGCTCGGTGGGCTGATGGGCGGTT
TCTCCCCCCACGCTCCGTTCTTCGCCGCGGCAGCCTTGAACGGCCTCAATTTCCTGACGGGCTGTTTCCTTTTGCCGGAGTCGCACAAA
GGCGAACGCCGGCCGTTACGCCGGGAGGCTCTCAACCCGCTCAGCTTCGTTCGGTGGGCCCGGGGCATGACCGTCGTCGCCGCCCTGAT
GGCGGTCTTCTTCATCATGCAACTTGTCGGACAGGTGCCGGCCGCGCTTTGGGTCATTTTCGGCGAGGATCGCTTTCACTGGGACGCGA
CCACGATCGGCATTTCGCTTGCCGCATTTGGCATTCTGCATTCACTCGCCCAGGCAATGATCACCGGCCCTGTAGCCGCCCGGCTCGGC
GAAAGGCGGGCACTCATGCTCGGAATGATTGCCGACGGCACAGGCTACATCCTGCTTGCCTTCGCGACACGGGGATGGATGGCGTTCCC
GATCATGGTCCTGCTTGCTTCGGGTGGCATCGGAATGCCGGCGCTGCAAGCAATGTTGTCCAGGCAGGTGGATGAGGAACGCCAGGGGC
AGCTGCAAGGCTCACTGGCGGCGCTCACCAGCCTGACCTCGATCGTCGGACCCCTCCTCTTCACGGCGATCTATGCGGCTTCTATAACA
ACGTGGAACGGGTGGGCATGGATTGCAGGCGCTGCCCTCTACTTGCTCTGCCTGCCGGCGCTGCGTCGCGGGCTTTGGAGCGGCGCAGG
GCAACGAGCCGATCGCTGATCGTGGAAACGATAGGGACGGATCGCTGCAGCGCAAACTATTAACTGGCGAACTACTTACTCTAGCTTCC
CGGCAACAATTAATAGACTGGATGGAGGCGGATAAAGTTGCAGGACCACTTCTGCGCTCGGCCCTTCCGGCTGGCTGGTTTATTGCTGA
TAAATCTGGAGCCGGTGAGCGTGGGTCTCGCGGTATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAGGTGCCTCACTGATTAAGCATTGGTAACTGTCAGAC
CAAGTTTACTCATATATACTTTAGATTGATTTAAAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAAAAGATCAAAGGATCTTCTTGAGATCCTTTTTTTC
TGCGGGGGATCAGGACCGCTGCCGGAGCGCAACCCACTCACTACAGCAGAGCCATGTA
>pAJ157 (SEQ ID NO: 18)
TTTCCATAGGCTCCGCCCCCCTGACAAGCATCACGAAATCTGACGCTCAAATCAGTGGTGGCGAAACCCGACAGGACTATAAAGATACC
AGGCGTTTCCCCCTGGCGGCTCCCTCGTGCGCTCTCCTGTTCCTGCCTTTCGGTTTACCGGTGTCATTCCGCTGTTATGGCCGCGTTTG
TCTCATTCCACGCCTGACACTCAGTTCCGGGTAGGCAGTTCGCTCCAAGCTGGACTGTATGCACGAACCCCCCGTTCAGTCCGACCGCT
GCGCCTTATCCGGTAACTATCGTCTTGAGTCCAACCCGGAAAGACATGCAAAAGCACCACTGGCAGCAGCCACTGGTAATTGATTTAGA
GGAGTTAGTCTTGAAGTCATGCGCCGGTTAAGGCTAAACTGAAAGGACAAGTTTTGGTGACTGCGCTCCTCCAAGCCAGTTACCTCGGT
TCAAAGAGTTGGTAGCTCAGAGAACCTTCGAAAAACCGCCCTGCAAGGCGGTTTTTTCGTTTTCAGAGCAAGAGATTACGCGCAGACCA
AAACGATCTCAAGAAGATCATCTTATTAATCAGATAAAATATTTCTAGATTTCAGTGCAATTTATCTCTTCAAATGTAGCACCTGAAGT
CAGCCCCATACGAAGGCTCTCAAGGGCATCGGTCGAGATCCCGGTGCCTAATGAGTGAGCTAACTTACATTAATTGCGTTGCGCTCACT
GCCCGCTTTCCAGTCGGGAAACCTGTCGTGCCAGCTGCATTAATGAATCGGCCAACGCGCGGGGAGAGGCGGTTTGCGTATTGGGCGCC
AGGGTGGTTTTTCTTTTCACCAGTGAGACGGGCAACAGCTGATTGCCCTTCACCGCCTGGCCCTGAGAGAGTTGCAGCAAGCGGTCCAC
GCTGGTTTGCCCCAGCAGGCGAAAATCCTGTTTGATGGTGGTTAACGGCGGGATATAACATGAGCTGTCTTCGGTATCGTCGTATCCCA
CTACCGAGATGTCCGCACCAACGCGCAGCCCGGACTCGGTAATGGCGCGCATTGCGCCCAGCGCCATCTGATCGTTGGCAACCAGCATC
GCAGTGGGAACGATGCCCTCATTCAGCATTTGCATGGTTTGTTGAAAACCGGACATGGCACTCCAGTCGCCTTCCCGTTCCGCTATCGG
CTGAATTTGATTGCGAGTGAGATATTTATGCCAGCCAGCCAGACGCAGACGCGCCGAGACAGAACTTAATGGGCCCGCTAACAGCGCGA
TTTGCTGGTGACCCAATGCGACCAGATGCTCCACGCCCAGTCGCGTACCGTCTTCATGGGAGAAAATAATACTGTTGATGGGTGTCTGG
TCAGAGACATCAAGAAATAACGCCGGAACATTAGTGCAGGCAGCTTCCACAGCAATGGCATCCTGGTCATCCAGCGGATAGTTAATGAT
CAGCCCACTGACGCGTTGCGCGAGAAGATTGTGCACCGCCGCTTTACAGGCTTCGACGCCGCTTCGTTCTACCATCGACACCACCACGC
TGGCACCCAGTTGATCGGCGCGAGATTTAATCGCCGCGACAATTTGCGACGGCGCGTGCAGGGCCAGACTGGAGGTGGCAACGCCAATC
AGCAACGACTGTTTGCCCGCCAGTTGTTGTGCCACGCGGTTGGGAATGTAATTCAGCTCCGCCATCGCCGCTTCCACTTTTTCCCGCGT
TTTCGCAGAAACGTGGCTGGCCTGGTTCACCACGCGGGAAACGGTCTGATAAGAGACACCGGCATACTCTGCGACATCGTATAACGTTA
CTGGTTTCACATTCACCACCCTGAATTGACTCTCTTCCGGGCGCTATCATGCCATACCGCGAAAGGTTTTGCGCCATTCGATGGTGTCC
GGGATCTCGACGCTCTCCCTTATGCGACTCCTGCATTAGGAAGCAGCCCAGTAGTAGGTTGAGGCCGTTGAGCACCGCCGCCGCAAGGA
ATGGTGCATGCTCCCGGCCGCCATGGCGGCCGCGGGAATTCGATTTGGGCCTTTTGACAATAATCATCGGCTCGTATAATGTGTGGAAT
TGTGAGCGGATAACAATTGGGATCCGCCTTTAACAATTTATCAGATCCAATAGGAGGAGATCTATGGATAGCACTGAGAACGTCATCAA
GCCCTTCATGCGCTTCAAGGTGCACATGGAGGGCTCCGTGAACGGCCACGAGTTCGAGATCGAGGGCGTGGGCGAGGGCAAGCCCTACG
AGGGCACCCAGACCGCCAAGCTGCAAGTGACCAAGGGCGGCCCCCTGCCCTTCGCCTGGGACATCCTGTCCCCCCAGTTCTTCTACGGC
TCCAAGGCGTACATCAAGCACCCCGCCGACATCCCCGACTACCTCAAGCAGTCCTTCCCCGAGGGCTTCAAGTGGGAGCGCGTGATGAA
CTTCGAGGACGGCGGCGTGGTGACCGTGACCCAGGACTCCTCCCTGCAGGACGGCACCCTCATCTACCACGTGAAGTTCATCGGCGTGA
ACTTCCCCTCCGACGGCCCCGTAATGCAGAAGAAGACTCTGGGCTGGGAGCCCTCCACTGAGCGCAACTACCCCCGCGACGGCGTGCTG
AAGGGCGAGAACCACATGGCGCTGAAGCTGAAGGGCGGCGGCCACTACCTGTGTGAGTTCAAGTCCATCTACATGGCCAAGAAGCCCGT
GAAGCTGCCCGGCTACCACTACGTGGACTACAAGCTCGACATCACCTCCCACAACGAGGACTACACCGTGGTGGAGCAGTACGAGCGCG
CCGAGGCCCGCCACCACCTGTTCCAGTAGGTCGACAAGCTTGCGGCCGCACTCGAGCACCACCACCACCACCACCACCACTAATTGATT
AATACCTAGGCTGCTAAACAAAGCCCGAAAGGAAGCTGAGTTGGCTGCTGCCACCGCTGAGCAATAACTAGCATAACCCCTTGGGGCCT
CTAAACGGGTATTGAGGGGTTTTTTGCTGGCACGATGGCATGAGATTATCAAAAAGGATCTTCACCTAGATCCTTTTAAATTAAAAATG
AAGTTTTAAATCAATCTAAAGTATATATGAGTAAACTTGGTCTGACAGTTACCAATGCTTAATCAGTGAGGCACCTATCTCAGCGATCT
GTCTATTTCGTTCATCCATAGTTGCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGTTAATAGTTTGCGCAACGTTGTTG
CCATTGCTACAGGCATCGTGGTGTCACGCTCGTCGTTTGGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTGACTGGTGAGTACTCAACCAAGTCATTCTGAG
AATAGTGTATGCGGCGACCGAGTTGCTCTTGCCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCAGTTCGATGTAACCCACTCGTGCACCCAACTG
ATCTTCAGCATCTTTTACTTTCACCAGCGTTTCTGGGTGAGCAAAAACAGGAAGGCAAAATGCCGCAAAAAAGGGAATAAGGGCGACAC
GGAAATGTTGAATACTCATACTCTTCCTTTTTCAATCATGATTGAAGCATTTATCAGGGTTATTGTCTCATGAGCGGATACATATTTGA
ATGTATTTAGAAAAATAAACAAATAGGTCATGACCAAAATCCCTTAACGGCAAAAGCACCGCCGGACATCAGCGCTAGCGGAGTGTATA
CTGGCTTACTATGTTGGCACTGATGAGGGTGTCAGTGAAGTGCTTCATGTGGCAGGAGAAAAAAGGCTGCACCGGTGCGTCAGCAGAAT
ATGTGATACAGGATATATTCCGCTTCCTCGCTCACTGACTCGCTACGCTCGGTCGTTCGACTGCGGCGAGCGGAAATGGCTTACGAACG
GGGCGGAGATTTCCTGGAAGATGCCAGGAAGATACTTAACAGGGAAGTGAGAGGGCCGCGGCAAAGCCGTT
>pAJ194 (SEQ ID NO: 19)
AGATCAAAGGATCTTCTTGAGATCCTTTTTTTCTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCGGAGCCTATGGAAAAACGCCAGCAACGCGGCC
TTTTTACGGTTCCTGGCCTTTTGCTGGCCTTTTGCTCACATGTTCTTTCCTGCGTTATCCCCTGATTCTGTGGATAACCGTATTACCGC
CTTTGAGTGAGCTGATACCGCTCGCCGCAGCCGAACGACCGAGCGCAGCGAGTCAGTGAGCGAGGAAGCGGAAGAGCGCCTGATGCGGT
ATTTTCTCCTTACGCATCTGTGCGGTATTTCACACCGCATATATGGTGCACTCTCAGTACAATCTGCTCTGATGCCGCATAGTTAAGCC
AGTATACACTCCGCTATCGCTACGTGACTGGGTCATGGCTGCGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTTTTCACCGTCATCACCGAAACGCGCG
AGGCAGCTGCGGTAAAGCTCATCAGCGTGGTCGTGAAGCGATTCACAGATGTCTGCCTGTTCATCCGCGTCCAGCTCGTTGAGTTTCTC
CAGAAGCGTTAATGTCTGGCTTCTGATAAAGCGGGCCATGTTAAGGGCGGTTTTTTCCTGTTTGGTCACTGATGCCTCCGTGTAAGGGG
GATTTCTGTTCATGGGGGTAATGATACCGATGAAACGAGAGAGGATGCTCACGATACGGGTTACTGATGATGAACATGCCCGGTTACTG
GAACGTTGTGAGGGTAAACAACTGGCGGTATGGATGCGGCGGGACCAGAGAAAAATCACTCAGGGTCAATGCCAGCGCTTCGTTAATAC
AGATGTAGGTGTTCCACAGGGTAGCCAGCAGCATCCTGCGATGCAGATCCGGAACATAATGGTGCAGGGCGCTGACTTCCGCGTTTCCA
GACTTTACGAAACACGGAAACCGAAGACCATTCATGTTGTTGCTCAGGTCGCAGACGTTTTGCAGCAGCAGTCGCTTCACGTTCGCTCG
CGTATCGGTGATTCATTCTGCTAACCAGTAAGGCAACCCCGCCAGCCTAGCCGGGTCCTCAACGACAGGAGCACGATCATGCTAGTCAT
GCCCCGCGCCCACCGGAAGGAGCTGACTGGGTTGAAGGCTCTCAAGGGCATCGGTCGAGATCCCGGTGCCTAATGAGTGAGCTAACTTA
CATTAATTGCGTTGCGCTCACTGCCCGCTTTCCAGTCGGGAAACCTGTCGTGCCAGCTGCATTAATGAATCGGCCAACGCGCGGGGAGA
GGCGGTTTGCGTATTGGGCGCCAGGGTGGTTTTTCTTTTCACCAGTGAGACGGGCAACAGCTGATTGCCCTTCACCGCCTGGCCCTGAG
AGAGTTGCAGCAAGCGGTCCACGCTGGTTTGCCCCAGCAGGCGAAAATCCTGTTTGATGGTGGTTAACGGCGGGATATAACATGAGCTG
TCTTCGGTATCGTCGTATCCCACTACCGAGATGTCCGCACCAACGCGCAGCCCGGACTCGGTAATGGCGCGCATTGCGCCCAGCGCCAT
CTGATCGTTGGCAACCAGCATCGCAGTGGGAACGATGCCCTCATTCAGCATTTGCATGGTTTGTTGAAAACCGGACATGGCACTCCAGT
CGCCTTCCCGTTCCGCTATCGGCTGAATTTGATTGCGAGTGAGATATTTATGCCAGCCAGCCAGACGCAGACGCGCCGAGACAGAACTT
AATGGGCCCGCTAACAGCGCGATTTGCTGGTGACCCAATGCGACCAGATGCTCCACGCCCAGTCGCGTACCGTCTTCATGGGAGAAAAT
AATACTGTTGATGGGTGTCTGGTCAGAGACATCAAGAAATAACGCCGGAACATTAGTGCAGGCAGCTTCCACAGCAATGGCATCCTGGT
CATCCAGCGGATAGTTAATGATCAGCCCACTGACGCGTTGCGCGAGAAGATTGTGCACCGCCGCTTTACAGGCTTCGACGCCGCTTCGT
TCTACCATCGACACCACCACGCTGGCACCCAGTTGATCGGCGCGAGATTTAATCGCCGCGACAATTTGCGACGGCGCGTGCAGGGCCAG
ACTGGAGGTGGCAACGCCAATCAGCAACGACTGTTTGCCCGCCAGTTGTTGTGCCACGCGGTTGGGAATGTAATTCAGCTCCGCCATCG
CCGCTTCCACTTTTTCCCGCGTTTTCGCAGAAACGTGGCTGGCCTGGTTCACCACGCGGGAAACGGTCTGATAAGAGACACCGGCATAC
TCTGCGACATCGTATAACGTTACTGGTTTCACATTCACCACCCTGAATTGACTCTCTTCCGGGCGCTATCATGCCATACCGCGAAAGGT
TTTGCGCCATTCGATGGTGTCCGGGATCTCGACGCTCTCCCTTATGCGACTCCTGCATTAGGAAGCAGCCCAGTAGTAGGTTGAGGCCG
TTGAGCACCGCCGCCGCAAGGAATGGTGCATGCTCCCGGCCGCCATGGCGGCCGCGGGAATTCGATTTGGGCCTTTTGACAATAATCAT
CGGCTCGTATAATGTGTGGAATTGTGAGCGGATAACAATTGGGATCCGCTCTTTAACAATTTATCAGATCCAATAGGAGGAGAATCTAT
GGATAGCACTGAGAACGTCATCAAGCCCTTCATGCGCTTCAAGGTGCACATGGAGGGCTCCGTGAACGGCCACGAGTTCGAGATCGAGG
GCGTGGGCGAGGGCAAGCCCTACGAGGGCACCCAGACCGCCAAGCTGCAAGTGACCAAGGGCGGCCCCCTGCCCTTCGCCTGGGACATC
CTGTCCCCCCAGTTCTTCTACGGCTCCAAGGCGTACATCAAGCACCCCGCCGACATCCCCGACTACCTCAAGCAGTCCTTCCCCGAGGG
CTTCAAGTGGGAGCGCGTGATGAACTTCGAGGACGGCGGCGTGGTGACCGTGACCCAGGACTCCTCCCTGCAGGACGGCACCCTCATCT
ACCACGTGAAGTTCATCGGCGTGAACTTCCCCTCCGACGGCCCCGTAATGCAGAAGAAGACTCTGGGCTGGGAGCCCTCCACTGAGCGC
AACTACCCCCGCGACGGCGTGCTGAAGGGCGAGAACCACATGGCGCTGAAGCTGAAGGGCGGCGGCCACTACCTGTGTGAGTTCAAGTC
CATCTACATGGCCAAGAAGCCCGTGAAGCTGCCCGGCTACCACTACGTGGACTACAAGCTCGACATCACCTCCCACAACGAGGACTACA
CCGTGGTGGAGCAGTACGAGCGCGCCGAGGCCCACCACCACCTGTTCCAGTAGGTCGACAAGCTTGCGGCCGCACTCGAGCACCACCAC
CACCACCACCACCACTAATTGATTAATACCTAGGCTGCTAAACAAAGCCCGAAAGGAAGCTGAGTTGGCTGCTGCCACCGCTGAGCAAT
AACTAGCATAACCCCTTGGGGCCTCTAAACGGGTCTTGAGGGGTTTTTTGCTGAAAGGAGGAACTATATCCGGATTGGCGAATGGGACG
CGCCCTGTAGCGGCGCATTAAGCGCGGCGGGTGTGGTGGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATCGCCCTGATAGACGGTTTTTCGCCCTT
TGACGTTGGAGTCCACGTTCTTTAATAGTGGACTCTTGTTCCAAACTGGAACAACACTCAACCCTATCTCGGTCTATTCTTTTGATTTA
TAAGGGATTTTGCCGATTTCGGCCTATTGGTTAAAAAATGAGCTGATTTAACAAAAATTTAACGCGAATTTTAACAAAATATTAACGTT
TACAATTTCAGGTGGCACTTTTCGGGGAAATGTGCGCGGAACCCCTATTTGTTTATTTTTCTAAATACATTCAAATATGTATCCGCTCA
TGAATTAATTCTTAGAAAAACTCATCGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTATCGGTCTGCGATTCCGACTCGTCCAA
CATCAATACAACCTATTAATTTCCCCTCGTCAAAAATAAGGTTATCAAGTGAGAAATCACCATGAGTGACGACTGAATCCGGTGAGAAT
GGCAAAAGTTTATGCATTTCTTTCCAGACTTGTTCAACAGGCCAGCCATTACGCTCGTCATCAAAATCACTCGCATCAACCAAACCGTT
ATTCATTCGTGATTGCGCCTGAGCGAGACGAAATACGCGATCGCTGTTAAAAGGACAATTACAAACAGGAATCGAATGCAACCGGCGCA
GGAACACTGCCAGCGCATCAACAATATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAATTCCGTCAGCCAGTTTAGTCTGACCAT
CTCATCTGTAACATCATTGGCAACGCTACCTTTGCCATGTTTCAGAAACAACTCTGGCGCATCGGGCTTCCCATACAATCGATAGATTG
TCGCACCTGATTGCCCGACATTATCGCGAGCCCATTTATACCCATATAAATCAGCATCCATGTTGGAATTTAATCGCGGCCTAGAGCAA
GACGTTTCCCGTTGAATATGGCTCATAACACCCCTTGTATTACTGTTTATGTAAGCAGACAGTTTTATTGTTCATGACCAAAATCCCTT
AACGTGAGTTTTCGTTCCACTGAGCGTCAGACCCCGTAGAAA

TABLE 5
Sequences of codon optimized proteins

>codon optimised integrase from phage φC31 (SEQ ID NO: 20)
ATGCACCATCACCATCACCATGGTAGCGGCGACACGTACGCGGGTGCTTACGATCGCCAATCTCGTGAACGTGAAAACAGCTCAGCC
GCGTCCCCCGCAACCCAACGCTCCGCGAATGAGGATAAAGCCGCAGATCTGCAACGTGAGGTGGAACGTGATGGTGGTCGCTTTCGT
TTTGTGGGTCACTTTTCAGAGGCTCCAGGGACCAGCGCTTTTGGTACAGCCGAACGTCCTGAATTTGAGCGTATTTTAAATGAGTGT
CGTGCGGGCCGCTTAAATATGATTATCGTGTACGATGTTTCTCGCTTTTCTCGTCTCAAAGTGATGGATGCAATCCCCATCGTAAGC
GAGTTACTGGCGTTAGGTGTCACCATCGTCTCAACCCAAGAGGGTGTGTTTCGCCAAGGCAATGTCATGGACCTGATTCACCTGATC
ATGCGTCTGGATGCCTCACACAAGGAGAGCAGTTTAAAGAGCGCCAAAATCCTGGATACTAAGAATTTACAACGTGAGCTGGGTGGA
TATGTGGGCGGTAAAGCgccttacggcttcgagcttgTTAGCGAAACCAAAGAAATTACCCGTAATGGTCGCATGGTTAACGTGGTG
ATTAATAAACTGGCCCATTCAACAACCCCGCTGACAGGCCCGTTTGAATTTGAACCAGATGTCATTCGTTGGTGGTGGCGCGAAATT
AAAACTCATAAGCATCTGCCGTTTAAACCAGGTTCCCAGGCGGCGATCCATCCTGGTTCAATTACCGGCTTGTGCAAACGTATGGAT
GCGGATGCGGTTCCAACGCGTGGTGAAACCATCGGCAAGAAAACTGCGAGCTCGGCGTGGGACCCCGCCACAGTGATGCGCATTTTA
CGCGATCCCCGCATCGCCGGATTTGCTGCGGAAGTCATTTATAAGAAGAAACCAGATGGGACCCCAACGACCAAAATCGAAGGGTAT
CGTATCCAACGTGATCCTATTACCCTGCGTCCTGTTGAACTGGACTGTGGCCCCATTATTGAACCTGCGGAATGGTACGAACTGCAA
GCCTGGCTGGATGGTCGCGGTCGTGGTAAAGGTCTGAGCCGTGGTCAGGCGATCTTAAGCGCGATGGATAAACTTTATTGTGAATGC
GGTGCGGTTATGACATCCAAACGTGGCGAGGAGTCAATTAAAGATTCATATCGTTGTCGCCGTCGCAAAGTCGTTGATCCCTCGGCG
CCGGGCCAACATGAGGGAACTTGTAATGTGTCTATGGCCGCGCTGGATAAATTTGTAGCCGAGCGTATCTTTAATAAAATTCGTCAT
GCAGAGGGTGATGAGGAAACCCTGGCATTGTTATGGGAAGCGGCGCGCCGTTTTGGTAAACTTACCGAAGCCCCGGAGAAATCAGGA
GAGCGCGCCAATCTGGTGGCCGAACGTGCGGATGCGCTGAACGCCCTTGAAGAGCTGTATGAGGATCGTGCGGCCGGGGCATATGAT
GGTCCGGTCGGTCGTAAACATTTTCGCAAACAGCAAGCGGCATTAACCTTACGTCAACAGGGCGCCGAGGAACGTCTGGCAGAGCTC
GAGGCGGCCGAGGCACCCAAATTGCCGCTGGATCAGTGGTTTCCGGAAGATGCGGATGCGGACCCAACAGGTCCGAAAAGCTGGTGG
GGCCGTGCAAGTGTGGATGATAAACGTGTATTTGTGGGCCTGTTTGTTGATAAAATTGTGGTGACCAAATCAACGACCGGACGCGGC
CAAGGCACCCCGATTGAGAAACGTGCGAGTATTACTTGGGCTAAACCCCCAACGGATGATGATGAGGATGATGCTCAAGACGGCACG
GAAGACGTAGCGGCGTAA
>codon optimised integrase from phage Bxb1 (SEQ ID NO: 21)
ATGCGCGCGCTTGTTGTGATTCGTTTATCGCGTGTGACGGACGCCACCACCAGTCCCGAACGCCAATTAGAAAGCTGTCAACAACTT
TGTGCTCAACGTGGTTGGGATGTGGTGGGAGTGGCCGAAGACCTTGATGTTTCGGGCGCTGTTGACCCATTTGATCGCAAACGTCGT
CCTAATTTGGCGCGTTGGCTGGCCTTTGAAGAACAGCCTTTCGATGTAATTGTCGCATATCGCGTTGACCGTCTGACACGTTCCATT
CGCCACCTGCAACAACTTGTGCATTGGGCGGAAGATCATAAGAAATTAGTTGTTTCGGCAACAGAGGCCCATTTTGACACCACCACA
CCTTTCGCTGCTGTAGTTATTGCCCTGATGGGTACTGTCGCTCAAATGGAGCTCGAGGCAATTAAGGAACGCAATCGCTCCGCGGCC
CACTTTAACATTCGTGCTGGCAAGTATCGCGGCAGCTTACCCCCGTGGGGTTATCTCCCCACACGTGTAGATGGCGAATGGCGCCTC
GTCCCTGATCCGGTCCAACGCGAACGTATTCTGGAAGTCTACCATCGTGTAGTGGATAATCATGAACCCTTACACCTCGTTGCTCAT
GATCTCAATCGCCGCGGCGTTTTGAGCCCCAAAGATTATTTTGCCCAATTGCAGGGTCGTGAACCCCAAGGTCGCGAATGGTCCGCC
ACAGCCTTAAAACGCTCAATGATTTCTGAAGCAATGCTTGGCTATGCAACGTTGAATGGCAAAACTGTTCGTGATGATGATGGCGCG
CCCTTGGTTCGCGCCGAACCAATTTTGACACGCGAACAATTAGAAGCTCTTCGTGCAGAACTGGTTAAAACATCGCGTGCCAAACCG
GCCGTTAGTACTCCTTCTCTTCTCCTTCGTGTCCTGTTTTGTGCTGTCTGTGGCGAACCTGCCTATAAATTTGCTGGTGGCGGGCGC
AAACATCCTCGTTATCGTTGTCGTAGCATGGGCTTTCCCAAACATTGTGGCAATGGTACTGTTGCTATGGCGGAATGGGATGCCTTT
TGTGAAGAACAAGTGTTAGACCTTCTGGGCGATGCAGAACGCCTCGAAAAGGTATGGGTCGCCGGGAGCGATAGCGCAGTGGAGCTG
GCTGAAGTAAATGCCGAACTCGTTGATTTAACTAGCTTGATTGGATCTCCTGCGTATCGCGCCGGATCGCCACAACGTGAGGCCTTA
GACGCGCGCATCGCTGCCTTAGCTGCCCGCCAGGAAGAACTCGAAGGTTTGGAAGCGCGTCCTAGCGGATGGGAATGGCGTGAAACT
GGTCAACGCTTTGGAGATTGGTGGCGCGAACAAGATACGGCTGCCAAGAATACATGGCTGCGTTCCATGAATGTGCGTCTTACCTTT
GATGTGCGTGGTGGTTTAACCCGTACAATTGATTTTGGCGACCTGCAAGAATATGAACAACACCTGCGTCTGGGATCAGTCGTGGAG
CGCTTGCATACAGGCATGTCATAA
>codon optimised repressor cI857 (SEQ ID NO: 22)
ATGTCTACCAAAAAAAAACCACTTACACAGGAACAGTTGGAAGATGCCCGGCGTCTTAAGGCGATTTATGAGAAGAAGAAAAACGAA
TTAGGGCTGTCTCAAGAATCAGTGGCTGATAAGATGGGTATGGGACAGTCCGGCGTCGGGGCGTTATTTAATGGTATAAACGCATTA
AATGCCTACAACGCCGCTTTACTGACCAAAATTTTGAAAGTCTCCGTAGAAGAATTTAGTCCATCTATTGCTAGAGAGATATACGAG
ATGTACGAAGCCGTTAGTATGCAACCATCGCTGCGTTCGGAGTATGAATATCCAGTCTTTTCTCATGTCCAAGCCGGAATGTTTTCC
CCCGAGTTGCGGACATTTACGAAGGGAGACGCAGAGCGTTGGGTGTCGACAACTAAGAAAGCCTCCGACTCTGCCTTCTGGCTGGAG
GTAGAAGGAAACTCTATGACCGCCCCTACTGGATCTAAGCCTTCCTTCCCTGATGGGATGTTGATTCTGGTTGATCCTGAACAAGCG
GTTGAGCCCGGGGATTTCTGCATTGCAAGATTAGGCGGCGACGAATTTACTTTCAAAAAACTTATTCGCGATTCGGGACAAGTTTTC
CTGCAACCACTGAACCCGCAATACCCAATGATCCCTTGTAATGAATCATGCAGTGTTGTTGGTAAGGTGATAGCTTCGCAGTGGCCA
GAGGAGACCTTTGGGTAA

Results

DNA replication is a prerequisite for cell division in all organisms. In bacteria chromosomes carry a single DNA sequence of origin of replication (ori) whereon the replication complex assembles and replication begins. In E. coli the ori sequence is called oriC, which is situated between mnmG and mioC genes in common laboratory strains (Messer, 2002).

We attempted to block cell growth and division by inhibiting its DNA replication. In order to achieve that we integrated serine recombinase recognition sites attB and attP on either side of the oriC (FIG. 1A). When the corresponding serine recombinase is expressed in the cells, it catalyses cleavage of the DNA at the sites, excising the DNA sequence in between by strand exchange, and re-joining the DNA to form an essentially irreversible hybrid att site (Merrick, Zhao and Rosser, 2018). As a result, oriC is eliminated from the bacterial chromosome. Hereafter in this Examples section and elsewhere herein we call excision of oriC as “switch” or “switched” or “switcher” referring to a strain capable of switching. Excision of oriC will exclude any further DNA replication initiation while allowing the ongoing replication to finish. In these Examples, we are using serine recombinases to build the switch, but it is clear that other types of genome editing can be used for the same purpose. Possible candidates include, but are not limited to, tyrosine recombinases (Fogg et al., 2014), CRISPR/Cas (Yao et al., 2018) or any other type of enzyme that permanently alters the oriC sequence in chromosome and leaves it incapable to initiate DNA replication.

Essentially, the serine recombinase (or other type of editing) activity/expression is tightly controlled to avoid premature elimination of oriC and cessation of growth. We used a temperature-sensitive phage lambda c1857 repressor for that purpose (FIG. 1B). At 30° C. (or lower) it is bound to its target promoter (P_{R} or PO and represses it, while at 37° C. (or higher) the repression is relieved, and expression is turned on. Other types of gene expression control methods can be used for the same purpose, including but not limited to, induction by inducers (IPTG, arabinose, homoserine lactones, anhydrotetracycline etc) (Marschall, Sagmeister and Herwig, 2017)(Lutz and Bujard, 1997)(Cox, Surette and Elowitz, 2007), targeted proteolysis (Cameron and Collins, 2014), different variants of CRISPR/Cas9 (Qi et al., 2013) etc.

We constructed an E. coli strain that carries a modified oriC vicinity (FIG. 1A) and a serine recombinase expression system, expressed on an episomal plasmid (although equally the serine recombinase expression system could be integrated into the genome—as assessed in FIGS. 7B and 7C). In our system we can express different serine recombinases in a single or dual settings under the control of the same cl repressor (FIGS. 1B and 1C). Additional recombinases can be added, with same or different expression control mechanisms, if necessary. We used φC31 integrase in our experiments, but it is clear that other serine recombinases, together with their cognate attP and attB sites, can be used for the same purpose. There are tens of different serine recombinases described experimentally and more than 4000 new ones predicted by bioinformatics (Yang et al., 2014).

It is possible that rare mutants arise that have lost their ability to stop growth in response to temperature shift. Such mutants (“cheaters”) will continue to grow and multiply at 37° C., and could become a dominant population in culture. To avoid this potential problem it is possible to include positive selection only for cells that have performed the origin of replication (oriC) elimination from the genome. If the latter switches on the expression of the gene necessary for survival (for example antibiotic resistance gene or a gene necessary for metabolism of an essential nutrient or for metabolism of an artificial nitrogen source), only the cells that have switched survive.

We tested our switcher's ability to perform the elimination of oriC from the genome in a time course experiment by at first growing the cells at 30° C. (no excision) and then transferring the culture to 37° C. (excision) (FIG. 2). After temperature shift, CFU/mL numbers drop in switcher strain by more than two orders of magnitude (FIG. 2A) and recombination quickly eliminates oriC (FIG. 2B). None of these changes is happening in a control strain that lacks active recombinase (Control). To further confirm excision of oriC, we sequenced the junction between the attB and attP sites in a switched strain and verified it to be exactly as predicted (FIG. 2C).

It was found that excision of oriC and elimination of genome replication eventually led to a stop in cell division and growth. In order to investigate the changes of cell density in a batch culture system, we induced the switch at different cell densities (FIG. 3). After the switch, the OD values continue to increase for few hours and then start slowing down. Depending on the initial cell density, the switcher strain gradually reaches a distinct density plateau (approximately 65%, 95%, and 110% of control strain plateau in FIG. 3) that can be even higher than in control cells. Switcher cultures at final densities lower than controls suggest that nutrients are not exhausted from the medium and growth of the switcher is stopped by other means. This allows us to tune the level of the plateau by just choosing the appropriate OD (cell density) at the time of the switch.

To assess protein production capability of the switcher strain we used a plasmid-based fluorescent reporter system that encodes for monomeric red fluorescent protein (mRFP1) under the control of homoserine lactone (HSL) inducible promoter. Addition of HSL to the culture medium results in mRFP production that can be monitored by measuring red fluorescence at (ex: 584 nm, em: 607 nm). Both the switcher and the control were transformed with the reporter plasmid and tested in a temperature shift experiment (FIG. 4).

After growing these strains for four hours at 30° C. the cultures were shifted to 37° C. and HSL was added at the same time to induce mRFP1 synthesis (FIG. 4A). Both cultures start to synthesize mRFP1, but the synthesis stops in control culture as it reaches stationary phase. Although the cell density stops growing also in switcher culture, the mRFP1 level continues to increase and reaches higher level compared to control. Even higher mRFP1 expression is reached when the switch is induced at higher density and the switcher culture reaches the same OD as control culture (FIG. 4B). The synthesis of mRFP1 is much more efficient in switcher strain compared to the control strain as mRFP1 fluorescence over OD ratio almost triples (FIGS. 4C and 4D). This demonstrates a significant advantage of our switcher system over the control in protein expression.

To test the protein synthesis capability of switched cells in longer period of time, we varied HSL addition time upon the switch (FIG. 5). The switcher strain was able to induce mRFP1 synthesis after 3, 6, 20 or even 30 hours after the switch while in the control strain the mRFP1 induction is detectable only after 3 hours (FIG. 5B). There is some increase of mRFP1 signal over time in HSL-negative switcher culture, but the addition of HSL clearly induces mRFP1 expression. This result shows, that despite their non-growing status the switcher cells remain in metabolically active state and are able to respond to external stimulus and initiate new protein synthesis long after the switch has occurred.

To confirm that the positive effect the switcher has on protein expression is not specific to a particular reporter plasmid we tested switching also with a different reporter. A fluorescent reporter system that encodes for crimson fluorescent protein (E2-Crimson) under the control of isopropyl β-D-1-thiogalactopyranoside (IPTG) inducible Ptac promoter was combined with p15A origin of replication and ampicillin resistance gene for selection (reporter plasmid pAJ157). Addition of IPTG to the culture medium induces E2-Crimson production which can be monitored by measuring fluorescence (ex: 610 nm, em: 650 nm). Both the switcher and the control were transformed with the reporter plasmid and tested in a temperature shift experiment (FIG. 7A). After growing these strains for four hours at 30° C. the cultures were shifted to 37° C. and IPTG was added at the same time to induce E2-Crimson synthesis. Both cultures start to synthesize E2-Crimson, but the switcher strain produces significantly more fluorescent protein.

To construct a plasmid-free switcher strain, all genetic elements necessary for the switcher to function were combined into the genome of E. coli MG1655 resulting in bAJ174 (genomic switcher; Table 3, FIG. 8A). Integrase from phage φC31 was codon optimised for enhanced synthesis in E. coli and kept under the control of λP_{R}(T41C) promoter. To enhance genomic repression of integrases at 30° C., in addition to wild-type c1857 an extra copy of codon-optimised c1857 under the control of PkatG promoter was added to the genome. Additionally, integrase from phage Bxb1 was codon-optimised and placed under the control of λP_{L} promoter and respective attB and attP sites were added to either side of oriC to increase the probability of successful recombination.

Genomic switcher efficiently stopped the cell growth and increased the synthesis of mRFP1 and E2-Crimson proteins (FIG. 7B, C). This demonstrates that all the essential elements can be incorporated into bacterial genome, thus switcher cells can function free of plasmids.

### REFERENCES

Brockman, I. M. and Prather, K. L. (2015) ‘Dynamic knockdown of E. coli central metabolism for redirecting fluxes of primary metabolites. TL-28’, Metabolic engineering, 28 VN-r, pp. 104-113. doi: 10.1016/j.ymben.2014.12.005.
Burg, J. M. et al. (2016) large-scale bioprocess competitiveness: the potential of dynamic metabolic control in two-stage fermentations TL-14′, Current Opinion in Chemical Engineering. sciencedirect, 14 VN-r, pp. 121-136. doi: 10.1016/j.coche.2016.09.008.
Cameron, D. E. and Collins, J. J. (2014) ‘Tunable protein degradation in bacteria’, Nature Biotechnology. Nature Publishing Group, 32(12), pp. 1276-1281. doi: 10.1038/nbt.3053.
Campbell, R. E. et al. (2002) ‘A monomeric red fluorescent protein’, Proceedings of the National Academy of Sciences of the United States of America. National Academy of Sciences, 99(12), pp. 7877-7882. doi: 10.1073/pnas.082243699.
Cox, R. S., Surette, M. G. and Elowitz, M. B. (2007) ‘Programming gene expression with combinatorial promoters’, Molecular Systems Biology. John Wiley & Sons, Ltd, 3(1), p. 145. doi: 10.1038/msb4100187.
Datsenko, K. A. and Wanner, B. L. (2000) One-step inactivation of chromosomal genes in Escherichia coli K-12 using PCR products. Available at: www.pnas.orgcgidoi10.1073pnas.120163297 (Accessed: 12 Apr. 2019).
Van Dien, S. (2013) ‘From the first drop to the first truckload: commercialization of microbial processes for renewable chemicals.’, Curr. Opin. Biotechnol., 24(6), pp. 1061-1068. doi: 10.1016/j.copbio.2013.03.002.
Fogg, P. C. M. et al. (2014) ‘New Applications for Phage Integrases TL-426’, Journal of Molecular Biology. sciencedirect, 426 VN-(15), pp. 2703-2716. doi: 10.1016/j.jmb.2014.05.014.
Jechlinger, W. et al. (1999) ‘Altered temperature induction sensitivity of the lambda p R/c1857 system for controlled gene E expression in Escherichia coli’, FEMS Microbiology Letters.
Wiley/Blackwell (10.1111), 173(2), pp. 347-352. doi: 10.1111/j.1574-6968.1999.tb13524.x.
Lee, J. W. et al. (2018) ‘Next-generation biocontainment systems for engineered organisms’, Nature Chemical Biology. Nature Publishing Group, 14(6), pp. 530-537. doi: 10.1038/s41589-018-0056-x.
Lutz, R. and Bujard, H. (1997) ‘Independent and Tight Regulation of Transcriptional Units in Escherichia Coli Via the LacR/O, the TetR/O and AraC/11-12 Regulatory Elements I Nucleic Acids Research I Oxford Academic’, Nucleic Acids Research, 25(6), pp. 1203-1210. Available at: https://academic.oup.com/nar/article/25/6/1203/1197243 (Accessed: 19 Jun. 2020).
Ma, Y. et al. (2018) ‘Identification and application of a growth-regulated promoter for improving 1-valine production in Corynebacterium glutamicum’, Microbial Cell Factories. BioMed Central Ltd., 17(1), p. 185. doi: 10.1186/s12934-018-1031-7.
Madsen, S. M. et al. (1999) ‘Molecular characterization of the pH-inducible and growth phase-dependent promoter P170 of Lactococcus lactis’, Molecular Microbiology. Blackwell Publishing Ltd., 32(1), pp. 75-87. doi: 10.1046/j.1365-2958.1999.01326.x.
Marschall, L., Sagmeister, P. and Herwig, C. (2017) ‘Tunable recombinant protein expression in E. coli: promoter systems and genetic constraints’, Applied Microbiology and Biotechnology. Springer Verlag, 101(2), pp. 501-512. doi: 10.1007/s00253-016-8045-z.
Merrick, C., Zhao, J. and Rosser, S. (2018) ‘Serine Integrases: Advancing Synthetic Biology’, ACS Synthetic Biology. acs. doi: 10.1021/acssynbio.7b00308.
Messer, W. (2002) The bacterial replication initiator DnaA. DnaA and oriC, the bacterial mode to initiate DNA replication TL-26′, FEMS Microbiology Reviews. wiley, 26 VN-r(4), pp. 355-374. doi: 10.1111/j.1574-6976.2002.tb00620.x.
Paul, S. et al. (2004) ‘Autoinduction of Bacillus subtilis phoPR operon transcription results from enhanced transcription from EaA- and EaE-responsive promoters by phosphorylated PhoP’, Journal of Bacteriology. American Society for Microbiology Journals, 186(13), pp. 4262-4275. doi: 10.1128/JB.186.13.4262-4275.2004.
Qi, L. S. et al. (2013) ‘Repurposing CRISPR as an RNA-guided platform for sequence-specific control of gene expression’, Cell. Cell Press, 152(5), pp. 1173-1183. doi: 10.1016/j.ce11.2013.02.022.
Soma, Y. et al. (2014) ‘Metabolic flux redirection from a central metabolic pathway toward a synthetic pathway using a metabolic toggle switch.’, Metab. Eng. doi: 10.1016/j.ymben.2014.02.008.
Venayak, N. et al. (2015) ‘Engineering metabolism through dynamic control’, Curr Opin Biotech, 34, pp. 142-152. doi: 10.1016/j.copbio.2014.12.022.
Wang, J. et al. (2018) ‘A Novel Process for Cadaverine Bio-Production Using a Consortium of Two Engineered Escherichia coli’, Frontiers in Microbiology. Frontiers Media S.A., 9(JUN), p. 1312. doi: 10.3389/fmicb.2018.01312.
Yang, L. et al. (2014) ‘Permanent genetic memory with >1-byte capacity’, Nature Methods. Nature Publishing Group, 11(12), pp. 1261-1266. doi: 10.1038/nmeth.3147.
Yao, R. et al. (2018) ‘CRISPR-Cas9/Cas12a biotechnology and application in bacteria’, Synthetic and Systems Biotechnology. KeAi Communications Co., pp. 135-149. doi: 10.1016/j.synbio.2018.09.004.
Yuan, S. F. et al. (2020) ‘De novo resveratrol production through modular engineering of an Escherichia coli-Saccharomyces cerevisiae co-culture’, Microbial Cell Factories. BioMed Central, 19(1), p. 143. doi: 10.1186/s12934-020-01401-5. Strack, R. L. et al. (2009) ‘A rapidly maturing far-red derivative of DsRed-Express2 for whole-cell labeling.’, Biochemistry, 48(35), pp. 8279-8281. doi: 10.1021/bi900870u.

1. A method of producing a product by a bacterial cell, the method comprising a step of irreversibly inactivating an origin of replication in the bacterial cell.

2. The method of claim 1, wherein the method increases the yield or titer of the product produced by the bacterial cell, or increases the productivity of the bacterial cell.

3. The method of claim 1 or claim 2, wherein the product is a heterologous product.

4. The method of claim 1 or claim 2, wherein the product is a homologous or endogenous product.

5. The method of any one of claims 1 to 4, wherein the step of irreversibly inactivating an origin of replication in the bacterial cell comprises genetic modification of the origin of replication.

6. The method of claim 5, wherein the genetic modification comprises at least partial removal of the nucleotide sequence of the origin of replication.

7. The method of claim 6, wherein the genetic modification comprises removal of the full nucleotide sequence of the origin of replication.

8. The method of any of claims 1 to 7, said method comprising:
(i) providing a bacterial cell producing or capable of producing said product;
(ii) irreversibly inactivating an origin of replication in the bacterial cell; and optionally
(iii) inducing production of the product in the bacterial cell.

9. The method of claim 8, wherein steps (ii) and (iii) take place simultaneously or sequentially.

10. The method of claim 8 or claim 9, wherein the bacterial cell exists within a bacterial cell population, and wherein steps (ii) and/or (iii) take place during the exponential growth phase of the bacterial cell population.

11. A method of stopping growth of a bacterial cell without impairing the metabolic activity of the bacterial cell, the method comprising irreversibly inactivating an origin of replication in the bacterial cell.

12. A bacterial cell comprising an origin of replication, wherein the bacterial cell has been modified such that the origin of replication can be irreversibly inactivated.

13. The bacterial cell of claim 12, wherein the origin of replication is flanked by site-specific recombination sites for recognition by site-specific recombinase.

14. The bacterial cell of claim 13, wherein the site-specific recombination sites comprise site-specific recombination sites for recognition by a serine recombinase, preferably wherein the serine recombinase is γδ, Bxb1, φC31 or TP901, and/or wherein the site-specific recombination sites comprise site-specific recombination sites for recognition by a tyrosine recombinase, preferably wherein the tyrosine recombinase is Cre, Dre, Flp, KD, B2 or B3.

15. The bacterial cell of claim 13 or claim 14, wherein the site-specific recombination sites comprise a pair of site-specific recombination sites, and/or wherein the site-specific recombination sites comprise two or more pairs of site-specific recombination sites.

16. The bacterial cell of any one of claims 13 to 15, further comprising a gene encoding the site-specific recombinase, and/or a gene encoding a heterologous product.

17. The bacterial cell of claim 16, wherein the gene encoding the site-specific recombinase is operatively linked to a promoter, preferably wherein the promoter is temperature-sensitive, pH-sensitive, light-sensitive, or chemically-sensitive; more preferably wherein the promoter is regulated by phage lambda c1857 repressor.

18. A modified bacterial cell, wherein said modified bacterial cell lacks a functional origin of replication sequence.

19. The modified bacterial cell of claim 18, wherein said cell is obtainable by a method comprising a step of irreversibly inactivating an origin of replication in the bacterial cell.

20. The method of any one of claims 1 to 11, wherein said bacterial cell is as defined in any one of claims 12 to 19.

21. A polynucleotide vector comprising an origin of replication flanked by site-specific recombination sites.

22. The polynucleotide vector of claim 21, wherein the site-specific recombination sites comprise site-specific recombination sites for recognition by a serine recombinase, preferably wherein the serine recombinase is γδ, Bxb1, φC31 or TP901; and/or wherein the site-specific recombination sites comprise site-specific recombination sites for recognition by a tyrosine recombinase, preferably wherein the tyrosine site-specific recombinase is Cre, Dre, Flp, KD, B2 or B3.

23. Use of the bacterial cell of any one of claims 12 to 17, the modified bacterial cell of claim 18 or claim 19, or the polynucleotide vector of claim 21 or claim 22, for producing a product.

24. The bacterial cell of any one of claims 12 to 17, or the modified bacterial cell of claim 18 or claim 19, for use in the treatment or prevention of a disease or pathology, preferably wherein the disease or pathology is cancer, metabolic disease or an immunological disorder.

25. The method, bacterial cell, modified bacterial cell, polynucleotide vector, use, or bacterial cell for use, of any one of claims 1 to 24, wherein the bacterial cell is Escherichia sp., Bacillus sp., Lactococcus sp., Streptococcus sp., Lactobacillus sp., Corynebacterium sp., Streptomyces sp., Pseudomonas sp., Clostridium sp., Xanthomonas sp, or Enterobacteriaceae.
